(19) United States
(12) Patent Application Publication
(10) Pub. No.: US 2024/0158368 A1
(43) Pub. Date: May 16, 2024

(54) Pyrrolidine Main Protease Inhibitors as Antiviral Agents

(71) Applicant: AbbVie Inc., North Chicago, IL (US)

(72) Inventors: David A. Degoey, Salem, WI (US); Michael R. Schrimpf, Grayslake, IL (US); David J. Hardee, Vernon Hills, IL (US); Jacob Ludwig, Waukegan, IL (US); Eric R. Miller, Palatine, IL (US); Timothy R. Hodges, Milwaukee, WI (US); Alberto Munoz, North Chicago, IL (US); Sarah J. Perlmutter, Grayslake, IL (US); Huan-Qiu X. Li, Wilmette, IL (US); Alvin Jang, North Chicago, IL (US); Elizabeth L. Noey, St. Francis, WI (US); Gregory A. Gfesser, Lindenhurst, IL (US); Edgars Jecs, North Chicago, IL (US); Robert G. Schmidt, Antioch, IL (US); Justin D. Dietrich, Lake Villa, IL (US); Xenia B. Searle, Bodega Bay, CA (US); Boguslaw P. Nocek, Indianapolis, IN (US); Andrew Bogdan, Evanston, IL (US)

(73) Assignee: AbbVie Inc., North Chicago, IL (US)

(21) Appl. No.: 18/379,351

(22) Filed: Oct. 12, 2023

Related U.S. Application Data
(60) Provisional application No. 63/416,037, filed on Oct. 14, 2022.
(60) Provisional application No. 63/489,389, filed on Mar. 9, 2023.

(51) Int. Cl. C07D 401/12; A61P 31/14

(52) U.S. Cl. CPC C07D 401/12; A61P 31/14

(57) ABSTRACT

Pyrrolidine main protease inhibitors are described that are effective as antiviral compounds.

### CROSS-REFERENCE TO RELATED APPLICATIONS

This application claims the benefit of U.S. Provisional Application 63/416,037, filed Oct. 14, 2022, and U.S. Provisional Application 63/489,389, filed Mar. 9, 2023, the disclosures of which are incorporated by reference herein in their entirety.

### FIELD OF THE INVENTION

This invention pertains to compounds which are effective as antiviral compounds through their inhibition of main protease (MPro) protein found in many viruses, including coronaviruses.

### BACKGROUND OF THE INVENTION

Coronavirus Disease 2019 (COVID-19) is caused by severe acute respiratory syndrome coronavirus 2 (SARS-CoV-2). Small molecule compounds that can reduce symptoms associated with infections of COVID-19 and other coronaviruses are urgently needed.

### SUMMARY OF THE INVENTION

In an embodiment, compounds were found that are effective as antiviral compounds having the Formula (III)

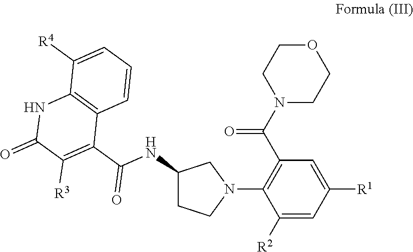

where: R¹ is CN or Br; R² is CF₃ or cyclopropyl; R³ is H or F; and R⁴ is H or F. Specific examples of compounds having the Formula (III) include Compound (1) and Compound (2) or pharmaceutically acceptable salts thereof.

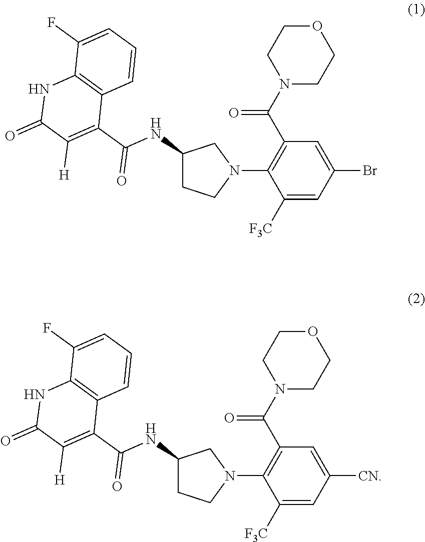 [SMILES: O=C(N[C@@H]1CCN(c2c(C(=O)N3CCOCC3)cc(Br)cc2C(F)(F)F)C1)c1cc(=O)[nH]c2c(F)cccc12 ; N#Cc1cc(C(=O)N2CCOCC2)c(N2CC[C@@H](NC(=O)c3cc(=O)[nH]c4c(F)cccc34)C2)c(C(F)(F)F)c1]

In an embodiment, compounds were found that are effective as antiviral compounds having the formula (VI)

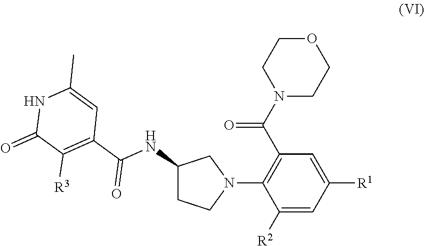

where: R¹ is CN or Br; R² is CF₃ or cyclopropyl; and R³ is H or F.

A Specific Example of a Compound Having the Formula (VI) is Compound (3) or a Pharmaceutically Acceptable Salt Thereof.

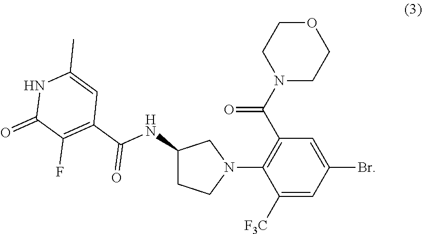 [SMILES: Cc1cc(C(=O)N[C@@H]2CCN(c3c(C(=O)N4CCOCC4)cc(Br)cc3C(F)(F)F)C2)c(F)c(=O)[nH]1]

In another embodiment, compounds were found that are effective antiviral compounds having the structure of Compound (1), Compound (2), Compound (3) or pharmaceutically acceptable salts thereof

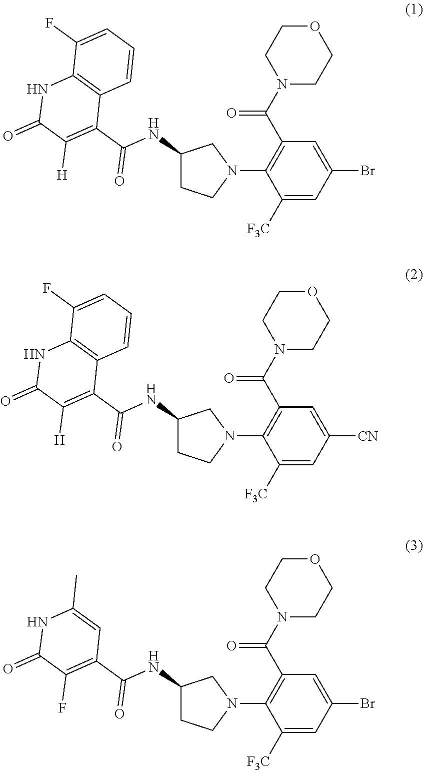 [SMILES: O=C(N[C@@H]1CCN(c2c(C(=O)N3CCOCC3)cc(Br)cc2C(F)(F)F)C1)c1cc(=O)[nH]c2c(F)cccc12 ; N#Cc1cc(C(=O)N2CCOCC2)c(N2CC[C@@H](NC(=O)c3cc(=O)[nH]c4c(F)cccc34)C2)c(C(F)(F)F)c1 ; Cc1cc(C(=O)N[C@@H]2CCN(c3c(C(=O)N4CCOCC4)cc(Br)cc3C(F)(F)F)C2)c(F)c(=O)[nH]1]

In another embodiment, the invention provides for a compound represented by the following structure

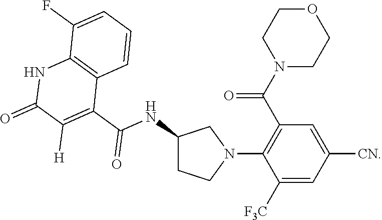 [SMILES: N#Cc1cc(C(=O)N2CCOCC2)c(N2CC[C@@H](NC(=O)c3cc(=O)[nH]c4c(F)cccc34)C2)c(C(F)(F)F)c1]

### DETAILED DESCRIPTION OF THE INVENTION

Coronaviruses are a large family of viruses that usually cause mild to moderate upper-respiratory tract illnesses in humans. However, three coronaviruses have caused more serious and fatal disease in people: SARS coronavirus (SARS-CoV) causes severe acute respiratory syndrome (SARS); MERS coronavirus (MERS-CoV) causes Middle East respiratory syndrome (MERS) and SARS-CoV-2 causes coronavirus disease 2019 (COVID-19). Other coronaviruses which have been identified and attributed to moderate respiratory infections include OC43 coronavirus, HKU1 coronavirus, 229E coronavirus, and NL63 coronavirus. Vaccines have proven effective at slowing the spread of COVID-19 and ameliorating the more severe symptoms associated with COVID-19 infections. The rise of variants of SARS-CoV-2, and the inherent lag time needed to develop new vaccines that are effective against these variants, create a need for effective small molecule compounds that can reduce the more severe side effects of infections of COVID-19 and other coronaviruses.

A common feature of these, and other viruses, is the presence of proteases that catalyze the cleavage of polyproteins associated with the virus. Cleavage of these polypeptides produces mature functional viruses. The action of these proteases is therefore essential for viral replication. Viral proteases make an attractive target for the development of broad-spectrum antiviral compounds. A potential protease target for broad-spectrum antiviral compounds is the main protease (MPro) protein found in many viruses, including coronaviruses.

Compounds were found that are effective as antiviral compounds through their inhibition of MPro. In one embodiment, an antiviral compound has the structure of Formula (I)

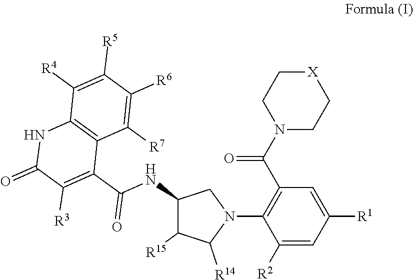

where:
R¹ is CN, Cl, Br, F, or C₁-C₆ alkyl;
R² is CF₃, CHF₂, OCF₃, OCHF₂, Cl, or cyclopropyl;
R³ is H or F;
R⁴ is H, F, or Cl;
R⁵ is H or F;
R⁶ is H or F;
R⁷ is H, OCH₃, or F;
X is O, CR¹⁰R¹¹, NR¹²;
R¹⁰, R¹¹, and R¹² are each independently H, C₁-C₆ alkyl, SO₂—(C₁-C₆ alkyl), or CN;
where R¹⁴ is H, C₁-C₆ alkyl, or ═O; and
where R¹⁵ is H, C₁-C₆ alkyl, or —OC₁-C₆ alkyl.

In some embodiments, at least one of R³, R⁴, R⁵, R⁶, or R⁷ is not H.

The term “alkyl” as used herein generally refers to a chemical substituent containing the monovalent group CₙH₂ₙ₊₁, where n is an integer greater than zero. The term “alkyl” includes a branched or unbranched monovalent unsubstituted hydrocarbon radical. In some embodiments, n is 1 to 6. Exemplary alky groups include, but are not limited to, methyl, ethyl, n-propyl, propyl, 2-propenyl (or allyl), n-butyl, t-butyl, and i-butyl (or 2-methylpropyl).

In one embodiment, an antiviral compound has the structure of Formula (II)

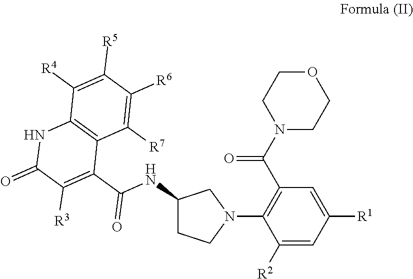

where:
R¹ is CN, Cl, Br, F, or C₁-C₆ alkyl;
R² is CF₃, CHF₂, OCF₃, OCHF₂, Cl, or cyclopropyl;
R³ is H or F;
R⁴ is H, F, or Cl;
R⁵ is H or F;
R⁶ is H or F; and
R⁷ is H, OCH₃, or F.

In some embodiments, at least one of R³, R⁴, R⁵, R⁶, or R⁷ is not H.

In one embodiment, an antiviral compound has the structure of Formula (III)

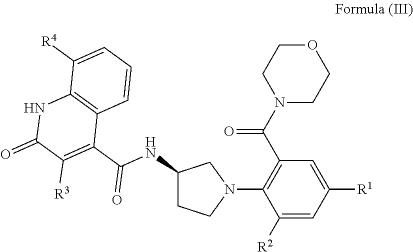

where:
R¹ is CN or Br;
R² is CF₃ or cyclopropyl;
R³ is H or F; and
R⁴ is H or F.

Specific examples of compounds that have Formula (III) include, but are not limited to compound (1) and (2)

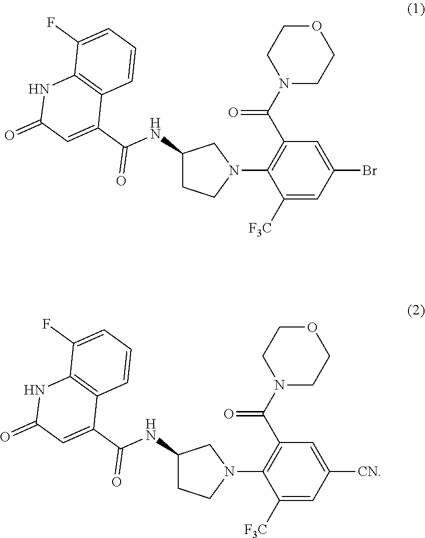 [SMILES: O=C(N[C@@H]1CCN(c2c(C(=O)N3CCOCC3)cc(Br)cc2C(F)(F)F)C1)c1cc(=O)[nH]c2c(F)cccc12 ; N#Cc1cc(C(=O)N2CCOCC2)c(N2CC[C@@H](NC(=O)c3cc(=O)[nH]c4c(F)cccc34)C2)c(C(F)(F)F)c1]

In one embodiment, an antiviral compound has the structure of Formula (IV)

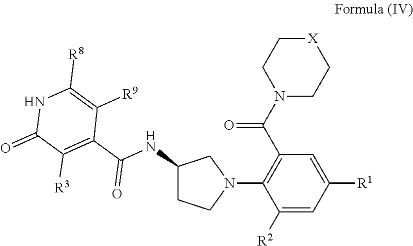

where:
R¹ is CN, Cl, Br, F, or C₁-C₆ alkyl;
R² is CF₃, CHF₂, OCF₃, OCHF₂, Cl, or cyclopropyl;
R³ is H or F;
R⁸ is H, CHF₂, C₁-C₆ alkyl, or OCH₃;
R⁹ is H, F, or Cl;
X is O, CR¹⁰R¹¹, or NR¹²; and
R¹⁰, R¹¹, and R¹² are each independently H, C₁-C₆ alkyl, SO₂—(C₁-C₆ alkyl), or CN.

In some embodiments, at least one of R³, R⁸, or R⁹ is not H.

In one embodiment, an antiviral compound has the structure of Formula (V)

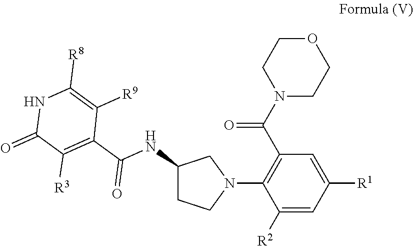

where:
R¹ is CN, Cl, Br, F, or C₁-C₆ alkyl;
R² is CF₃, CHF₂, OCF₃, OCHF₂, Cl, or cyclopropyl;
R³ is H or F;
R⁸ is H, CHF₂, C₁-C₆ alkyl, or OCH₃; and
R⁹ is H, F, or Cl.

In some embodiments, at least one of R³, R⁸, or R⁹ is not H.

In an embodiment, an antiviral compound has the structure of Formula (VI)

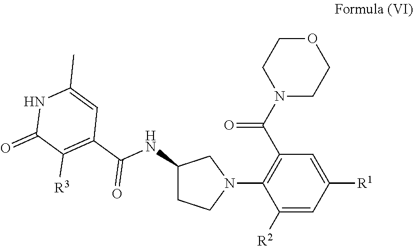

where:
R¹ is CN or Br;
R² is CF₃ or cyclopropyl; and
R³ is H or F.

A specific example of a compound that has Formula (VI) is Compound (3)

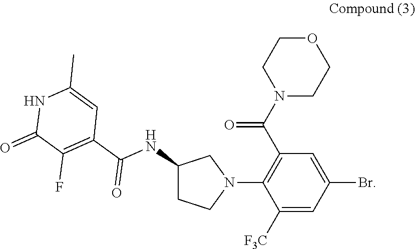 [SMILES: Cc1cc(C(=O)N[C@@H]2CCN(c3c(C(=O)N4CCOCC4)cc(Br)cc3C(F)(F)F)C2)c(F)c(=O)[nH]1]

In one embodiment, an antiviral compound has the structure of Formula (VII)

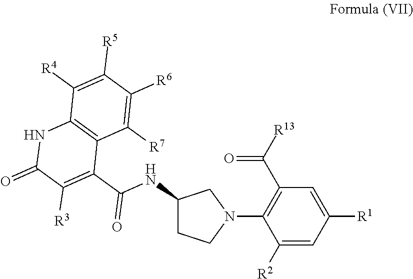

where:
R¹ is CN, Cl, Br, F, or C₁-C₆ alkyl;
R² is CF₃, CHF₂, OCF₃, OCHF₂, Cl, or cyclopropyl;
R³ is H or F;
R⁴ is H, F, or Cl;
R⁵ is H or F;
R⁶ is H or F;
R⁷ is H, OCH₃, or F; and
R¹³ is:

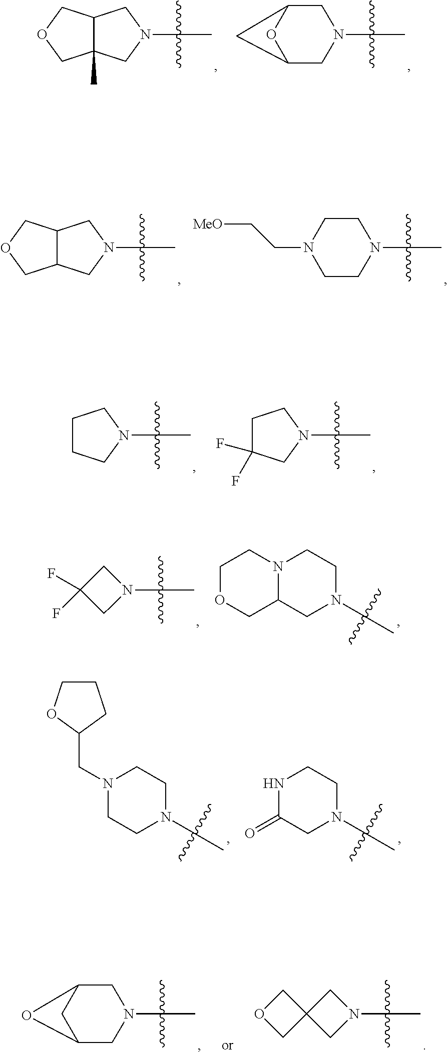

In some embodiments, at least one of R³, R⁴, R⁵, R⁶, or R⁷ is not H.

One or more compounds of this invention can be administered to a human patient by themselves or in pharmaceutical compositions where they are mixed with biologically suitable carriers or excipient(s) at doses to treat or ameliorate symptoms associated with infections of COVID-19 and other coronaviruses. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein.

The compounds their salts thereof described herein can be used to treat a subject suffering from a coronavirus infection. It is believed that the compounds reduce the ability of the coronavirus to proliferate in the subject by inhibiting the action of Mpro. Inhibition of Mpro disrupts the cleavage of polyproteins associated with the virus, preventing the virus from becoming a mature functional virus and/or from replicating. In an embodiment, a method of treating a subject suffering from a coronavirus infection (e.g., a COVID-19 infection) comprises administering a therapeutically effective amount of a compound described herein. Compounds that can be used to treat a coronavirus infection include but are not limited to compounds having Formula (III) and compounds having Formula (VI) as described herein. Specific compounds that can be used to treat a coronavirus infection include, but are not limited to, Compound (1), Compound (2), and Compound (3) as described herein.

### Synthesis of Antiviral Compounds

### Schemes

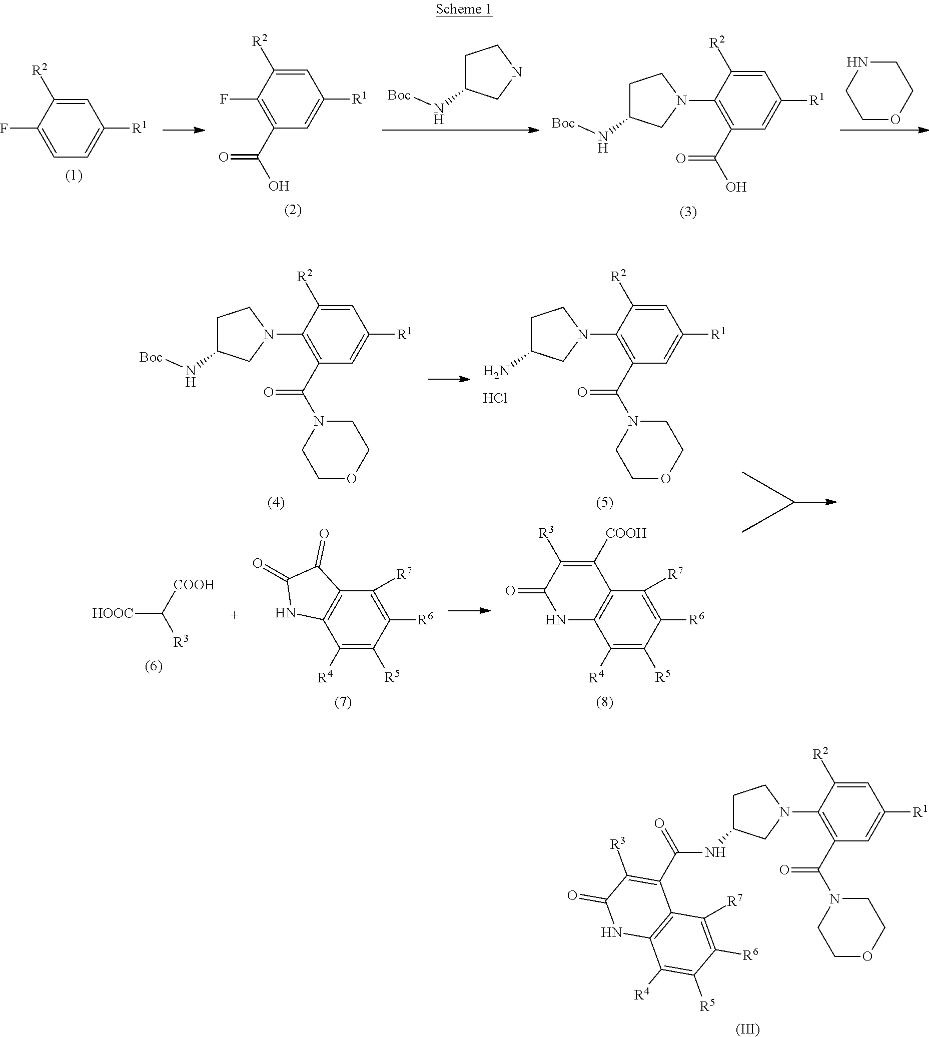

As shown in Scheme 1, compounds of formula (I), wherein R¹ and R² are as defined herein, can be treated with 2,2,6,6-tetramethylpiperidinylmagnesium chloride lithium chloride, followed by carbon dioxide gas, to provide compounds of formula (2). The reaction is typically performed at low temperature in a solvent such as, but not limited to, tetrahydrofuran. Reaction of compounds of formula (2) with (R)-tert-butyl pyrrolidin-3-ylcarbamate in the presence of a base such as, but not limited to, diisopropylethylamine, diisopropylamine or triethylamine can provide compounds of formula (3). The reaction is typically performed at an elevated temperature, and in a solvent such as, but not limited to, acetonitrile, N,N-dimethylformamide or N-methylpyrrolidinone. Compounds of formula (4) can be prepared by reacting compounds of formula (3) with morpholine in the presence of HATU (1-[bis(dimethylamino)methylene]-1H-1,2,3-triazolo[4,5-b]pyridinium 3-oxide hexafluorophosphate) followed by a base such as, but not limited to triethylamine. The reaction is typically performed while keeping the reaction mixture near ambient temperature in a solvent such as, but not limited to N,N-dimethylformamide. Removal of the Boc-group of compounds of formula (4) to provide compounds of formula (5) can be accomplished using an acid such as, but not limited to, hydrochloric acid in a solvent such as, but not limited to, cyclopentyl methyl ether.

Compounds of formula (6), wherein R³ is as defined herein, can be reacted with compounds of formula (7), wherein R⁴ is as defined herein, in acetic acid, to provide compounds of formula (8). The reaction is typically performed at an elevated temperature and may include a base such as sodium acetate.

Compounds of formula (8) can be treated with DMTMM (4-(4,6-dimethoxy-1,3,5-triazin-2-yl)-4-methylmorpholinium chloride), followed by addition to a mixture of compounds of formula (5), and N-methylmorpholine, in a solvent such as, but not limited to, N,N-dimethylformamide to provide compounds of Formula (III). The reaction is typically performed at ambient temperature.

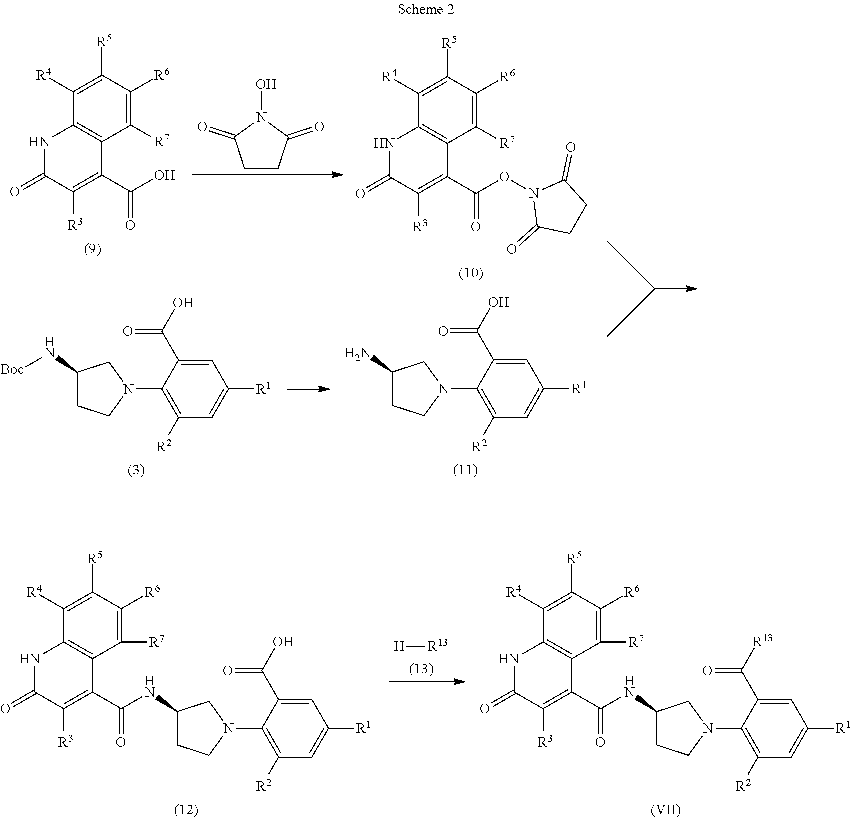

Scheme 2 shows the synthesis of compounds of Formula (VII). A quinolone acid compound of formula (9), wherein R³, R⁴, R⁵, R⁶, and R⁷ are as defined herein, can be converted to an activated ester species of formula (10) by treatment with N-hydroxysuccinimide and a coupling reagent such as, but not limited to, EDC ((1-(3-dimethylaminopropyl)-3-ethylcarbodiimide hydrochloride). The reaction is typically performed at ambient temperature in a solvent such as, but not limited to N,N-dimethylformamide.

An amine of formula (3), wherein R¹ and R² are as defined herein, and which can be prepared as described in Scheme 1, can be treated with an acid, such as hydrochloric acid, in a solvent, such as but not limited to dioxane, to provide compounds of formula (11).

Compounds of formula (10) can be coupled with a compound of formula (11) to provide compounds of formula (12). The reaction is typically performed at ambient temperature in a solvent such as, but not limited to N,N-dimethylformamide. Compounds of formula (12) can be converted to compounds of Formula (VII) by coupling with a primary or secondary amine of formula (13), wherein R n is as described herein, in the presence of a base such as triethylamine and coupling agent such as HATU or EDC. The reaction is typically performed at ambient temperature in a solvent such as, but not limited N,N-dimethylformamide.

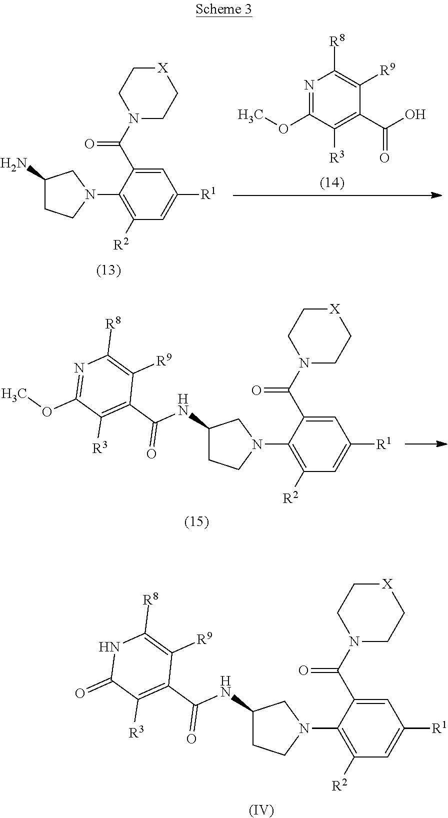

As shown in Scheme 3, compounds of formula (14), wherein R³, R⁸ and R⁹ are as described herein, can be reacted with compounds of formula (13), wherein R¹, R² and X are as described herein, after treatment with a coupling agent such as DMTMM (4-(4,6-dimethoxy-1,3,5-triazin-2-yl)-4-methylmorpholinium chloride, to provide compounds of formula (15). The reaction is typically performed at ambient temperature in a solvent such as, but not limited to N,N-dimethylformamide. Compounds of formula (15) can be treated with a reagent such as trimethylsilyl iodide, formed in situ from trimethylsilyl chloride and potassium iodide in acetonitrile, to provide compounds of formula (IV). The reaction is typically performed at an elevated temperature in a solvent such as, but not limited to acetonitrile.

### Example 1—Synthesis of Compound (2) (R)—N-(1-(4-Cyano-2-(Morpholine-4-Carbonyl)-6-(Trifluoromethyl)Phenyl)Pyrrolidin-3-Yl)-8-Fluoro-2-Oxo-1,2-Dihydroquinoline-4-Carboxamide

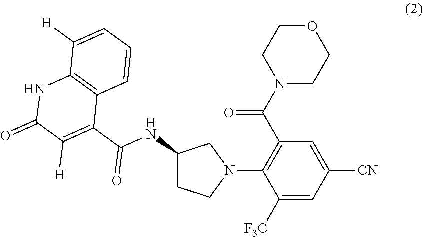 [SMILES: N#Cc1cc(C(=O)N2CCOCC2)c(N2CC[C@@H](NC(=O)c3cc(=O)[nH]c4ccccc34)C2)c(C(F)(F)F)c1]

Compound 2A— Synthesis of 2,2,6,6-tetramethylpiperidinylmagnesium chloride lithium chloride complex solution in tetrahydrofuran

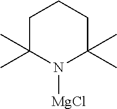

This procedure was adapted from dos Santos, et al, Org. Lett. 2021, v23, 19, 7396-7400. A 500 mL 24/40 round bottom single neck flask containing 1.3 M isopropylmagnesium chloride lithium chloride complex solution in tetrahydrofuran (200 mL, 260 mmol) under nitrogen atmosphere was treated with 2,2,6,6-tetramethylpiperidine (46.3 mL, 273 mmol), added slowly over 10 minutes. The flask was covered in aluminum foil and the mixture was stirred for 48 hours. The resulting solution of 2,2,6,6-tetramethylpiperidinylmagnesium chloride lithium chloride complex (2A) was not titrated and was assumed to be 1.1 M in tetrahydrofuran.

Compound 2B— Synthesis of 5-cyano-2-fluoro-3-(trifluoromethyl)benzoic acid

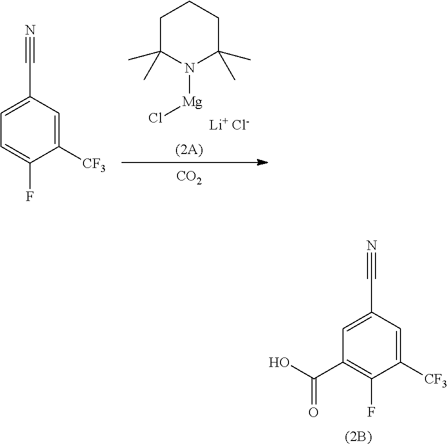

In a 3 L single neck round bottom flask, a solution of 4-fluoro-3-(trifluoromethyl)benzonitrile (50 g, 264 mmol) in anhydrous tetrahydrofuran (1 L) under nitrogen atmosphere was cooled to −40° C. in a dry ice/acetone bath, and treated slowly with 1.1 M 2,2,6,6-tetramethylpiperidinylmagnesium chloride lithium chloride complex solution in tetrahydrofuran (264 mL, 291 mmol; Compound 2A) over about 15 minutes keeping the internal temperature below −35° C. during the addition. The mixture was allowed to warm to −17° C., and then maintained between −17° C. and −16° C. for 15 minutes. The mixture was cooled to −78° C. and treated with a stream of carbon dioxide gas. The stream of carbon dioxide was generated using a 1 L 24/40 one neck round bottom flask containing dry ice (˜200 g). A 24/40 septum with a cannula (˜13-gauge inner diameter, ˜85 mm long) through it was placed on the flask containing the dry ice, and the other end of the cannula was placed into the reaction mixture. During the first 5 minutes of the slow addition of carbon dioxide, the internal temperature of the reaction warmed to about −75° C. The 1 L flask containing the dry ice was then gently warmed to increase the flow rate of CO₂, and the reaction mixture gradually warmed to −70° C. over 10 minutes. Carbon dioxide gas addition was maintained for an additional 5 minutes (20 minutes total) (internal reaction temperature was −69° C. at this point). The reaction mixture was removed from the dry ice bath then allowed to warm. When the reaction reached −10° C., the mixture was poured in portions into a cooled (— 5° C.) swirled 6 L Erlenmeyer flask containing heptane (2.5 L) and 1 M aqueous HCl (750 mL) cooled in an ice bath. The resulting mixture was transferred to a 5 L separatory funnel. Minimal amounts of 1 M aqueous HCl followed by ethyl acetate were used to rinse the residue in the flasks into the separatory funnel. This mixture was shaken for several minutes to dissolve materials that were present. The organic layer was isolated and washed with more 1 M aqueous HCl (750 mL). The organic layer was then extracted with a 1:1 mixture of saturated aqueous sodium bicarbonate (500 mL) and water (500 mL). This mixture was shaken for a few minutes. This lower aqueous layer was immediately added slowly (over ˜5 minutes) to a swirled 4 L Erlenmeyer flask cooled in an ice bath containing ethyl acetate (1.5 L) and 3 M aqueous HCl (350 mL). This ethyl acetate layer was isolated, washed twice with brine (2×50 mL), dried (magnesium sulfate), and filtered. The filtrate was concentrated to provide the title compound (2B). ¹H NMR (600 MHz, CDCl₃) δ ppm 8.55 (dd, J=6.0, 2.3 Hz, 1H), 8.18-8.13 (m, 1H), 7.28 (bs, 1H).

Compound 2C— Synthesis of (R)-2-(3-((tert-butoxycarbonyl)amino)pyrrolidin-1-yl)-5-cyano-3-(trifluoromethyl)benzoic acid

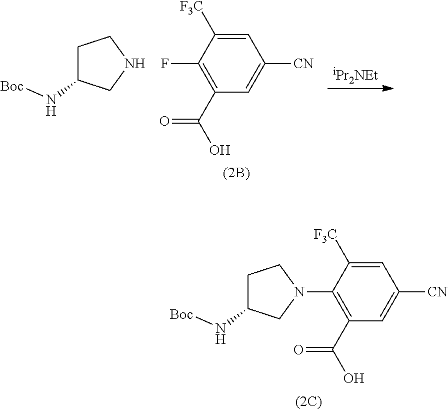

A solution of Compound 2B (28.0 g, 120 mmol), (R)-tert-butyl pyrrolidin-3-ylcarbamate (23.9 g, 128 mmol) and diisopropylethylamine (52.3 mL, 300 mmol) in anhydrous acetonitrile (190 mL) was heated at 60° C. overnight, brought to room temperature and concentrated to remove most of the solvent. The remaining mixture was partitioned between methyl tert-butyl ether (200 mL) and 1 M aqueous citric acid (200 mL) and the separated aqueous phase was extracted twice with methyl tert-butyl ether (100 mL and 50 mL). The combined organic phases were washed with water, then brine, dried (Na₂SO₄), and filtered. The filtrate was concentrated to provide the title compound (2C) which was used in the next step without further purification. MS (APCI, M+H⁺) m/z 400; ¹H NMR (400 MHz, DMSO-d₆) δ ppm 13.6 (b s, 1H), 8.21 (d, J=2.1 Hz, 1H), 8.13 (d, J=2.1 Hz, 1H), 7.09 (d, J=6.7 Hz, 1H), 4.12-4.02 (m, 1H), 3.54-3.46 (m, 1H), 3.44-3.3 (m, 2H), 3.15 (dd, J=9.7, 5.5 Hz, 1H), 2.16-2.06 (m, 1H), 1.88-1.79 (m, 1H), 1.37 (s, 9H).

Compound 2D— Synthesis of tert-butyl (R)-(1-(4-cyano-2-(morpholine-4-carbonyl)-6-(trifluoromethyl)phenyl)pyrrolidin-3-yl)carbamate

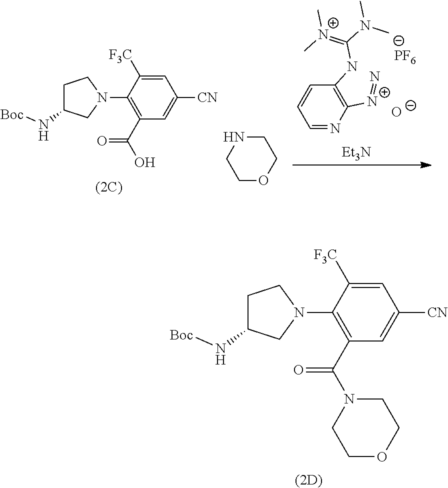

A solution of Compound 2C (≤120 mmol) and morpholine (14.0 mL, 160 mmol) in anhydrous N,N-dimethylformamide (300 mL) was treated with HATU (1-[bis(dimethylamino)methylene]-1H-1,2,3-triazolo[4,5-b]pyridinium 3-oxid hexafluorophosphate) (54.75 g, 144 mmol) over ten minutes, keeping the internal temperature below 28° C. with the aid of a water bath, followed by triethylamine (42 mL, 301 mmol) over twelve minutes, slower at first, keeping the internal temperature below 30° C. After forty minutes, the reaction mixture was poured into water (1500 mL) and extracted thrice with methyl tert-butyl ether (500, 250, and 250 mL). The combined extracts were washed with water (500 mL) then brine, dried (Na₂SO₄), and filtered. The filtrate was concentrated and the title compound was used without further purification. MS (APCI, M+H⁺) m/z 469; ¹H NMR (400 MHz, DMSO-d₆) δ ppm 8.17-8.13 (m, 1H), 7.92-7.88 (m, 1H), 7.15-6.97 (m, 1H), 4.06-3.91 (m, 1H), 3.75-3.20 (m, 11H), 3.17-3.04 (m, 1H), 2.12-2.02 (m, 1H), 1.87-1.76 (m, 1H), 1.38 (s, 9H).

Compound 2E— Synthesis of (R)-4-(3-aminopyrrolidin-1-yl)-3-(morpholine-4-carbonyl)-5-(trifluoromethyl)benzonitrile hydrochloride

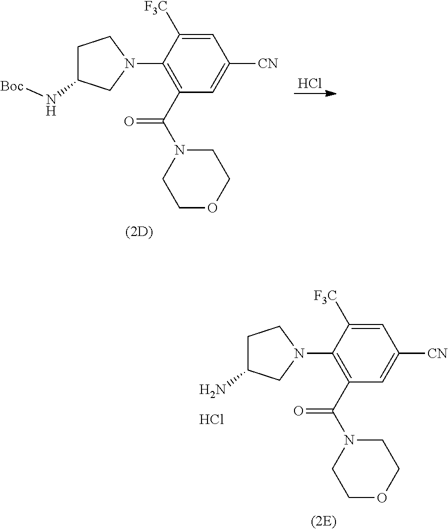

Compound 2D (≤120 mmol) was dissolved into anhydrous acetonitrile (300 mL), treated with 3 M HCl in cyclopentyl methyl ether (120 mL, 360 mmol) and stirred one hour before being placed in the refrigerator overnight. Then the reaction mixture was concentrated to provide the title compound (2E) which was used without further purification. MS (APCI, M-41+) m/z 369.

Compound 2F— Synthesis of 8-fluoro-2-oxo-1,2-dihydroquinoline-4-carboxylic acid

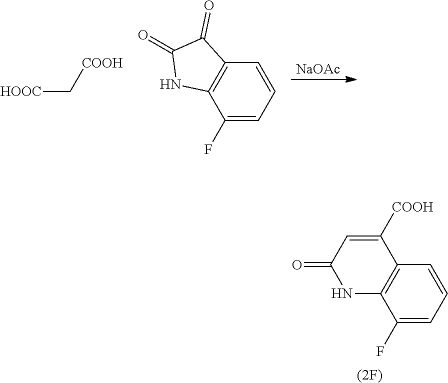

A suspension of 7-fluoroindoline-2,3-dione (55 g, 333 mmol), malonic acid (41.6 g, 400 mmol) and sodium acetate (68.3 g, 833 mmol) in acetic acid (500 mL) was heated at 112° C. overnight. The reaction mixture was cooled to room temperature and poured into cold 0.4 M aqueous HCl (2200 mL). The precipitate was collected by filtration and rinsed thoroughly with ice-cold water (˜250 mL) followed by methyl tort-butyl ether (˜100 mL) and then concentrated twice from acetonitrile with high vacuum. The materials were largely dissolved into 1 M aqueous NaOH (370 mL) and filtered through diatomaceous earth with a 0.1 M aqueous NaOH (50 mL) rinse. Then the filtrate was washed thrice with dichloromethane (3×200 mL) which removed the color. After this aqueous layer was filtered again through diatomaceous earth, it was acidified by the dropwise addition of concentrated aqueous HCl (33 mL, ˜0.4 moles). The material was collected by filtration. After prolonged drying under heat and vacuum, the material was treated with water (1 L) and the mixture was made acidic by the addition of a small amount of 1 M aqueous HCl. The suspension was heated to 80° C. and then allowed to slowly cool to room temperature. The resulting material was collected by filtration, washed with 0.01 M aqueous HCl (150 mL) and dried under vacuum at 80° C. to provide the title compound (2F). ¹H NMR (500 MHz, DMSO-d₆) δ ppm 14.00 (bs, 1H), 12.07 (bs, 1H), 8.00 (dd, J=8.2, 1.2 Hz, 1H), 7.49 (ddd, J=11.0, 8.1, 1.2 Hz, 1H), 7.23 (ddd, J=8.2, 8.1, 5.2 Hz, 1H), 6.95 (s, 1H); ¹³C NMR (101 MHz, DMSO-d 6, 90° C.) δ ppm 165.75-165.73 (m), 160.30, 148.75 (d, J=246.0 Hz), 140.85-140.80 (m), 128.11 (d, J=13.7 Hz), 123.85, 121.60-121.53 (m), 121.53-121.43 (m), 117.70-117.65 (m), 115.33 (d, J=17.2 Hz); ¹⁹F NMR (376 MHz, DMSO-d 6, 90° C.) δ ppm −130.47 (dd, J=10.9, 5.3 Hz); MS (APCI, M+H⁺) m/z 208.

Compound 2G— Synthesis of (R)—N-(1-(4-cyano-2-(morpholine-4-carbonyl)-6-(trifluoromethyl)phenyl)pyrrolidin-3-yl)-8-fluoro-2-oxo-1,2-dihydroquinoline-4-carboxamide (2)

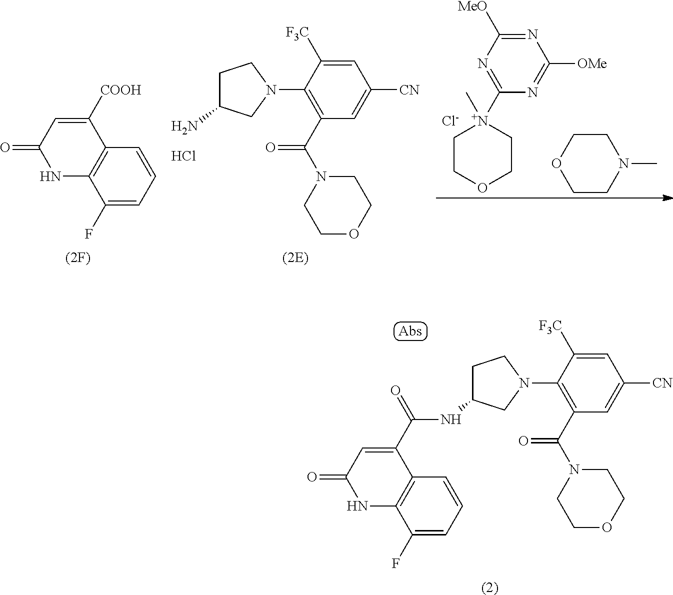

To a mixture of Compound 2F (29.84 g, 144 mmol) in anhydrous N,N-dimethylformamide (360 mL) was added DMTMM (4-(4,6-dimethoxy-1,3,5-triazin-2-yl)-4-methylmorpholinium chloride) (43.17 g, 156 mmol) over twelve minutes at room temperature. After the suspension had been stirred forty minutes, it was added over eight minutes to a suspension of Compound 2E (≤120 mmol) and N-methylmorpholine (16 mL, 146 mmol) in N,N-dimethylformamide (120 mL) with a N,N-dimethylformamide (20 mL) rinse. After forty minutes, the reaction mixture was added to rapidly stirred 0.1 M aqueous K₂HPO₄ (2.5 L) and extracted four times with 4:1 isopropyl acetate/heptanes then once with isopropyl acetate alone. The product, which had begun to precipitate out from the combined extracts, was separated by decantation and filtration, then washed with dichloromethane. The remaining aqueous phase was extracted twice more with isopropyl acetate and all the organic extracts were combined, then washed with additional 0.1 M aqueous K₂HPO₄ followed by water, dried (Na₂SO₄), and filtered. The filtrate was concentrated with the dichloromethane wash of the material collected above. The residue was concentrated, dissolved in acetonitrile/CH₂C₁₂, filtered, and purified by chromatography on silica (20 to 100% acetonitrile/CH₂C₁₂). The collected fractions were concentrated to a small volume, and stirred in ethyl acetate overnight.

The suspension was heated at 70° C. for twenty minutes, then allowed to slowly cool to room temperature. Methyl tert-butyl ether was stirred in, the suspension was cooled to 0° C., and the purified product was collected by filtration with rinses of 1:1 ethyl acetate/methyl tert-butyl ether followed by methyl tert-butyl ether before being dried under vacuum with heat. The material obtained previously from the early extracts were also stirred in ethyl acetate, heated at 70° C., then allowed to slowly cool to room temperature. Methyl tert-butyl ether was stirred in, the suspension was cooled to 0° C., and the purified product was collected by filtration with rinses of 1:1 ethyl acetate/methyl tert-butyl ether rinse followed by methyl tert-butyl ether. The material was dried overnight under vacuum to provide the title compound (2). ¹H NMR (500 MHz, DMSO-d₆) δ ppm 11.96 (s, 1H), 9.03-8.84 (m, 1H), 8.21-8.18 (m, 1H), 7.98-7.93 (m, 1H), 7.56-7.50 (m, 1H), 7.49-7.43 (m, 1H), 7.21-7.15 (m, 1H), 6.66-6.59 (m, 1H), 4.51-4.40 (m, 1H), 3.73-3.55 (m, 6H), 3.54-3.22 (m, 6H), 2.29-2.18 (m, 1H), 2.07-1.95 (m, 1H); ¹H NMR (400 MHz, DMSO-d₆, 90° C.) δ ppm 11.47 (bs, 1H), 8.77-8.47 (m, 1H), 8.09 (d, J=2.1 Hz, 1H), 7.88 (d, J=2.1 Hz, 1H), 7.54 (dd, J=8.1, 1.2 Hz, 1H), 7.39 (ddd, J=11.0, 8.1, 1.2 Hz, 1H), 7.15 (ddd, J=8.1, 8.1, 5.1 Hz, 1H), 6.60 (s, 1H), 4.54-4.43 (m, 1H), 3.74-3.20 (m, 12H), 2.31-2.21 (m, 1H), 2.06-1.96 (m, 1H); ¹³C NMR (101 MHz, DMSO-d₆, 90° C.) δ ppm 166.61, 165.97, 161.31, 149.59 (d, J=246.3 Hz), 148.95, 146.10-146.03 (m), 136.00, 135.65, 133.13 (q, J=6.1 Hz), 128.84-128.63 (m), 123.76 (q, J=273.7 Hz), 122.19, 122.16, 122.12, 121.60, 118.99-118.91 (m), 117.75, 116.17 (d, J=17.3 Hz), 105.57, 66.04, 57.95, 51.06, 50.35, 47.74, 42.35, 31.54; ¹⁹F NMR (376 MHz, DMSO-d₆) δ ppm −57.54-58.10 (m), −130.02 130.15 (m); ¹⁹F NMR (376 MHz, DMSO-d₆, 90° C.) 6 ppm −58.37 58.97 (m), −130.96 (dd, J=11.0, 5.1 Hz). MS (APCI, M+H⁺) m/z 558.

### Example 2—Synthesis of Compound (1)

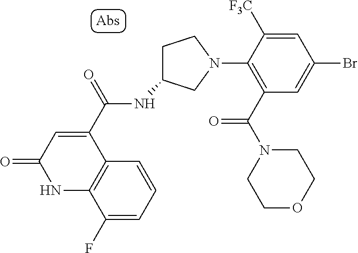 [SMILES: O=C(N[C@@H]1CCN(c2c(C(=O)N3CCOCC3)cc(Br)cc2C(F)(F)F)C1)c1cc(=O)[nH]c2c(F)cccc12]

(R)-N-(1-(4-bromo-2-(morpholine-4-carbonyl)-6-(trifluoromethyl)phenyl)pyrrolidin-3-yl)-8-fluoro-2-oxo-1,2-dihydroquinoline-4-carboxamide (1)

Compound 1A— Synthesis of 5-bromo-2-fluoro-3-(trifluoromethyl)benzoic acid

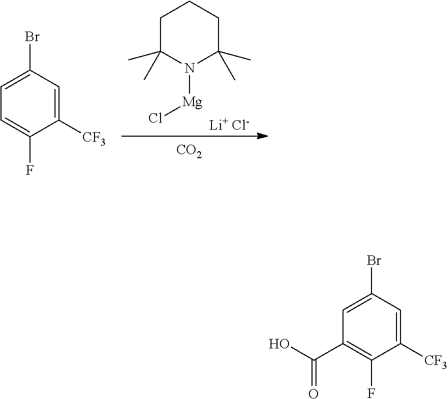

In a 3 L round bottom single neck flask, a solution of 5-bromo-2-fluorobenzotrifluoride (64.2 g, 264 mmol) in tetrahydrofuran (1 L) under nitrogen atmosphere was cooled to −40° C., treated with 1.1 M 2,2,6,6-tetramethylpiperidinylmagnesium chloride lithium chloride complex solution in tetrahydrofuran (264 mL, 290 mmol) over 15 minutes, and then allowed to warm to 0° C. The mixture was stirred at 0° C. for 15 minutes, then cooled to −78° C. The mixture was treated with a stream of carbon dioxide gas. The stream of carbon dioxide was generated using a 1 L 24/40 one neck round bottom flask containing dry ice (˜200 g). A 24/40 septum with a cannula (˜13 gauge inner diameter, ˜85 mm long) through it was placed on the flask containing the dry ice, and the other end of the cannula was placed into the reaction mixture. During the first 5 minutes of the slow addition of CO₂, the internal temperature of the reaction gently warmed to about −75° C. The 1 L flask containing the dry ice was then gently warmed to increase the flow rate of CO₂, and the reaction mixture gradually warmed to −70° C. over 10 minutes. CO₂ addition was maintained for an additional 5 minutes (20 minutes total) (internal reaction temperature was −69° C. at this point). The reaction mixture was removed from the dry ice bath and allowed to warm. When the reaction reached −10° C., the mixture was poured in portions into a cooled (˜5° C.) swirled 6 L Erlenmeyer flask containing heptane (2.5 L) and 1 M aqueous HCl (750 mL) cooled in an ice bath. The resulting mixture was transferred to a 5 L separatory funnel. Minimal amounts of 1 M aqueous HCl followed by ethyl acetate were used to rinse the residue in the flasks into the separatory funnel. This mixture in the separatory funnel was shaken for several minutes. The organic layer was isolated and washed with more 1 M aqueous HCl (750 mL). The organic layer was then extracted with a 1:1 mixture of saturated aqueous NaHCO₃ (500 mL) and water (500 mL). This mixture in the separatory funnel was shaken for a few minutes. This lower aqueous layer was then added over about 5 minutes to a swirled 4 L Erlenmeyer flask cooled in an ice bath containing ethyl acetate (1.5 L) and 3 M aqueous HCl (350 mL). The ethyl acetate layer was isolated, washed twice with brine (2×50 mL), dried (magnesium sulfate), and filtered. The filtrate was concentrated to provide the title compound (1A). ¹H NMR (500 MHz, CDCl₃) δ ppm 8.34 (dd, J=5.8, 2.6 Hz, 1H), 7.97 (dd, J=5.6, 2.6 Hz, 1H). ¹H NMR (600 MHz, DMSO-d₆) δ ppm 14.02 (s, 1H), 8.26 (dd, J=6.0, 2.6 Hz, 1H), 8.23 (dd, J=5.7, 2.6 Hz, 1H).

Compound 1B—(R)-5-bromo-2-(3-((tert-butoxycarbonyl)amino)pyrrolidin-1-yl)-3-(trifluoromethyl)benzoic acid

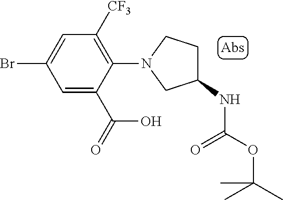 [SMILES: CC(C)(C)OC(=O)N[C@@H]1CCN(c2c(C(=O)O)cc(Br)cc2C(F)(F)F)C1]

Option 1, K₂CO₃ method

In a 3-neck 1 L round bottom flask equipped with a mechanical stirrer, a mixture of Compound 1A (40 g, 139 mmol), (R)-3-(Boc-amino)pyrrolidine (31.1 g, 167 mmol) and K₂CO₃ (77 g, 557 mmol) in dimethyl sulfoxide (400 mL) under nitrogen atmosphere was stirred mechanically at 125° C. for 20 hours. The mixture was cooled and became quite thick. The mixture was added portion wise to a swirled 4 L Erlenmeyer flask containing 1 M aqueous HCl (1 L) and a mixture of heptane (1 L) and ethyl acetate (200 mL) that was cooled in an ice bath. The mixture was transferred to a separatory funnel, shaken, and the layers were separated. The aqueous layer was then extracted with a mixture heptane (500 mL) and ethyl acetate (100 mL). The organics layers were combined, washed twice with 0.01 M aqueous HCl (2×400 mL), washed with brine (50 mL), dried (magnesium sulfate), and filtered. The filtrate was concentrated to provide a mixture of the title compound (1B) and 5-bromo-2-fluoro-3-(trifluoromethyl)benzoic acid. ¹H NMR (400 MHz, DMSO-d₆) δ ppm 13.78 (s, 1H), 8.05 (d, J=2.5 Hz, 1H), 7.97 (d, J=2.5 Hz, 1H), 6.92 (d, J=6.9 Hz, 1H), 4.09 (q, J=7.1 Hz, 1H), 3.32 (t, J=7.3 Hz, 1H), 3.22-3.13 (m, 2H), 3.00-2.94 (m, 1H), 2.14-2.04 (m, 1H), 1.81 (dq, J=12.0, 8.0 Hz, 1H), 1.38 (s, 9H); LC/MS (ESI+) m/z 453,455 (M+H)⁺.

Compound 1C— Synthesis of 2,2,6,6-tetramethylpiperidin-1-ium (R)-5-bromo-2-(3-((tert-butoxycarbonyl)amino)pyrrolidin-1-yl)-3-(trifluoromethyl)benzoate

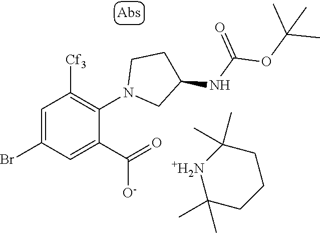

Option 2, TMP method

A mixture of (R)-3-(Boc-amino)pyrrolidine (43.8 g, 235 mmol), Compound 1A (45 g, 157 mmol), and 2,2,6,6-tetramethylpiperidine (66.4 g, 470 mmol) in anhydrous 1-methyl-2-pyrrolidinone (180 mL) was heated to 100° C. under nitrogen atmosphere overnight. A small aliquot of the reaction mixture was transferred to a test tube and diluted with water. This aliquot/water mixture was agitated intermittently with a spatula for ˜45 minutes and a precipitate formed. The reaction mixture was then removed from the heating mantle and allowed to cool. When the internal temperature had cooled to 34° C., the mixture was treated over ˜9 minutes with water (90 mL). The mixture was stirred for 5 minutes and then treated with the precipitate from the aliquot and the mixture was stirred at room temperature for 1 hour. The mixture was then heated to 55° C. and treated with more water (270 mL) over 35 minutes, then stirred at room temperature for 4 hours. The material was collected by filtration in a Buchner funnel, washed with water (3×225 mL), and dried in the Buchner funnel with vacuum flowing for 1 hour, then dried overnight under vacuum at 65° C. to provide the title compound (1C). ¹H NMR (400 MHz, DMSO-d₆) δ ppm 8.43 (s, 2H), 7.66 (d, J=2.5 Hz, 1H), 7.61 (d, J=2.5 Hz, 1H), 7.29 (d, J=7.7 Hz, 1H), 4.12-4.04 (m, 1H), 3.45-3.38 (m, 2H), 3.12-3.04 (m, 2H), 2.00-1.90 (m, 1H), 1.73-1.63 (m, 3H), 1.58-1.52 (m, 4H), 1.39 (s, 9H), 1.35 (s, 12H); ¹H NMR (400 MHz, DMSO-d₆, 90° C.) δ ppm 7.72 (d, J=2.6 Hz, 1H), 7.59 (d, J=2.6 Hz, 1H), 6.90 (s, 1H), 4.14-4.06 (m, 1H), 3.46 (dd, J=8.7, 6.6 Hz, 1H), 3.41 (q, J=7.5 Hz, 1H), 3.15-3.09 (m, 2H), 2.01 (dtd, J=12.0, 7.8, 6.4 Hz, 1H), 1.79-1.65 (m, 3H), 1.58-1.53 (m, 5H), 1.41 (s, 9H), 1.34 (s, 12H); LC/MS (ESI+) m/z 453,455 (M+H)⁺.

Compound 1D— Synthesis of tert-butyl (R)-(1-(4-bromo-2-(morpholine-4-carbonyl)-6-(trifluoromethyl)phenyl)pyrrolidin-3-yl)carbamate

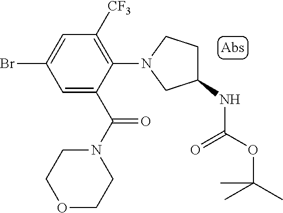 [SMILES: CC(C)(C)OC(=O)N[C@@H]1CCN(c2c(C(=O)N3CCOCC3)cc(Br)cc2C(F)(F)F)C1]

Option 1, from Compound 1B

A solution of Compound 1B (63.0 g, 139 mmol) in N,N-dimethylformamide (340 mL) was cooled to 0° C., treated with morpholine (14.53 mL, 167 mmol) (mild exotherm from 2° C. to 6° C.), cooled back to 2° C., treated in 3 portions with 1-[bis(dimethylamino)methylene]-1H-1,2,3-triazolo[4,5-b]pyridinium 3-oxid hexafluorophosphate (68.7 g, 181 mmol) (mild exotherm to 4° C. after each portion added), and treated with triethylamine (48.4 mL, 348 mmol) slowly so that the reaction temperature did not exotherm above 8° C. The reaction was stirred at room temperature for 45 minutes. The mixture was diluted with a solution of heptanes (500 mL) and ethyl acetate (100 mL) and cooled to 0° C. This mixture was added to a swirled 4 L Erlenmeyer flask containing 1.6 L of water near 0° C. in an ice bath. The mixture was transferred to a 4 L separatory funnel and the layers were separated. The aqueous layer was extracted with a mixture of heptanes (500 mL) and ethyl acetate (100 mL). The organic layers were combined, washed with water twice (2×400 mL), washed with brine (50 mL), dried (magnesium sulfate), and filtered. The filtrate was concentrated to approximately 100 mL volume. A spatula was used to scrape the material from the sides of the flask.

The material was collected by filtration, and washed with the 40:1 heptanes:ethyl acetate to provide material that was dried under vacuum at 50° C. overnight to provide the desired product, (R)-tert-butyl (1-(4-bromo-2-(morpholine-4-carbonyl)-6-(trifluoromethyl)phenyl)pyrrolidin-3-yl)carbamate contaminated with (5-bromo-2-fluoro-3-(trifluoromethyl)phenyl)(morpholino)methanone. The product from this reaction was combined with the same product from a separate run and dissolved in ethyl acetate (300 mL). This solution was diluted with heptane and purified by chromatography on silica gel (200 g silica gel, 24 cm×4.5 cm column) eluting with 1:1 heptane: ethyl acetate. The fractions containing the product were concentrated. The residue was treated with heptanes (350 mL) and the flask was manually swirled for ˜15 minutes to dissolve the majority of the material. The mixture was allowed to stand at room temperature for 15 minutes. The material was scraped from the sides of the flask. The material was collected by filtration and washed with heptanes. The material was dried under vacuum at 55° C. for about 1.5 hours to provide the title compound (1D). ¹H NMR (600 MHz, DMSO-d₆) δ ppm 7.86 (m, 1H), 7.81 (d, J=2.4 Hz, 0.5H), 7.79 (d, J=2.4 Hz, 0.5H), 6.98 (d, J=6.7 Hz, 0.5H), 6.82 (d, J=6.8 Hz, 0.5H), 3.96 (m, 1H), 3.77-3.54 (m, 3H), 3.46 (m, 1H), 3.36-3.13 (s, 4.5H), 3.10 (q, J=7.5 Hz, 0.5H), 2.97 (dd, J=8.9, 5.9 Hz, 0.5H), 2.90 (t, J=7.4 Hz, 0.5H), 2.08-1.99 (m, 1H), 1.82-1.67 (m, 1H), 1.38 (s, 4.5H), 1.38 (s, 4.5H); LC/MS (ESI+) m/z 522,524 (M+H)⁺.

Option 2, from Compound 1C

A solution of Compound 1C (60 g, 101 mmol) in N,N-dimethylformamide (240 mL) was cooled to 0° C., treated slowly with morpholine (10.55 mL, 121 mmol) (mild exotherm to 3° C.), treated in portions with 1-[bis(dimethylamino)methylene]-1H-1,2,3-triazolo[4,5-b]pyridinium 3-oxid hexafluorophosphate (49.9 g, 131 mmol) over 25 minutes (mild exotherm between 2.7 and 3.3° C. during addition), and treated with triethylamine (35.2 mL, 252 mmol) slowly so that the reaction temperature did not exotherm above 4° C. The reaction mixture was removed from the cooling bath and stirred at room temperature for 20 minutes. The reaction mixture was diluted with ethyl acetate (45 mL), cooled in an ice bath to ˜3 C, and transferred to a cooled (ice bath) swirled 4 L Erlenmeyer flask containing ethyl acetate (45 mL), heptanes (450 mL) and water (1.2 L). This mixture was transferred to a separatory funnel. After the mixture was shaken, the layers were separated, and the aqueous layer was extracted with 5:1 heptanes:ethyl acetate (500 mL).

The combined organic layers were washed twice with water (2×300 mL), washed with brine, dried (magnesium sulfate), and filtered. The filtrate was concentrated to a volume of approximately 200 mL. The material that was stuck on the sides of the flask was scraped off with a spatula. The material was isolated by filtration, washed with cool (˜5° C.) 40:1 heptane:ethyl acetate, dried under a vacuum flow for 30 minutes, then dried overnight under vacuum in a drying oven at 65° C. to provide the title compound (1D). ¹H NMR (600 MHz, DMSO-d₆) δ ppm 7.86 (m, 1H), 7.81 (d, J=2.4 Hz, 0.5H), 7.79 (d, J=2.4 Hz, 0.5H), 6.98 (d, J=6.7 Hz, 0.5H), 6.82 (d, J=6.8 Hz, 0.5H), 3.96 (m, 1H), 3.77-3.54 (m, 3H), 3.46 (m, 1H), 3.36-3.13 (s, 4.5H), 3.10 (q, J=7.5 Hz, 0.5H), 2.97 (dd, J=8.9, 5.9 Hz, 0.5H), 2.90 (t, J=7.4 Hz, 0.5H), 2.08-1.99 (m, 1H), 1.82-1.67 (m, 1H), 1.38 (s, 4.5H), 1.38 (s, 4.5H); LC/MS (ESI+) m/z 522,524 (M+H)⁺.

Compound 1E— Synthesis of (R)-(2-(3-aminopyrrolidin-1-yl)-5-bromo-3-(trifluoromethyl)phenyl)(morpholino)methanone hydrochloride

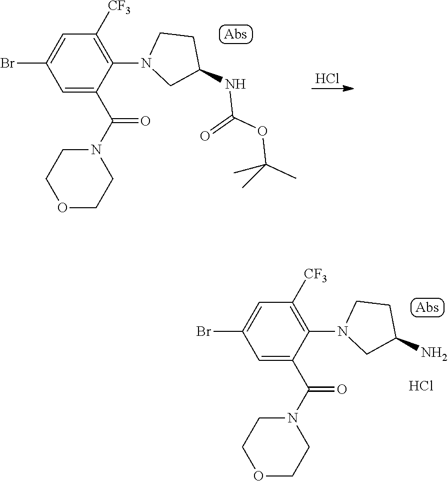 [SMILES: CC(C)(C)OC(=O)N[C@@H]1CCN(c2c(C(=O)N3CCOCC3)cc(Br)cc2C(F)(F)F)C1 ; Cl ; N[C@@H]1CCN(c2c(C(=O)N3CCOCC3)cc(Br)cc2C(F)(F)F)C1 ; Cl]

A suspension of Compound 1D (79.2 g, 152 mmol) in acetonitrile (160 mL, anhydrous) was treated with 3 M HCl in cyclopentyl methyl ether (152 mL, 455 mmol). The mixture was stirred at room temperature for overnight. The mixture was then concentrated on the rotovap to provide the title compound (1E). ¹H NMR (600 MHz, DMSO-d₆) δ ppm 8.51-8.39 (m, 3H), 7.89 (apparent d, J=2.4 Hz, 1H), 7.86 (apparent dd, J=4.3, 2.4 Hz, 1H), 3.78-3.35 (m, 8H), 3.32-3.07 (m, 5H), 2.27-2.19 (m, 1H), 1.98-1.85 (m, 1H); LC/MS (ESI+) m/z 422,424 (M+H)⁺.

Compound 1F— Synthesis of (R)—N-(1-(4-bromo-2-(morpholine-4-carbonyl)-6-(trifluoromethyl)phenyl)pyrrolidin-3-yl)-8-fluoro-2-oxo-1,2-dihydroquinoline-4-carboxamide

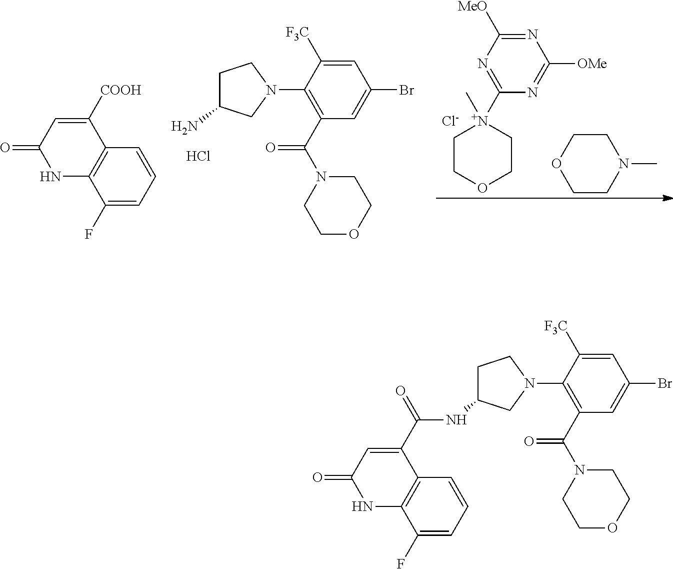

A mixture of 8-fluoro-2-oxo-1,2-dihydroquinoline-4-carboxylic acid (7.66 g, 37.0 mmol; Compound 2F) and previously crushed DMTMM (4-(4,6-dimethoxy-1,3,5-triazin-2-yl)-4-methylmorpholinium chloride) (11.07 g, 40.0 mmol) were placed in anhydrous N,N-dimethylformamide (110 mL) and stirred for thirty minutes. Then a solution of Compound 1E (-28.7 mmol) and N-methylmorpholine (4.07 mL, 37.0 mmol) in N,N-dimethylformamide (30 mL) was added with a N,N-dimethylformamide (10 mL) rinse. After forty minutes, the reaction mixture was slowly added to rapidly stirred water (750 mL). The resulting suspension was cooled to 7° C. and the material was collected by filtration with an ice-cold water rinse. Collection flasks were exchanged and the material was rinsed through with dichloromethane. The aqueous phase which separated in the filtrate was extracted with dichloromethane and the combined extracts were washed with dilute brine, dried (Na₂SO₄), concentrated, filtered and chromatographed on silica (40 to 100% ethyl acetate/dichloromethane). The appropriate fractions were combined and concentrated, then taken up into acetonitrile. The suspension was diluted with ethyl acetate and the material was collected by filtration with an ethyl acetate rinse.

The filtrate precipitated a considerable portion of additional material and these too were collected by filtration but washed with 1:1 ethyl acetate/methyl tert-butyl ether. The material was placed under vacuum overnight, then crushed and dried further under vacuum with gentle heating to give the title compound. Eventually, an additional amount of material was obtained from the earlier filtrate and washed with 100% methyl tert-butyl ether, then dried under vacuum with gentle heating to give additional title compound (1). ¹H NMR (400 MHz, DMSO-d₆) δ ppm 11.95 (s, 1H), 8.98-8.78 (m, 1H), 7.91-7.87 (m, 1H), 7.86-7.82 (m, 1H), 7.56-7.50 (m, 1H), 7.50-7.43 (m, 1H), 7.22-7.15 (m, 1H), 6.66-6.59 (m, 1H), 4.48-4.37 (m, 1H), 3.74-3.42 (m, 7H), 3.32-3.06 (m, 5H), 2.28-2.14 (m, 1H), 2.01-1.85 (m, 1H); ¹H NMR (400 MHz, DMSO-d 6, 120° C.) δ ppm 11.24 (bs, 1H), 8.37 (s, 1H), 7.79 (d, J=2.4 Hz, 1H), 7.67 (d, J=2.4 Hz, 1H), 7.59-7.52 (m, 1H), 7.39-7.31 (m, 1H), 7.17-7.10 (m, 1H), 6.59 (s, 1H), 4.52-4.41 (m, 1H), 3.70-3.20 (m, 11H), 3.19-3.12 (m, 1H), 2.28-2.18 (m, 1H), 2.01-1.91 (m, 1H); ¹³C NMR (101 MHz, DMSO-d₆, 90° C.) δ ppm 165.40, 164.92, 160.40, 148.65 (d, J=246.3 Hz), 145.32-145.24 (m), 143.44, 139.57, 134.05, 130.85 (q, J=29.5 Hz), 130.08 (q, J=5.8 Hz), 127.91-127.69 (m), 122.58 (q, J=274.1 Hz), 121.30-121.22 (m), 121.24-121.16 (m), 120.59, 118.10-118.02 (m), 116.72, 115.21 (d, J=17.3 Hz), 65.15, 57.22, 50.36, 49.59, 46.82, 41.35, 30.91; ¹⁹F NMR (376 MHz, DMSO-d₆, 90° C.) δ ppm −60.16 (s), −131.01 (dd, J=11.0, 5.2 Hz); MS (ESI, M+Et)m/z 611/613.

### Example 3—Synthesis of Compound (3)

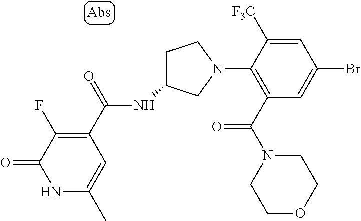 [SMILES: Cc1cc(C(=O)N[C@@H]2CCN(c3c(C(=O)N4CCOCC4)cc(Br)cc3C(F)(F)F)C2)c(F)c(=O)[nH]1]

(R)—N-(1-(4-bromo-2-(morphohne-4-carbonyl)-6-(trifluoromethyl)phenyl)pyrrolidin-3-yl)-3-fluoro-6-methyl-2-oxo-1,2-dihydropyridine-4-carboxamide

Compound 3A—Synthesis of (R)—N-(1-(4-bromo-2-(morpholine-4-carbonyl)-6-(trifluoromethyl)phenyl)pyrrolidin-3-yl)-3-fluoro-2-methoxy-6-methylisonicotinamide

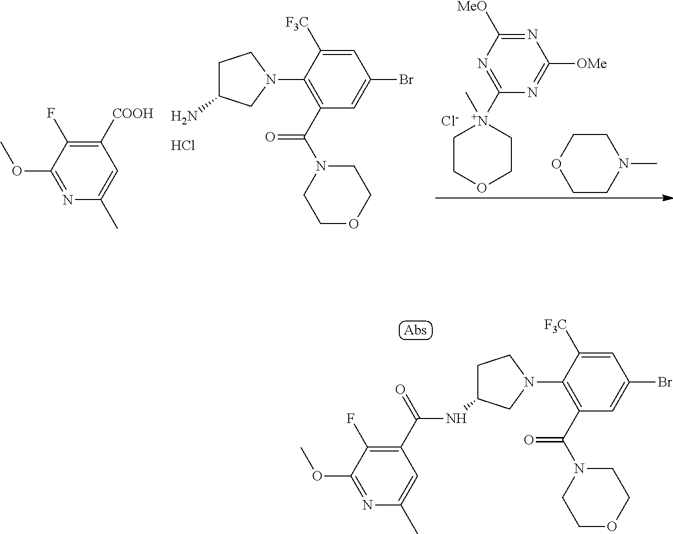

A mixture of 3-fluoro-2-methoxy-6-methylisonicotinic acid (773 mg, 4.17 mmol) and 4-(4,6-dimethoxy-1,3,5-triazin-2-yl)-4-methylmorpholin-4-ium chloride (1.252 g, 4.52 mmol) were placed in anhydrous N,N-dimethylformamide (10 mL), sonicated to break up larger particles of the reagents, and stirred for forty minutes at room temperature. The suspension was then transferred with a glass pipet to a solution of the Compound 1E (1.599 g, 3.48 mmol) and N-methylmorpholine (420 μL, 3.82 mmol) in N,N-dimethylformamide (3 mL) with a N,N-dimethylformamide (1 mL) rinse. After twenty minutes, the reaction mixture was added dropwise to rapidly stirred water (70 mL) with an acetonitrile rinse. The resulting suspension was cooled to 5° C. with a water ice bath and the material was collected by filtration with a thorough wash of ice cold water, then dried under vacuum with gentle heating and a nitrogen stream. The material was dissolved in chloroform; there was a very significant water layer. The organic layer was isolated, dried (Na₂SO₄) and filtered through silica-gel first eluting with 70% tert-butyl methyl ether, then eluting with heptanes, then further eluting with 100% tert-butyl methyl ether to provide the title compound. ¹H NMR (400 MHz, DMSO-d₆, 90° C.) δ ppm 8.27 8.16 (m, 1H), 7.82 (d, J=2.4 Hz, 1H), 7.72 (d, J=2.4 Hz, 1H), 6.89 (d, J=3.7 Hz, 1H), 4.46 4.37 (m, 1H), 3.95 (s, 3H), 3.72-3.43 (m, 7H), 3.34-3.16 (m, 4H), 3.11-3.04 (m, 1H), 2.38 (s, 3H), 2.25-2.15 (m, 1H), 1.95-1.85 (m, 1H). LC/MS (ESI+) m/z 589,591 (M+H)⁺.

Compound 3B—Synthesis of (R)—N-(1-(4-bromo-2-(morpholine-4-carbonyl)-6-(trifluoromethyl)phenyl)pyrrolidin-3-yl)-3-fluoro-6-methyl-2-oxo-1,2-dihydropyridine-4-carboxamide (3)

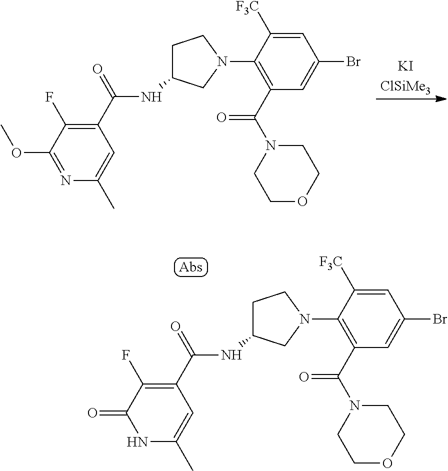

To a suspension of Compound 3A (1.883 g, 3.19 mmol) and potassium iodide (2.65 g, 16.0 mmol) in anhydrous acetonitrile (15 mL) under nitrogen, chlorotrimethylsilane (2.02 mL, 15.9 mmol) was added dropwise. The reaction mixture was heated to 65° C. and kept at 65° C. for four hours. The mixture was then cooled with a water ice bath and quenched with 2 M aqueous K₂CO₃. Aqueous HCl was added to adjust the pH to 7. The mixture was extracted with several times with ethyl acetate. The combined extracts were dried (Na₂SO₄), filtered, and the filtrate was concentrated. The residue was chromatographed on silica gel eluting with a gradient of 20 to 100% ethyl acetate in CH₂C₁₂, then further eluted with 2% methanol in ethyl acetate to provide the title compound (3). ¹H NMR (400 MHz, DMSO-d₆) δ ppm 12.22 (s, 1H), 8.70-8.50 (m, 1H), 7.90-7.86 (m, 1H), 7.85-7.81 (m, 1H), 6.00-5.94 (m, 1H), 4.41-4.29 (m, 1H), 3.75-3.38 (m, 7H), 3.31-3.13 (m, 4H), 3.08-2.97 (m, 1H), 2.23-2.10 (m, 4H), 1.93-1.78 (m, 1H). LC/MS (ESI+) m/z 575,577 (M+H)+. ¹H NMR (400 MHz, DMSO-d₆, 90° C.) δ ppm 11.88 (bs, 1H), 8.19 (bs, 1H), 7.84 (d, J=2.4 Hz, 1H), 7.74 (d, J=2.4 Hz, 1H), 5.96 (d, J=4.7 Hz, 1H), 4.41 (h, J=6.6 Hz, 1H), 3.76-3.04 (m, 12H), 2.26-2.15 (m, 4H), 1.95-1.84 (m, 1H).

### Example 4— Synthesis of Building Blocks

### 3,8-difluoro-2-oxo-1,2-dihydroquinoline-4-carboxylic acid

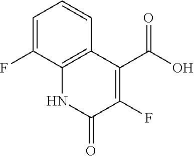 [SMILES: O=C(O)c1c(F)c(=O)[nH]c2c(F)cccc12]

2-Fluoromalonic acid (50 g, 410 mmol) and 7-fluoroindoline-2,3-dione (45.1 g, 273 mmol) were suspended in acetic acid (390 mL) and the suspension was heated to 90° C. for 16 hours. The mixture was cooled to room temperature over 1 hour then the precipitate was collected via filtration. The material was washed with 30 mL of acetic acid then dried to constant weight in the funnel under a stream of air to give the title compound: ¹H NMR (600 MHz, DMSO-d₆) δ ppm 12.61 (s, 1H), 7.49 (ddd, J=11.0, 8.1, 1.2 Hz, 1H), 7.44 (dt, J=8.2, 0.9 Hz, 1H), 7.30 (td, J=8.1, 5.0 Hz, 1H); MS (LSI) m/z=226 (M+H)⁺.

### 3-Fluoro-2-Oxo-1,2-Dihydroquinoline-4-Carboxylic Acid

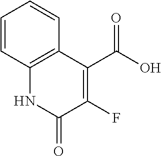 [SMILES: O=C(O)c1c(F)c(=O)[nH]c2ccccc12]

### 4-Bromo-2-Fluoro-3-Oxo-N-Phenylbutanamide

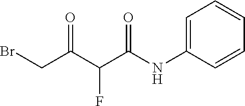 [SMILES: O=C(CBr)C(F)C(=O)Nc1ccccc1]

A mixture of 4-bromo-3-oxo-N-phenylbutanamide (1.5575 g, 4.93 mmol) and (1-chloromethyl-4-fluoro-1,4-diazoniabicyclo[2.2.2]octane bis(tetrafluoroborate)) (1.920 g, 5.42 mmol) in acetonitrile (32.8 mL) was heated to 60° C. for 2 hours. The reaction was cooled to room temperature and concentrated under reduced pressure. The resulting residue was dissolved in dichloromethane and washed with water (2×). The combined organic layers were dried over anhydrous sodium sulfate, filtered, and the filtrate was concentrated under reduced pressure. The resulting residue was purified via flash chromatography (Biotage Selekt, 0-25% ethyl acetate/hexanes, 50 g Biotage SFar HC silica column) to give the title compound: ¹H NMR (500 MHz, CDCl₃) δ ppm 7.98 (s, 1H), 7.52 (dt, J=7.8, 1.1 Hz, 2H), 7.37 (dd, J=8.5, 7.5 Hz, 2H), 7.23-7.17 (m, 1H), 5.78 (d, J=48.5 Hz, 1H), 4.43 (dd, J=13.0, 0.7 Hz, 1H), 4.24 (dd, J=12.9, 2.7 Hz, 1H); MS (APCI) m/z=274 (M+H)⁺.

### 4-(Bromomethyl)-3-Fluoroquinolin-2(1H)-One

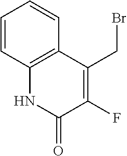 [SMILES: O=c1[nH]c2ccccc2c(CBr)c1F]

A mixture of 4-bromo-2-fluoro-3-oxo-N-phenylbutanamide (0.756 g, 2.76 mmol) in sulfuric acid (conc.) (2.90 mL) was heated to 45° C. for 1 hour. The reaction was then cooled to room temperature and poured over ice water. The resulting material was filtered, washed with water, 10% aqueous NaHCO₃, and again with water to give the title compound: ¹H NMR (600 MHz, CDCl₃) δ ppm 9.83 (s, 1H), 7.71 (dd, J=5.7, 3.3 Hz, 1H), 7.53 (dd, J=5.7, 3.3 Hz, 1H), 7.49 (d, J=8.9 Hz, 1H), 7.43 (d, J=8.9 Hz, 1H), 4.70 (d, J=2.1 Hz, 2H); MS (LSI) m/z=256 (M+H)⁺.

### 3-Fluoro-4-(Hydroxymethyl)Quinolin-2(1H)-One

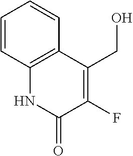 [SMILES: O=c1[nH]c2ccccc2c(CO)c1F]

A mixture of 4-(bromomethyl)-3-fluoroquinolin-2(1H)-one (0.529 g, 2.064 mmol) in 1 M aq. NaOH (4.13 mL, 4.13 mmol) was heated to 70° C. for 30 minutes. The reaction was cooled to room temperature and diluted with water. The aqueous layer was washed first with tert-butyl methyl ether and then with ethyl acetate. The aqueous layer was then acidified with 3M aqueous HCl to pH −2 and extracted with ethyl acetate 3×. The three latter ethyl acetate layers were combined, washed with brine, and dried over anhydrous sodium sulfate, filtered, and the filtrate was concentrated under reduced pressure to provide the title compound: ¹H NMR (400 MHz, CDCl₃) δ ppm 10.99 (s, 1H), 7.96 (tt, J=16.0, 8.0 Hz, 1H), 7.52-7.42 (m, 1H), 7.33 (q, J=9.4, 8.7 Hz, 2H), 5.16-4.95 (m, 2H); MS (LSI) m/z=194 (M+H)⁺.

### 3-Fluoro-2-Oxo-1,2-Dihydroquinoline-4-Carbaldehyde

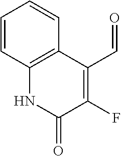 [SMILES: O=Cc1c(F)c(=O)[nH]c2ccccc12]

To a solution of 3-fluoro-4-(hydroxymethyl)quinolin-2(1H)-one (0.146 g, 0.755 mmol) in dichloromethane (5.03 mL) at 0° C. was added Dess-Martin Periodinane (1,1,1-tris(acetyloxy)-1,1-dihydro-1,2-benzodioxol-3-(1H)-one) (0.352 g, 0.830 mmol). The reaction was allowed to slowly warm to room temperature while stirring for 2 hours. Water (5 drops) was added, and the mixture was allowed to stir for 1 hour. The reaction was then diluted with ethyl acetate and washed twice with a 1:1 mixture of saturated sodium bicarbonate and saturated sodium thiosulfate. The organic phase was then washed with brine, dried over anhydrous sodium sulfate, filtered, and the filtrate was concentrated under reduced pressure to give the title compound: ¹H NMR (600 MHz, DMSO-d₆) δ ppm 12.69 (s, 1H), 10.56 (s, 1H), 8.49 (dt, J=8.3, 0.9 Hz, 1H), 7.56 (ddd, J=8.5, 7.2, 1.4 Hz, 1H), 7.40 (ddd, J=8.2, 1.3, 0.5 Hz, 1H), 7.32 (ddd, J=8.3, 7.2, 1.3 Hz, 1H); MS (LSI) m/z=192 (M+H)⁺.

### 3-Fluoro-2-Oxo-1,2-Dihydroquinoline-4-Carboxylic Acid

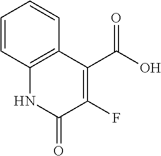 [SMILES: O=C(O)c1c(F)c(=O)[nH]c2ccccc12]

To a solution of 3-fluoro-2-oxo-1,2-dihydroquinoline-4-carbaldehyde (0.108 g, 0.564 mmol) and 2-methyl-2-butene (0.598 mL, 5.64 mmol) in a 4:1 mixture of tetrahydrofuran (5.00 mL) and tert-butanol (1.25 mL) was added a solution of sodium chlorite (0.204 g, 2.258 mmol) and sodium phosphate monobasic (0.542 g, 4.52 mmol) in water (5.00 mL) and the reaction stirred at room temperature for 1 hour. The reaction was then diluted with water and ethyl acetate and the layers separated with the desired product in the aqueous layer. The aqueous layer was acidified to pH −2 with concentrated HCl and extracted with ethyl acetate 3×. The combined organic layers were dried over anhydrous sodium sulfate, filtered, and the filtrate was concentrated under reduced pressure to give the title compound: ¹H NMR (500 MHz, DMSO-d 6) δ ppm 12.53 (s, 1H), 7.60 (dd, J=8.1, 1.4 Hz, 1H), 7.57 (ddd, J=8.5, 7.1, 1.4 Hz, 1H), 7.40 (dd, J=8.3, 1.2 Hz, 1H), 7.30 (td, J=7.6, 7.1, 1.1 Hz, 1H); MS (LSI) m/z=208 (M+H)⁺.

### 3,7,8-Trifluoro-2-Oxo-1,2-Dihydroquinoline-4-Carboxylic Acid

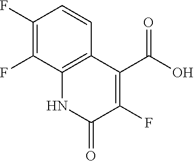 [SMILES: O=C(O)c1c(F)c(=O)[nH]c2c(F)c(F)ccc12]

### 3,7,8-Trifluoro-4-(Hydroxymethyl)Quinolin-2(1H)-One

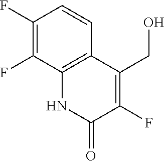 [SMILES: O=c1[nH]c2c(F)c(F)ccc2c(CO)c1F]

Synthesized following the procedure for 3-fluoro-4-(hydroxymethyl)quinolin-2(1H)-one, substituting 4-(bromomethyl)-3,7,8-trifluoroquinolin-2(1H)-one for 4-(bromomethyl)-3-fluoroquinolin-2(1H)-one: ¹H NMR (400 MHz, CDCl₃) δ ppm 10.33-9.71 (m, 1H), 7.74 (ddd, J=9.2, 4.8, 2.1 Hz, 1H), 7.15 (td, J=9.4, 7.5 Hz, 1H), 7.01-6.94 (m, 1H), 5.01 (d, J=2.6 Hz, 2H); MS (LSI) m/z=230 (M+H)⁺.

### 3,7,8-Trifluoro-2-Oxo-1,2-Dihydroquinoline-4-Carbaldehyde

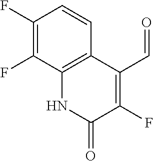 [SMILES: O=Cc1c(F)c(=O)[nH]c2c(F)c(F)ccc12]

Synthesized following procedure for 3-fluoro-2-oxo-1,2-dihydroquinoline-4-carbaldehyde, substituting 3,7,8-trifluoro-4-(hydroxymethyl)quinolin-2(1H)-one for 3-fluoro-4-(hydroxymethyl)quinolin-2(1H)-one: ¹H NMR (400 MHz, DMSO-d₆) δ ppm 12.70 (s, 1H), 10.51 (s, 1H), 8.33 (ddd, J=9.4, 5.4, 2.2 Hz, 1H), 7.36 (q, J=9.0 Hz, 1H); MS (LSI) m/z=260 (M+CH₃OH+H)³⁰.

### 3,7,8-Trifluoro-2-Oxo-1,2-Dihydroquinoline-4-Carboxylic Acid

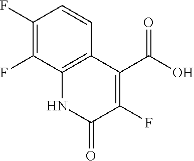 [SMILES: O=C(O)c1c(F)c(=O)[nH]c2c(F)c(F)ccc12]

Synthesized following procedure for 3-fluoro-2-oxo-1,2-dihydroquinoline-4-carboxylic acid, substituting 3,7,8-trifluoro-2-oxo-1,2-dihydroquinoline-4-carbaldehyde for 3-fluoro-2-oxo-1,2-dihydroquinoline-4-carbaldehyde: ¹H NMR (400 MHz, DMSO-d₆) δ ppm 12.76 (s, 1H), 7.47 (ddd, J=9.2, 5.1, 1.9 Hz, 1H), 7.37 (td, J=9.7, 7.2 Hz, 1H); MS (LSI) m/z=242 (M−H)⁺.

### 5-Methoxy-2-Oxo-1,2-Dihydroquinoline-4-Carboxylic Acid

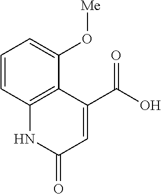 [SMILES: COc1cccc2[nH]c(=O)cc(C(=O)O)c12]

### 1-Acetyl-4-Methoxyindoline-2,3-Dione

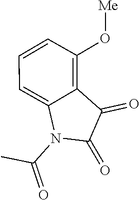 [SMILES: COc1cccc2c1C(=O)C(=O)N2C(C)=O]

A mixture of 4-methoxyindoline-2,3-dione (1.002 g, 5.66 mmol) in acetic anhydride (1.39 mL, 14.71 mmol) was heated to 155° C. After refluxing for 3 hours, the reaction was cooled to room temperature and the precipitated material was collected by filtration, washed with diethyl ether, and dried under reduced pressure to give the title compound: ¹H NMR (400 MHz, CDCl₃) δ ppm 7.98 (dd, J=8.2, 0.6 Hz, 1H), 7.65 (dd, J=8.7, 8.2 Hz, 1H), 6.82 (d, J=8.5 Hz, 1H), 4.02 (s, 3H), 2.73 (s, 3H); MS (APCI) m/z=252 (M+H)+.

### 5-Methoxy-2-Oxo-1,2-Dihydroquinoline-4-Carboxylic Acid

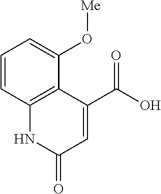 [SMILES: COc1cccc2[nH]c(=O)cc(C(=O)O)c12]

1-Acetyl-4-methoxyindoline-2,3-dione (1.159 g, 5.29 mmol) was added to a solution of sodium hydroxide (0.423 g, 10.57 mmol) in water (6.61 mL) and the resulting mixture heated to reflux for 3 hours. The reaction was cooled to 0° C. and to pH 1 with 3M aqueous HCl. The precipitated material was collected by filtration, washed with diethyl ether, and dried under reduced pressure to give the title compound: ¹H NMR (600 MHz, DMSO-d₆) δ ppm 13.27 (s, 1H), 11.90 (s, 1H), 7.49 (t, J=8.2 Hz, 1H), 6.95 (dd, J=8.3, 0.9 Hz, 1H), 6.78 (dd, J=8.3, 0.9 Hz, 1H), 6.31 (s, 1H), 3.82 (s, 3H); MS (APCI) m/z=220 (M+H)⁺.

### 8-Chloro-2-Oxo-1,2-Dihydroquinoline-4-Carboxylic Acid

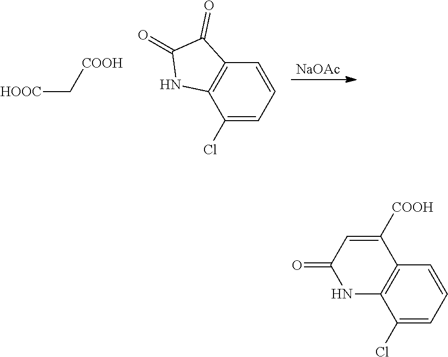 [SMILES: O=C(O)CC(=O)O ; O=C1Nc2c(Cl)cccc2C1=O ; O=C(O)c1cc(=O)[nH]c2c(Cl)cccc12 ; "NaOAc"]

A suspension of 7-chloroindoline-2,3-dione (970 mg, 5.34 mmol), malonic acid (667 mg, 6.41 mmol) and sodium acetate (1.10 g, 13.4 mmol) in acetic acid (7 mL) was heated at 100° C. overnight. The reaction mixture was brought to room temperature and added to water (35 mL) at 3° C. to precipitate the product, which was collected by filtration, washed with water and dried under vacuum to give the title compound. ¹H NMR (400 MHz, DMSO-d₆) δ ppm 10.91 (bs, 1H), 8.10 (dd, J=8.2, 1.3 Hz, 1H), 7.65 (dd, J=7.8, 1.3 Hz, 1H), 7.19 (dd, J=8.2, 7.8 Hz, 1H), 6.64 (s, 1H).

### 5-Fluoro-6-Methyl-2-Oxo-1,2-Dihydropyridine-4-Carboxylic Acid

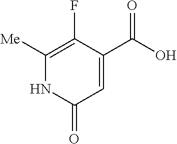 [SMILES: Cc1[nH]c(=O)cc(C(=O)O)c1F]

### 6-Bromo-3-Fluoro-2-Methylisonicotinic Acid

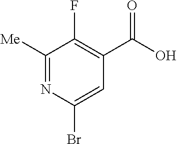 [SMILES: Cc1nc(Br)cc(C(=O)O)c1F]

A lithium diisopropylamide solution (2M in tetrahydrofuran/heptane/ethylbenzene, 6.00 mL, 12.00 mmol, Aldrich) was added dropwise to a solution of 6-bromo-3-fluoro-2-methylpyridine (1.90 g, 10 mmol, Combi-Blocks) in tetrahydrofuran (40 mL) at −78° C. After stirring for 1 hour, carbon dioxide was bubbled through the solution for 30 minutes. The reaction was then quenched with water and washed with ethyl acetate. The organic phase was then extracted twice with 1M NaOH (aq). The combined aqueous phases were then acidified with concentrated hydrochloric acid to pH 1 and the resulting mixture was extracted three times with ethyl acetate. The combined organic layers were then dried with magnesium sulfate, filtered, and the filtrate was concentrated under reduced pressure to give the title compound. ¹H NMR (600 MHz, DMSO-d₆) δ ppm 14.17 (s, 1H), 7.74 (dd, J=4.5, 0.8 Hz, 1H), 2.48 (dd, J=3.3, 0.6 Hz, 3H); MS (ESI+) m/z 234/236 (M+H)⁺.

### 5-Fluoro-6-Methyl-2-Oxo-1,2-Dihydropyridine-4-Carboxylic Acid

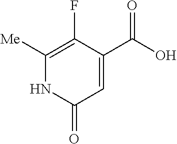 [SMILES: Cc1[nH]c(=O)cc(C(=O)O)c1F]

A mixture of 6-bromo-3-fluoro-2-methylisonicotinic acid (100 mg, 0.427 mmol), potassium hydroxide (144 mg, 2.56 mmol), copper(I) iodide (8.1 mg, 0.043 mmol), and 1,10-phenanthroline (15.4 mg, 0.085 mmol) in water (0.4 mL) and dimethyl sulfoxide (0.4 mL) was heated to 100° C. After 18 hours, the reaction was diluted with water and filtered through a pad of diatomaceous earth. The filtrate was then acidified with 1M HCl (aq) and concentrated under reduced pressure. The residue was purified by reverse phase HPLC (Waters Xbridge Prep C18 column, 80 mL/minute, 5-95% MeCN/0.1% TFA in water) and the product containing fractions were lyophilized to give the title compound. ¹H NMR (600 MHz, DMSO-d₆) δ ppm 12.28 (s, 1H), 6.58 (d, J=4.7 Hz, 1H), 2.54 (s, 1H), 2.26 (d, J=3.1 Hz, 3H); MS (ESI+) m/z 172 (M+H)⁺.

### 3-Fluoro-2-Methoxy-6-Methylisonicotinic Acid

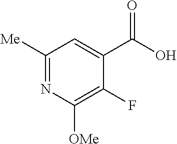 [SMILES: COc1nc(C)cc(C(=O)O)c1F]

A lithium diisopropylamide solution (2M in tetrahydrofuran/heptane/ethylbenzene, 1.28 mL, 2.55 mmol, Aldrich) was added dropwise to a solution of 3-fluoro-2-methoxy-6-methylpyridine (300 mg, 2.13 mmol, Combi-Blocks) in tetrahydrofuran (10.6 mL) at −78° C. After stirring for 1 hour, carbon dioxide was bubbled through the solution for 45 minutes. The reaction was then quenched with 1M NaOH (aq) and washed with ethyl acetate. The organic phase was then extracted twice with 1M NaOH (aq). The combined aqueous phases were then acidified with 1M HCl (aq) to pH 3 and the resulting mixture was extracted three times with dichloromethane. The combined organic layers were then dried with magnesium sulfate, filtered, and the filtrate was concentrated under reduced pressure to give the title compound ¹H NMR (500 MHz, DMSO-d₆) δ ppm 13.81 (s, 1H), 7.12 (dd, J=4.0, 0.8 Hz, 1H), 3.94 (s, 3H), 2.44-2.34 (m, 3H); MS (ESI+) m/z 186 (M+H)⁺.

### 3-Chloro-5-Fluoro-6-Methoxy-2-Methylisonicotinic Acid

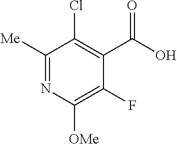 [SMILES: COc1nc(C)c(Cl)c(C(=O)O)c1F]

A solution of 3-fluoro-2-methoxy-6-methylisonicotinic acid (50 g, 270 mmol) in acetic acid (250 ml) was cooled to 20° C. N-Chlorosuccinimide (43.3 g, 324 mmol) was added, and the reaction mixture was stirred at 100° C. for 16 hours. TLC showed the starting material was consumed and a new spot discovered. The reaction mixture was poured into water and the cloudy solution was extracted with ethyl acetate (3×20 mL). The combined organic layers were dried with magnesium sulfate, filtered and the filtrate was concentrated under reduced pressure to give the title compound. ¹H NMR (600 MHz, DMSO-d₆) d 8.65 (s, 1H), 3.96 (s, 3H), 2.46 (d, J=1.3 Hz, 3H).

Compounds 4-85 were prepared by the conditions described in Examples 1˜4 substituting building blocks as needed.

### Biological Assays and Data

### SARS-CoV-2, SARS-CoV-1, MERS, 0C₄₃ and 229E Mpro Biochemical Protease Activity Assay

SARS-CoV-2 Mpro biochemical assays were performed in 8 μL in 384-well Proxi-Plus plate (PerkinElmer, Waltham, MA USA) at ambient temperature in the optimized assay buffer (50 mM Hepes, pH 7.1, 800 mM sodium citrate, 1 mM ethylenediaminetetraacetic acid, 1 mM TCEP ((tris(2-carboxyethyl)phosphine)), 0.005% BSA (bovine serum albumin)) with 1 nM of recombinant Mpro and 5 μM substrate. After 10 minutes of preincubation of inhibitor with Mpro in assay buffer, reactions were initiated by the addition of a FRET-compatible peptide substrate (TAMRA)-dPEG2-S-A-V-L-Q-S-G-dPEG2-K(QSY7)-amide. Fluorescence was measured at 20 minutes using a 531/579 excitation/emission filters on EnVision® (PerkinElmer, Waltham, MA USA). Data were normalized to DMSO minus no enzyme controls. IC₅₀ and Ki of inhibitor was analyzed using Dotmatics software on 11 points of inhibitor with varies concentration. Each inhibitor was tested at least twice at different dates. 2 nM Mpro and 5 μM substrate for SARS-CoV-1, 5 nM Mpro and 5 μM for MERS, 1 nM of Mpro and 2 μM substrate for 0C43, 3 nM Mpro and 2 μM substrate for 229E were used in respective Mpro biochemical protease activity assays.

### SARS-CoV-2/HeLa-ACE2 High-Content Screening Assay

Compounds were acoustically transferred into 384-well μclear-bottom plates (Greiner) and HeLa-ACE2 cells were seeded in the plates in 2% FBS (fetal bovine serum) at a density of 1.0×10³ cells per well. Plated cells were transported to the BSL3 facility where SARS-CoV-2 (strain USA-WA1/2020 propagated in Vero E6 cells) diluted in assay media was added to achieve −30-50% infected cells. Plates were incubated for 24 hours at 34° C. 5% CO₂, and then fixed with 8% formaldehyde. Fixed cells were stained with human polyclonal sera as the primary antibody, goat anti-human H+L conjugated Alexa Fluor™ Plus 488 (Thermo Fisher Scientific) as the secondary antibody, and antifade-46-diamidino-2-phenylindole (DAPI; Thermo Fisher Scientific) to stain DNA, with PBS 0.05% Tween 20 (phosphate buffered saline) washes in between fixation and subsequent primary and secondary antibody staining.

Plates were imaged using the ImageXpress Micro Confocal High-Content Imaging System (Molecular Devices) with a 10× objective, with 4 fields imaged per well. Images were analyzed using the MetaXpress® Multi-Wavelength Cell Scoring Application Module (MetaXpress, Sunnyvale, CA USA), with DAPI staining identifying the host-cell nuclei (the total number of cells in the images) and the SARS-CoV-2 immunofluorescence signal leading to identification of infected cells.

### Uninfected Host Cell Cytotoxicity Counter Screen

Compounds were acoustically transferred into 1,536-well plates (Corning, Glendale, AZ USA). HeLa-ACE2 cells were maintained as described for the infection assay and seeded in the assay-ready plates at 400 cells/well in DMEM (Dulbecco's Modified Eagle Medium, Thermofisher Scientific) with 2% FBS. Plates were incubated for 24 hours at 37° C. 5% CO₂. To assess cell viability, 2 μL of 50% Cell-Titer Glo (Promega™ Madison, WI USA) diluted in water was added to the cells and luminescence was measured on an EnVision Plate Reader (Perkin Elmer).

### Data Analysis

Primary in vitro screen and the host cell cytotoxicity counter screen data were uploaded to Genedata Screener®, Version 16.0. Data were normalized to neutral (DMSO) minus inhibitor controls (2.5 μM remdesivir for antiviral effect and 10 μM puromycin for infected host cell toxicity). For the uninfected host cell cytotoxicity counter screen 40 μM puromycin (MilliporeSigma) was used as the positive control. For dose response experiments compounds were tested in technical triplicates on different assay plates and dose curves were fitted with the four parameter Hill Equation.

### OC43 Antiviral and Cellular Toxicity Assays.

Huh-7 cells were seeded (10,000 cells per well) in 96-well plates. The following day, the culture media were replaced with fresh media containing 2% FBS and serially diluted compounds, in the presence or absence of an inoculum containing approximately 100 focus forming units of 0C₄₃ virus. The plates were incubated at 33° C., 5% CO₂ for 48 hours. Plates with OC43 infection were fixed and immunostained for viral nucleoprotein to identify the infected cells, while plates with uninfected cells were evaluated for cell viability (i.e. cellular toxicity of compounds) by CellTiter Glo according to manufacturer's instructions. For immunostaining, infected cells were fixed in 4% formalin solution with nuclear stain Hoechst 33341 for 20 minutes, blocked with 10% BSA plus 0.5% Triton X-100 for 30 minutes, stained with anti-OC43 antibody (MilliporeSigma) diluted 1:2,000 for 1 hour, washed with PBS three times, stained with anti-mouse-AlexaFluor488 antibody diluted 1:1,000 for 1 hour, and washed with PBS. The number of 0C₄₃ foci in each well was quantitated by high-content microscopy using the Thermo CellInsight™ CX7 LZR platform. The EC₅₀ and CC₅₀ values were calculated by non-linear regression analysis using the GraphPad Prism (version 5) software.

### 229E Antiviral and Cellular Toxicity Assays.

Huh-7 cells were seeded (5,000 cells per well) in 96-well plates. The following day, the culture media were replaced with fresh media containing 2% FBS and serially diluted compounds, in the presence or absence of a 229E inoculum (MOI of −0.03). The plates were incubated at 35° C., 5% CO₂ for 5 days to allow the virus to induce extensive cytopathic effects in infected cells. The viability of the cells incubated with compounds in the presence or absence of 229E virus was determined by CellTiter-Glo® (Promega® Madison, WI USA) according to manufacturer's instructions. The EC₅₀ and CC₅₀ values were calculated by the GraphPad Prism (version 5) software.

Tables 1, 2, and 3 are lists of exemplary compounds and that were tested according to the protocols described herein and their respective spectral and inhibition data.

TABLE 1
Compound Examples and Spectral Data

| Example | Structure | NMR Data | MS Data |
|---|---|---|---|
| Compound 1 | 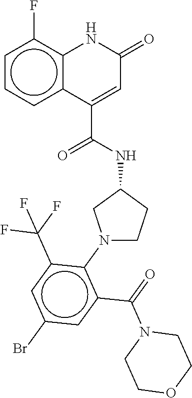 | ¹H NMR (400 MHz, DMSO- d₆) δ ppm 11.95 (s, 1H), 8.98- 8.78 (m, 1H), 7.91-7.87 (m, 1H), 7.86-7.82 (m, 1H), 7.56-7.50 (m, 1H), 7.50- 7.43 (m, 1H), 7.22-7.15 (m, 1H), 6.66-6.59 (m, 1H), 4.48-4.37 (m, 1H), 3.74- 3.42 (m, 7H), 3.32-3.06 (m, 5H), 2.28-2.14 (m, 1H), 2.01-1.85 (m, 1H); ¹H NMR (400 MHz, DMSO- d₆, 120° C.) δ ppm 11.24 (bs, 1H), 8.37 (s, 1H), 7.79 (d, J = 2.4 Hz, 1H), 7.67 (d, J = 2.4 Hz, 1H), 7.59-7.52 (m, 1H), 7.39-7.31 (m, 1H), 7.17- 7.10 (m, 1H), 6.59 (s, 1H), 4.52-4.41 (m, 1H), 3.70- 3.20 (m, 11H), 3.19-3.12 (m, 1H), 2.28-2.18 (m, 1H), 2.01-1.91 (m, 1H); ¹³C NMR (101 MHz, DMSO- d₆, 90° C.) δ ppm 165.40, | MS (ESI+) m/z 611/613 (M + H)⁺ |
| | | 164.92, 160.40, 148.65 (d, J = | |
| | | 246.3 Hz), 145.32-145.24 | |
| | | (m), 143.44, 139.57, 134.05, | |
| | | 130.85 (q, J = 29.5 Hz), | |
| | | 130.08 (q, J = 5.8 Hz), 127.91- | |
| | | 127.69 (m), 122.58 (q, J = | |
| | | 274.1 Hz), 121.30-121.22 | |
| | | (m), 121.24-121.16 (m), | |
| | | 120.59, 118.10-118.02 (m), | |
| | | 116.72, 115.21 (d, J = 17.3 | |
| | | Hz), 65.15, 57.22, 50.36, | |
| | | 49.59, 46.82, 41.35, 30.91; | |
| | | ¹⁹F NMR (376 MHz, DMSO- | |
| | | d6, 90° C.) δ ppm −60.16, (s), | |
| | | −131.01 (dd, J = 11.0, 5.2 Hz). | |
| Compound 2 | 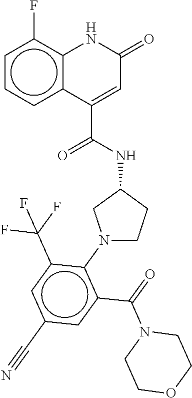 | ¹H NMR (500 MHz, DMSO- d₆) δ ppm 11.96 (s, 1H), 9.03- 8.84 (m, 1H), 8.21-8.18 (m, 1H), 7.98-7.93 (m, 1H), 7.56-7.50 (m, 1H), 7.49- 7.43 (m, 1H), 7.21-7.15 (m, 1H), 6.66-6.59 (m, 1H), 4.51-4.40 (m, 1H), 3.73- 3.55 (m, 6H), 3.54-3.22 (m, 6H), 2.29-2.18 (m, 1H), 2.07-1.95 (m, 1H). ¹H NMR (400 MHz, DMSO- d₆, 90° C.) δ ppm 11.47 (bs, 1H), 8.77-8.47 (m, 1H), 8.09 (d, J = 2.1 Hz, 1H), 7.88 (d, J = 2.1 Hz, 1H), 7.54 (dd, J = 8.1, 1.2 Hz, 1H), 7.39 (ddd, J = 11.0, 8.1, 1.2 Hz, 1H), 7.15 (ddd, J = 8.1, 8.1, 5.1 Hz, 1H), 6.60 (s, 1H), 4.54-4.43 (m, 1H), 3.74- 3.20 (m, 12H), 2.31-2.21 (m, 1H), 2.06-1.96 (m, 1H). ¹³C NMR (101 MHz, DMSO- | MS (APCI+) m/z 558 (M + H)⁺ |
| | | d6, 90° C.) δ ppm 166.61, | |
| | | 165.97, 161.31, 149.59 (d, J = | |
| | | 246.3 Hz), 148.95, 146.10- | |
| | | 146.03 (m), 136.00, 135.65, | |
| | | 133.13 (q, J = 6.1 Hz), 128.84- | |
| | | 128.63 (m), 123.76 (q, J = | |
| | | 2.73.7 Hz), 122.19, 122.16, | |
| | | 122.12, 121.60, 118.99- | |
| | | 118.91 (m), 117.75, 116.17 | |
| | | (d, J = 17.3 Hz), 105.57, | |
| | | 66.04, 57.95, 51.06, 50.35, | |
| | | 47.74, 42.35, 31.54. | |
| | | ¹⁹F NMR (376 MHz, DMSO- | |
| | | d₆) δ ppm −57.54-−58.10 | |
| | | (m), −130.02-−130.15 (m); | |
| | | 19F NMR (376 MHz, | |
| | | DMSO-d₆, 90° C.) δ ppm −58.37- | |
| | | −58.97 (m), −130.96 (dd, | |
| | | J = 11.05, 5.1 Hz). | |
| Compound 3 | 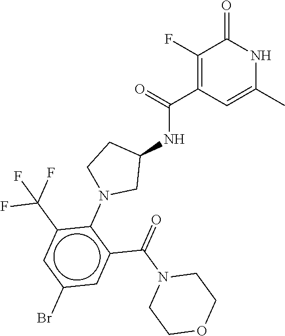 | ¹H NMR (400 MHz, DMSO- d₆) δ ppm 12.22 (s, 1H), 8.60 (dd, J = 55.9, 6.7 Hz, 1H), 7.88 (d, J = 2.4 Hz, 1H), 7.83 (dd, J = 5.3, 2.4 Hz, 1H), 5.97 (dd, J = 11.2, 4.6 Hz, 1H), 4.35 (dt, J = 15.5, 6.9 Hz, 1H), 3.77-3.37 (m, 7H), 3.28-3.10 (m, 4H), 3.02 (ddd, J = 18.3, 8.7, 6.5 Hz, 1H), 2.16 (s, 4H), 1.86 (ddq, J = 18.9, 14.5, 7.4 Hz, 1H) | MS (ESI+) m/z 575/577 (M + H)⁺ |
| Compound 4 | 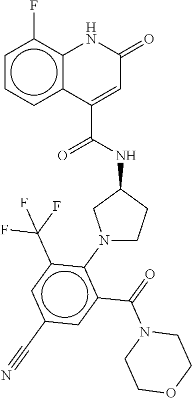 | ¹H NMR (600 MHz, DMSO- d₆) δ ppm 11.96 (s, 1H), 8.93 (dd, J = 94.5, 6.1 Hz, 1H), 8.19-8.17 (m, 1H), 7.95 (dd, J = 16.7, 2.1 Hz, 1H), 7.55- 7.43 (m, 2H), 7.22-7.14 (m, 1H), 6.62 (d, J = 26.8 Hz, 1H), 4.48-4.41 (m, 1H), 3.71-3.37 (m, 10H), 3.31-3.22 (m, 2H), 2.32-2.17 (m, 1H), 2.06-1.95 (m, 1H). | MS (APCI+) m/z 558 (M + H)⁺ |
| Compound 5 | 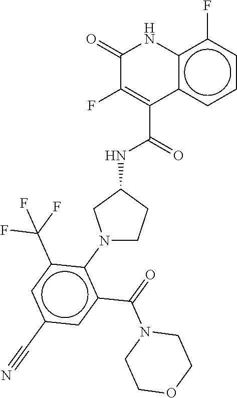 | ¹H NMR (400 MHz, DMSO- d₆, 90° C.) δ ppm 12.14 (s, 1H), 8.87-8.83 (m, 1H), 8.09 (d, J = 2.1 Hz, 1H), 7.88 (d, J = 2.1 Hz, 1H), 7.45- 7.35 (m, 1H), 7.33-7.27 (m, 1H), 7.22 (td, J = 8.1, 5.0 Hz, 1H), 4.53 (d, J = 7.2 Hz, 1H), 3.72-3.64 (m, 2H), 3.61 (s, 4H), 3.40 (s, 2H), 3.26 (s, 3H), 2.35-2.24 (m, 1H), 1.95 (dd, J = 12.9, 6.6 Hz, 1H). | MS (ESI+) m/z 576 (M + H)⁺ |
| Compound 6 | 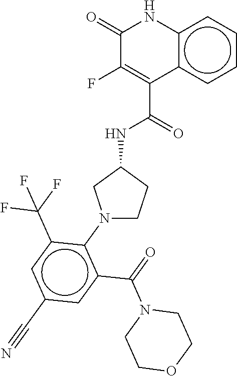 | ¹H NMR (40 0MHz, DMSO- d₆, 90° C.) δ ppm 12.13 (s, 1H), 8.82 (s, 1H), 8.09 (d, J = 2.1 Hz, 1H), 7.88 (d, J = 2.1 Hz, 1H), 7.55-7.43 (m, 2H), 7.42-7.35 (m, 1H), 7.23 (t, J = 7.4 Hz, 1H), 4.54 (d, J = 6.9 Hz, 1H), 3.73-3.64 (m, 1H), 3.62 (s, 3H), 3.41 (s, 3H), 3.26 (s, 4H), 2.28 (d, J = 6.7 Hz, 1H), 1.96 (dd, J = 12.8, 6.6 Hz, 1H), 1.30-1.23 (m, 1H). | MS (ESI+) m/z 558 (M + H)⁺ |
| Compound 7 | 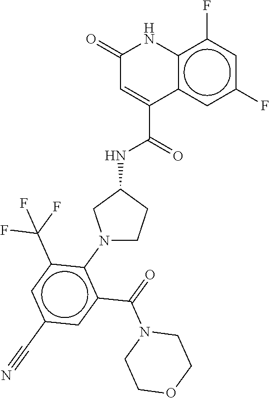 | ¹H NMR (600 MHz, DMSO- d₆) δ ppm 12.06 (s, 1H), 8.96 (dd, J = 93.2, 6.0 Hz, 1H), 8.19 (dd, J = 6.6, 2.1 Hz, 1H), 7.95 (dd, J = 17.7, 2.1 Hz, 1H), 7.62 (ddt, J = 11.2, 8.7, 2.6 Hz, 1H), 7.38 (tt, J = 10.1, 2.1 Hz, 1H), 6.75 (d, J = 31.0 Hz, 1H), 4.45 (dh, J = 24.1, 5.9 Hz, 1H), 3.76-3.37 (m, 11H), 3.29- 3.20 (m, 1H), 2.29-2.17 (m, 1H), 2.08-1.96 (m, 1H). | MS (APCI)+) m/z 576 (M + H)⁺ |
| Compound 8 | 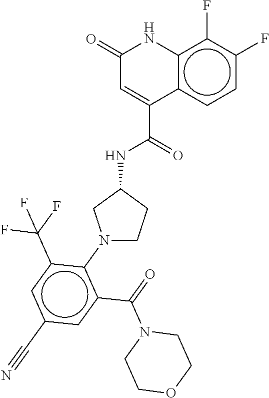 | ¹H NMR (400 MHz, DMSO- d₆) δ ppm 12.20 (s, 1H), 8.94 (dd, J = 61.9, 6.1 Hz, 1H), 8.19 (dd, J = 4.6, 2.1 Hz, 1H), 7.95 (dd, J = 12.7, 2.1 Hz, 1H), 7.63-7.52 (m, 1H), 7.32-7.20 (m, 1H), 6.61 (d, J = 19.8 Hz, 1H), 4.44 (dq, J = 2.6, 6.0 Hz, 1H), 3.79-3.37 (m, 11H), 3.24 (dd, J = 9.7, 4.5 Hz, 1H), 2.23 (tq, J = 13.3, 6.9 Hz, 1H), 2.01 (ddt, J = 19.1, 12.7, 6.2 Hz, 1H) | MS (APCI+) m/z 576 (M + H)⁺ |
| Compound 9 | 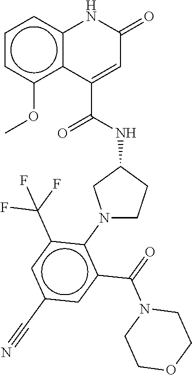 | ¹H NMR (400 MHz, DMSO- d₆, 90° C.) δ ppm 11.48 (s, 1H), 8.15-8.00 (m, 2H), 7.83 (d, J = 2.1 Hz, 1H), 7.41 (t, J = 8.2 Hz, 1H), 6.93 (d, J = 8.3 Hz, 1H), 6.71 (d, J = 8.1 Hz, 1H), 6.15 (s, 1H), 4.47-4.35 (m, 1H), 3.74 (s, 3H), 3.70- 3.47 (m, 7H), 3.45-3.12 (m, 5H), 2.23 (dt, J = 12.8, 6.7 Hz, 1H), 1.95 (dd, J = 12.2, 6.5 Hz, 1H). | MS (APCI+) m/z 570.32 (M + H)⁺ |
| Compound 10 | 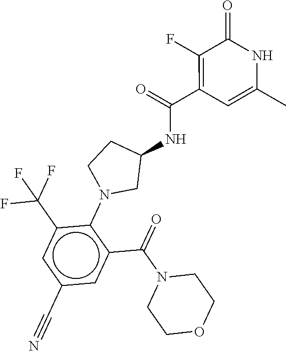 | ¹H NMR (500 MHz, DMSO- d₆) δ ppm 12.21 (s, 1H), 8.78 (d, J = 6.3 Hz, 0.5H), 8.63 (d, J = 6.5 Hz, 0.5H), 8.20 (dd, J = 5.8, 2.1 Hz, 1H), 7.96 (dd, J = 10.1, 2.1 Hz, 1H), 5.98 (dd, J = 14.1, 4.6 Hz, 1H), 4.39 (dh, J = 25.6, 6.3 Hz, 1H), 3.82-3.12 (m, 12H), 2.28- 2.14 (m, 4H), 1.94 (dddt, J = 14.3, 12.4, 7.7, 6.2 Hz, 1H). | MS (APCI⁺) m/z 522.23 (M + H)⁺ |
| Compound 11 | 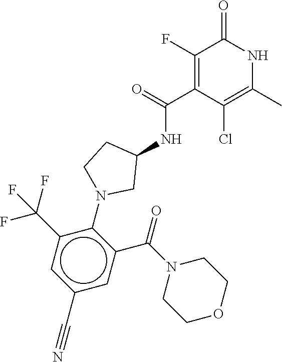 | ¹H NMR (500 MHz, DMSO- d₆) δ ppm 12.65 (s, 1H), 9.06 (d, J = 6.3 Hz, 0.5H), 8.93 (d, J = 6.6 Hz, 0.5H), 8.18 (dd, J = 6.3, 2.1 Hz, 1H), 7.94 (dd, J = 9.0, 2.1 Hz, 1H), 4.38 (dq, J = 29.1, 6.3 Hz, 1H), 3.74- 3.08 (m, 12H), 2.29-2.15 (m, 4H), 1.89 (tq, J = 13.5, 6.7 Hz, 1H). | MS (APCI+) m/z 556.30 (M + H)⁺ |
| Compound 12 | 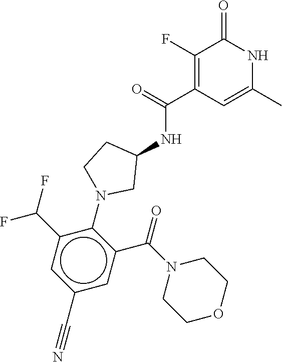 | ¹H NMR (500 MHz, DMSO- d₆) δ ppm 12.23 (s, 1H), 8.84 (d, J = 6.6 Hz, 0.5H), 8.74 (d, J = 6.7 Hz, 0.5H), 7.99 (d, J = 2.1 Hz, 0.5H), 7.98 (d, J = 2.1 Hz, 0.5H), 7.80 (d, J = 2.1 Hz, 0.5H), 7.78 (d, J = 2.1 Hz, 0.5H), 7.25 (td, J = 54.4, 5.8 Hz, 1H), 5.99-5.94 (m, 1H), 4.49-4.37 (m, 1H), 3.31 (s, 11.5H), 3.10-3.05 (m, 0.5H), 2.29-2.17 (m, 1H), 2.16 (s, 3H), 1.99-1.88 (m, 1H). | MS (ESI+) m/z 504.3 (M + H)⁺ |
| Compound 13 | 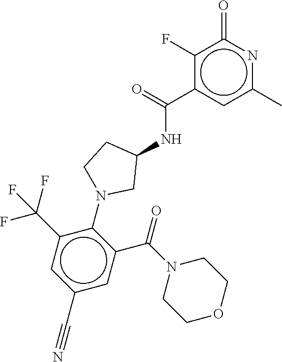 | ¹H NMR (500 MHz, DMSO- d₆) δ ppm 12.21 (s, 1H), 8.81- 8.19 (m, 2H), 8.03 (dd, J = 4 1.9, 2.1 Hz, 1H), 5.98 (dd, J = 27.4, 4.5 Hz, 1H), 4.60- 4.23 (m, 1H), 3.54-2.99 (m, 8H), 2.15 (t, J = 1.0 Hz, 4H), 1.86-1.70 (m, 1H), 0.96 (dd, J = 11.8, 6.0 Hz, 3H). | MS (APCI+) m/z 536 (M + H)⁺ |
| Compound 14 | 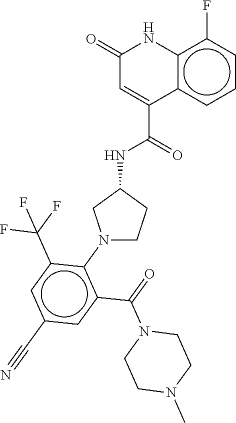 | 1H NMR (600 MHz, DMSO- d₆) δ ppm 11.96 (s, 1H), 8.92 (dd, J = 91.6, 6.2 Hz, 1H), 8.19 (dd, J = 7.1, 2.1 Hz, 1H), 7.91 (dd, J = 20.1, 2.1 Hz, 1H), 7.58-7.41 (m, 2H), 7.18 (tdd, J = 8.1, 5.0, 1.2 Hz, 1H), 6.62 (d, J = 22.7 Hz, 1H), 4.45 (dq, J = 23.2, 5.9 Hz, 1H), 3.63 (ddd, J = 16.0, 9.5, 6.3 Hz, 3H), 3.51-3.38 (m, 1H), 3.27-3.20 (m, 1H), 3.17 (d, J = 5.2 Hz, 1H), 2.37 (d, J = 53.9 Hz, 3H), 2.28- 2.08 (m, 5H), 2.05-1.94 (m, 1H), 1.32-1.16 (m, 2H). | MS (APCI+) m/z 571 (M + H)⁺ |
| Compound 15 | 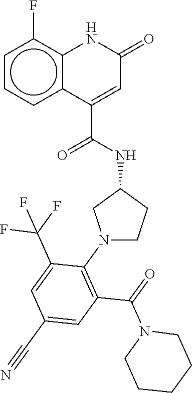 | ¹H NMR (400 MHz, DMSO- d₆, 90° C.) δ ppm 8.79-8.49 (m, 1H), 8.07 (d, J = 2.1 Hz, 1H), 7.81 (d, J = 2.1 Hz, 1H), 7.54 (d, J = 8.2 Hz, 1H), 7.39 (ddd, J = 11.0, 8.1, 1.2 Hz, 1H), 7.15 (td, J = 8.1, 5.1 Hz, 1H), 6.60 (s, 1H), 4.48 (s, 1H), 3.78-3.12 (m, 8H), 2.29-2.19 (m, 1H), 2.07- 1.94 (m, 1H), 1.67-1.35 (m, 6H). | MS (APCI+) m/z: 598.4 (M + H)⁺ |
| Compound 16 | 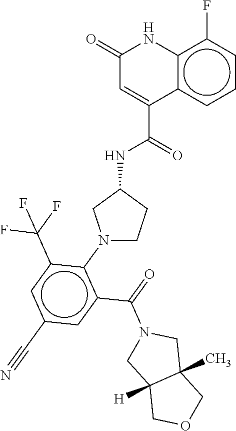 | ¹H NMR (400 MHz, DMSO- d₆, 90° C.) δ ppm 8.64-8.58 (m, 1H), 8.08 (d, J = 2.1 Hz, 1H), 7.95-7.91 (m, 1H), 7.54 (d, J = 8.2 Hz, 1H), 7.44- 7.34 (m, 1H), 7.20-7.10 (m, 1H), 6.60 (s, 1H), 4.55- 4.46 (m, 1H), 4.04-3.16 (m, 12H), 2.31-2.18 (m, 1H), 2.03-1.99 (m, 1H), 1.34- 1.06 (m, 4H). | MS (APCI+) m/z 570.4 (M + H)⁺ |
| Compound 17 | 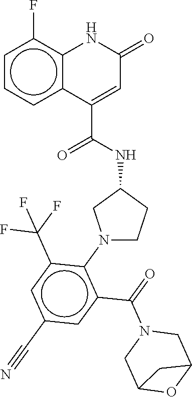 | ¹H NMR (400 MHz, DMSO- d₆, 90° C.) δ ppm 11.50 (s, 1H), 8.65-8.60 (m, 1H), 8.10 (d, J = 2.0 Hz, 1H), 8.02- 7.96 (m, 1H), 7.56-7.48 (m, 1H), 7.44-7.34 (m, 1H), 7.20-7.10 (m, 1H), 6.59 (s, 1H), 4.65-4.60 (m, 1H), 4.55-4.44 (m, 2H), 3.93- 3.82 (m, 1H), 3.72-3.56 (m, 3H), 3.52-3.34 (m, 3H), 3.34-3.24 (m, 1H), 3.15- 3.07 (m, 1H), 2.25 (dq, J = 13.7, 7.0 Hz, 1H), 2.05-1.77 (m, 2H). | MS (APCI+) m/z 584.4 (M + H)⁺ |
| Compound 18 | 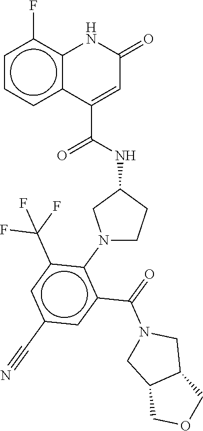 | ¹H NMR (400 MHz, DMSO- d₆, 90° C.) δ ppm 8.60 (d, J = 6.4 Hz, 1H), 8.08 (d, J = 2.1 Hz, 1H), 7.89 (d, J = 2.1 Hz, 1H), 7.54 (d, J = 8.2 Hz, 1H), 7.39 (ddd, J = 11.0, 8.1, 1.2 Hz, 1H), 7.20-7.10 (m, 1H), 6.60 (s, 1H), 4.54-4.45 (m, 1H), 3.90-3.31 (m, 9H), 3.31-3.23 (m, 1H), 3.23- 3.07 (m, 1H), 2.97-2.90 (m, 3H), 2.31-2.18 (m, 1H), 2.06-1.93 (m, 1H). | MS (APCI+) m/z 615.4 (M + H)⁺ |
| Compound 19 | 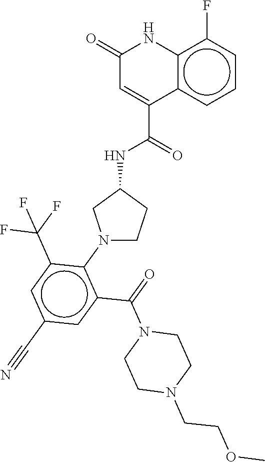 | ¹H NMR (400 MHz, DMSO- d₆, 90° C.) δ ppm 11.51 (s, 1H), 8.77-8.48 (m, 1H), 8.08 (d, J = 2.1 Hz, 1H), 7.82 (d, J = 2.1 Hz, 1H), 7.55 (d, J = 8.1 Hz, 1H), 7.44-7.34 (m, 1H), 7.23-7.10 (m, 1H), 6.60 (s, 1H), 4.48 (s, 1H), 3.74-3.53 (m, 3H), 3.50- 3.13 (m, 11H), 2.57-2.33 (m, 5H), 2.28-2.18 (m, 1H), 2.07-1.93 (m, 1H). | MS (APCI+) m/z 542.2 (M + H)⁺ |
| Compound 20 | 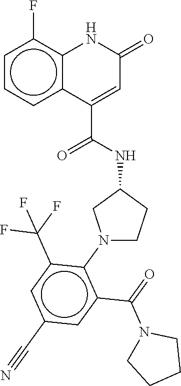 | ¹H NMR (400 MHz, DMSO- d₆, 90° C.) δ ppm 8.61 (d, J = 6.4 Hz, 1H), 8.07 (d, J = 2.1 Hz, 1H), 7.89 (d, J = 2.1 Hz, 1H), 7.53 (d, J = 8.2 Hz, 1H), 7.39 (ddd, J = 11.0, 8.0, 1.2 Hz, 1H), 7.15 (td, J = 8.1, 5.1 Hz, 1H), 6.60 (s, 1H), 4.48 (q, J = 6.0 Hz, 1H), 3.63 (dd, J = 9.6, 6.4 Hz, 1H), 3.51-3.16 (m, 7H), 2.31-2.18 (m, 1H), 2.06-1.94 (m, 1H), 1.86 (t, J = 8.2 Hz, 4H). | MS (APCI+) m/z 578.4 (M + H)⁺ |
| Compound 21 | 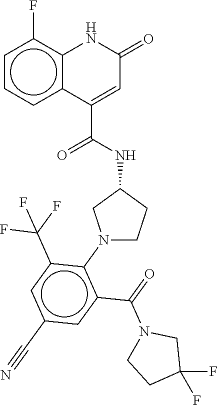 | ¹H NMR (400 MHz, DMSO- d₆, 90° C.) δ ppm 8.62 (d, J = 6.4 Hz, 1H), 8.11 (d, J = 2.1 Hz, 1H), 7.98-7.91 (m, 1H), 7.53 (d, J = 8.2 Hz, 1H), 7.44- 7.34 (m, 1H), 7.15 (td, J = 8.1, 5.1 Hz, 1H), 6.60 (s, 1H), 4.49 (q, J = 6.3 Hz, 1H), 3.96- 3.49 (m, 5H), 3.49-3.16 (m, 3H), 2.48-2.39 (m, 2H), 2.31-2.19 (m, 1H), 2.06- 1.94 (m, 1H). | MS (APCI+) m/z 564.3 (M + H)⁺ |
| Compound 22 | 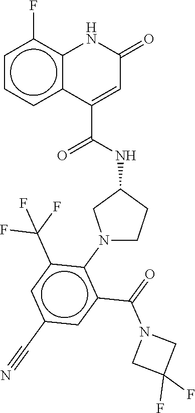 | ¹H NMR (400 MHz, DMSO- d₆, 90° C.) δ ppm 8.64 (d, J = 6.3 Hz, 1H), 8.13 (d, J = 2.1 Hz, 1H), 8.04 (d, J = 2.1 Hz, 1H), 7.53 (d, J = 8.2 Hz, 1H), 7.44-7.34 (m, 1H), 7.15 (td, J = 8.1, 5.1 Hz, 1H), 6.60 (s, 1H), 4.51 (td, J = 12.2, 4.6 Hz, 5H), 3.66 (dd, J = 9.7, 6.5 Hz, 1H), 3.50-3.36 (m, 2H), 3.30 (dd, J = 9.7, 5.3 Hz, 1H), 2.31-2.23 (m, 1H), 2.03 (dt, J = 12.6, 6.2 Hz, 1H). | MS (APCI+) m/z 613.4 (M + H)⁺ |
| Compound 23 | 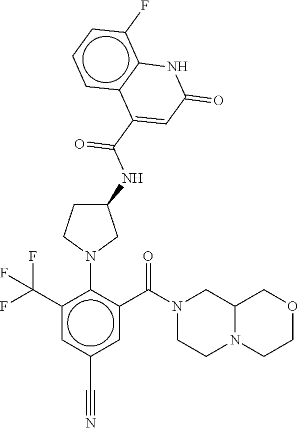 | ¹H NMR (400 MHz, DMSO- d₆, 90° C.) δ ppm 11.51 (s, 1H), 8.82-8.48 (m, 1H), 8.16-8.04 (m, 1H), 7.90- 7.74 (m, 1H), 7.61-7.49 (m, 1H), 7.39 (ddd, J = 11.0, 8.1, 1.2 Hz, 1H), 7.15 (td, J = 8.1, 5.1 Hz, 1H), 6.61 (s, 1H), 4.60-4.16 (m, 2H), 3.79- 3.09 (m, 11H), 2.85-2.55 (m, 2H), 2.13 (d, J = 99.5 Hz, 5H). | MS (APCI+) m/z 641.4 (M + H)⁺ |
| Compound 24 | 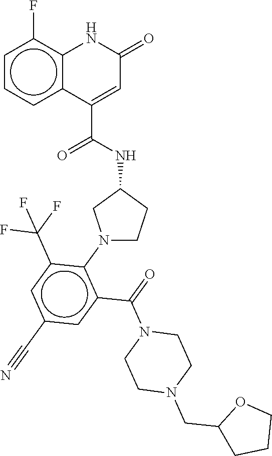 | ¹H NMR (400 MHz, DMSO- d₆, 90° C.) δ ppm 8.72-8.46 (m, 1H), 8.08 (d, J = 2.1 Hz, 1H), 7.82 (d, J = 2.1 Hz, 1H), 7.55 (d, J = 8.2 Hz, 1H), 7.39 (ddd, J = 11.0, 8.1, 1.2 Hz, 1H), 7.20-7.10 (m, 1H), 6.60 (s, 1H), 4.57-4.38 (m, 1H), 3.97-3.87 (m, 1H), 3.77-3.54 (m, 4H), 3.49- 3.16 (m, 4H), 2.55 (s, 5H), 2.45-2.34 (m, 3H), 2.25 (dd, J = 12.7, 6.7 Hz, 1H), 2.04- 1.97 (m, 1H), 1.95-1.70 (m, 3H), 1.58-1.38 (m, 1H). | MS (APCI+) m/z 649.3 (M + H)⁺ |
| Compound 25 | 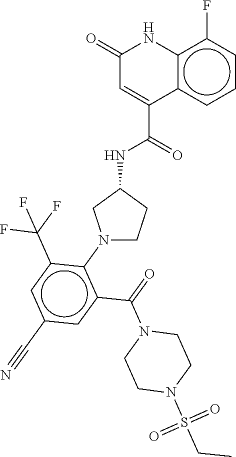 | ¹H NMR (400 MHz, DMSO- d₆, 90° C.) δ ppm 8.60 (s, 1H), 8.11 (d, J = 2.1 Hz, 1H), 7.92 (d, J = 2.1 Hz, 1H), 7.54 (d, J = 8.1 Hz, 1H), 7.39 (ddd, J = 11.0, 8.1, 1.2 Hz, 1H), 7.15 (td, J = 8.1, 5.1 Hz, 1H), 6.61 (s, 1H), 4.54-4.38 (m, 1H), 3.29 (d, J = 31.0 Hz, 11H), 3.12-3.04 (m, 3H), 2.26 (dd, J = 12.5, 6.5 Hz, 1H), 2.02 (s, 1H), 1.28-1.17 (m, 3H). | MS (APCI+) mm/z/z 581.3 (M + H)⁺ |
| Compound 26 | 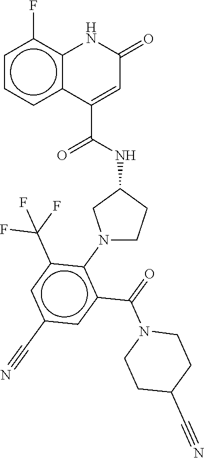 | ¹H NMR (400 MHz, DMSO- d₆, 90° C.) δ ppm 11.69- 11.24 (m, 1H), 8.76-8.47 (m, 1H), 8.09 (d, J = 2.1 Hz, 1H), 8.02-7.84 (m, 1H), 7.54 (d, J = 8.2 Hz, 1H), 7.39 (ddd, J = 11.0, 8.1, 1.2 Hz, 1H), 7.15 (td, J = 8.1, 5.1 Hz, 1H), 6.60 (s, 1H), 4.53-4.41 (m, 1H), 4.14-3.55 (m, 2H), 3.50-3.09 (m, 6H), 2.27- 2.22 (m, 1H), 1.98 (s, 6H). | MS (APCI+) m/z 571.3 (M + H)⁺ |
| Compound 27 | 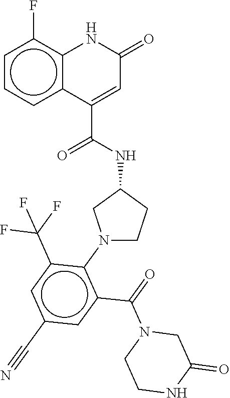 | ¹H NMR (400 MHz, DMSO- d₆, 90° C.) δ ppm 8.62 (s, 1H), 8.11 (d, J = 2.1 Hz, 1H), 7.94-7.90 (m, 1H), 7.86- 7.82 (m, 1H), 7.53 (d, J = 8.3 Hz, 1H), 7.44-7.34 (m, 1H), 7.20-7.11 (m, 1H), 6.60 (s, 1H), 4.50-4.44 (m, 1H), 4.17-4.00 (m, 1H), 3.92- 3.71 (m, 1H), 3.62 (dd, J = 9.5, 6.5 Hz, 1H), 3.54-3.14 (m, 6H), 2.29-2.19 (m, 1H), 2.04-1.93 (m, 1H). | MS (APCI+) m/z 571.3 (M + H)⁺ |
| Compound 28 | 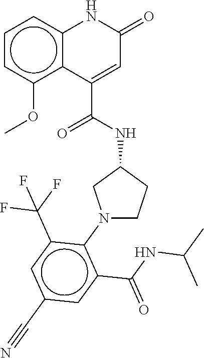 | ¹H NMR (600 MHz, DMSO- d₆) δ ppm 11.82 (s, 1H), 8.52 (d, J = 7.5 Hz, 1H), 8.33 (d, J = 7.0 Hz, 1H), 7.89 (d, J = 2.5 Hz, 1H), 7.71 (d, J = 2.4 Hz, 1H), 7.46 (t, J = 8.2 Hz, 1H), 6.94 (dd, J = 8.3, 0.9 Hz, 1H), 6.74 (dd, J = 8.3, 0.9 Hz, 1H), 6.15 (d, J = 1.9 Hz, 1H), 4.45 (h, J = 6.9 Hz, 1H), 3.97- 3.89 (m, 1H), 3.74 (s, 3H), 3.52 (dd, J = 8.4, 6.9 Hz, 1H), 3.25 (dd, J = 7.9, 5.8 Hz, 2H), 3.10 (dd, J = 8.4, 6.2 Hz, 1H), 2.19 (dq, J = 12.5, 6.0 Hz, 1H), 1.88 (dq, J = 12.1, 7.4 Hz, 1H), 1.14-1.11 (m, 6H). | MS (ESI+) m/z 595.3 (M + H)⁺ |
| Compound 29 | 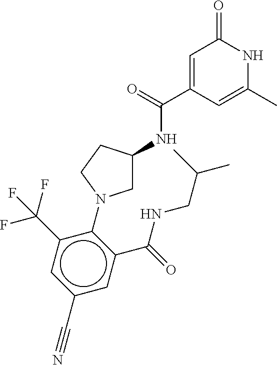 | ¹H NMR (600 MHz, DMSO- d₆) δ ppm 11.80 (s, 1H), 8.73 (t, J = 5.8 Hz, 1H), 8.53 (d, J = 6.8 Hz, 1H), 8.22 (d, J = 2.1 Hz, 1H), 7.96 (d, J = 2.1 Hz, 1H), 6.58 (d, J = 1.6 Hz, 1H), 6.35-6.25 (m, 1H), 4.43 (h, J = 6.1 Hz, 1H), 3.56 (dd, J = 9.5, 6.4 Hz, 1H), 3.46-3.39 (m, 2H), 3.20 (dd, J = 9.4, 4.8 Hz, 1H), 3.05 (dq, J = 13.0, 7.2 Hz, 2H), 2.20 (s, 3H), 2.17-2.10 (m, 1H), 2.00- 1.92 (m, 1H), 1.81 (hept, J = 6.7 Hz, 1H), 0.88 (dd, J = 6.7, 1.7 Hz, 6H). | MS (APCI+) m/z 490 (M + H)⁺ |
| Compound 30 | 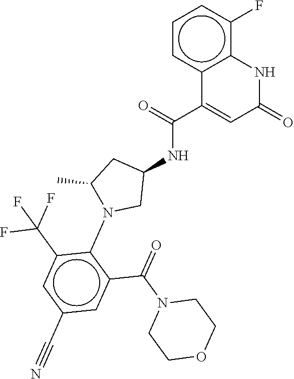 | ¹H NMR (400 MHz, DMSO- d₆) δ ppm 11.92 (s, 1H), 8.50 (d, J = 6.9 Hz, 1H), 8.24 (d, J = 2.0 Hz, 1H), 8.05 (d, J = 2.0 Hz, 1H), 7.67-7.35 (m, 2H), 7.18 (td, J = 8.1, 5.1 Hz, 1H), 6.74 (s, 1H), 4.49 (s, 1H), 3.90-3.67 (m, 3H), 3.54- 3.09 (m, 8H), 2.34-2.12 (m, 1H), 1.80 (ddd, J = 12.6, 8.5, 5.9 Hz, 1H), 0.98 (d, J = 5.9 Hz, 3H). | MS (APCI+) m/z 572 (M + H)⁺ |
| Compound 31 | 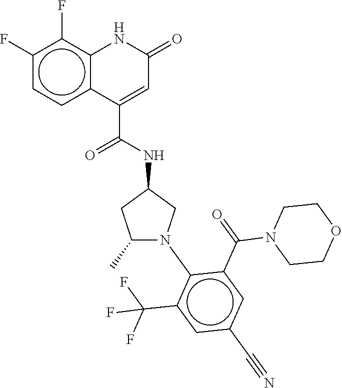 | ¹H NMR (400 MHz, DMSO- d₆) δ ppm 12.19 (s, 1H), 8.74 (dd, J = 174.6, 6.2 Hz, 1H), 8.24 (d, J = 2.1 Hz, 1H), 8.01 (dd, J = 36.3, 2.1 Hz, 1H), 7.61 (ddd, J = 9.0, 5.2, 1.9 Hz, 1H), 7.25 (td, J = 9.9, 7.4 Hz, 1H), 6.73 (s, 1H), 4.48 (s, 1H), 4.13-3.38 (m, 10H), 3.26-3.10 (m, 1H), 2.26 (dd, J = 12.7, 6.4 Hz, 1H), 1.91- 1.69 (m, 1H), 0.98 (d, J = 5.9 Hz, 3H). | MS (APCI+) m/z 590 (M + H)⁺ |
| Compound 32 | 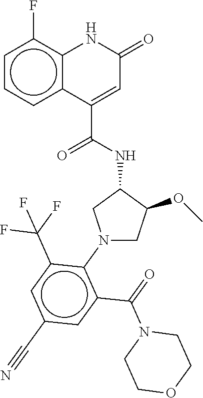 | ¹H NMR (600 MHz, DMSO- d₆) δ ppm 11.97 (s, 1H), 9.04 (d, J = 6.3 Hz, 0.5H), 8.74 (d, J = 6.6 Hz, 0.5H), 8.20 (d, J = 2.1 Hz, 0.5H), 8.19 (d, J = 2.1 Hz, 0.5H), 7.97 (d, J = 2.1 Hz, 0.5H), 7.92 (d, J = 2.1 Hz, 0.5H), 7.56 (d, J = 8.2 Hz, 0.5H), 7.53 (d, J = 8.2 Hz, 0.5H), 7.49-7.44 (m, 1H), 7.22-7.16 (m, 1H), 6.67 (s, 0.5H), 6.60 (s, 0.5H), 4.41- 4.34 (m, 1H), 4.08-4.04 (m, 1H), 3.78-3.42 (m, 8H), 3.40-3.19 (m, 7H). | MS (ESI+) m/z 588.4 (M + H)⁺ |
| Compound 33 | 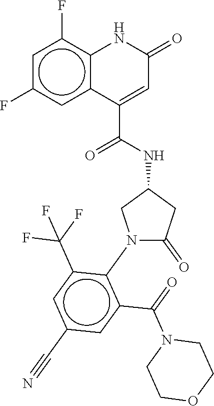 | ¹H NMR (600 MHz, DMSO- d₆) δ ppm 12.09 (s, 1H), 9.33 (d, J = 5.6 Hz, 0.5H), 9.06 (d, J = 5.7 Hz, 0.5H), 8.50 (dd, J = 9.2, 1.9 Hz, 1H), 8.40 (dd, J = 12.3, 1.9 Hz, 1H), 7.63 (dddd, J = 11.2, 8.2, 5.2, 2.7 Hz, 1H), 7.55-7.49 (m, 0.5H), 7.48-7.43 (m, 0.5H), 6.91 (s, 0.5H), 6.77 (s, 0.5H), 4.65 (tq, J = 11.3, 5.5 Hz, 1H), 4.10 (dd, J = 9.8, 6.5 Hz, 0.5H), 3.98 (dd, J = 9.8, 6.6 Hz, 0.5H), 3.91-3.81 (m, 1H), 3.79-3.73 (m, 1H), 3.67- 3.16 (m, 6H), 3.10-3.02 (m, 1H), 2.90 (dd, J = 17.1, 8.0 Hz, 0.5H), 2.79 (dd, J = 17.2, 8.2 Hz, 0.5H), 2.61 (ddd, J = 27.7, 17.2, 4.9 Hz, 1H). | MS (APCI+) m/z 590.29 (M + H)⁺ |
| Compound 34 | 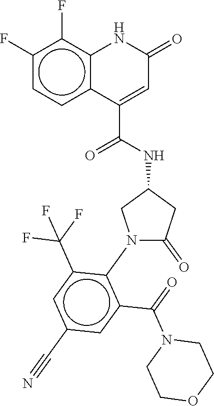 | ¹H NMR (500 MHz, DMSO- d₆) δ ppm 12.26 (s, 1H), 9.34 (d, J = 5.7 Hz, 0.5H), 9.07 (d, J = 5.8 Hz, 0.5H), 8.51 (dd, J = 8.0, 1.8 Hz, 1H), 8.41 (dd, J = 10.0, 1.9 Hz, 1H), 7.73- 7.62 (m, 1H), 7.34-7.24 (m, 1H), 6.80 (s, 0.5H), 6.65 (s, 0.5H), 4.66 (tdd, J = 14.3, 11.7, 11.3, 5.3 Hz, 1H), 4.11 (dd, J = 9.7, 6.6 Hz, 0.5H), 4.00 (dd, J = 9.8, 6.6 Hz, 0.5H), 3.91-3.16 (m, 8H), 3.11-3.04 (m, 1H), 2.92 (dd, J = 17.1, 8.0 Hz, 0.5H), 2.80 (dd, J = 17.1, 8.1 Hz, 0.5H), 2.71-2.55 (m, 1H). | MS (APCI+) m/z 590.30 (M + H)⁺ |
| Compound 35 | 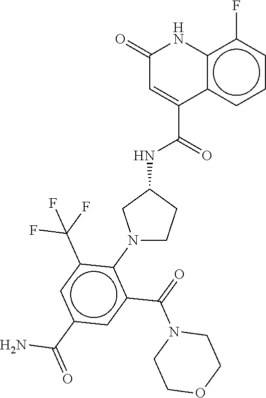 | ¹H NMR (600 MHz, DMSO- d₆) δ ppm 11.95 (s, 1H), 9.01- 8.81 (m, 1H), 8.24-8.21 (m, 1H), 8.17 (s, 1H), 7.97- 7.94 (m, 1H), 7.60-7.51 (m, 2H), 7.49-7.43 (m, 1H), 7.21-7.16 (m, 1H), 6.66- 6.60 (m, 1H), 4.50-4.41 (m, 1H), 3.72-3.54 (m, 5H), 3.54-3.45 (m, 1H), 3.41- 3.18 (m, 6H), 2.29-2.18 (m, 1H), 2.03-1.91 (m, 1H); ¹H NMR (400 MHz, DMSO-d6, 90° C.) δ ppm 11.48 (bs, 1H), 8.57 (bs, 1H), 8.20 (d, J = 2.2 Hz, 1H), 7.92 (d, J = 2.2 Hz, 1H), 7.56 (bs, 2H), 7.56 (dd, J = 8.1, 1.3 Hz, 1H), 7.39 (ddd, J = 11.1, 8.1, 1.3 Hz, 1H), 7.16 (ddd, J = 8.1, 8.1, 5.1 Hz, 1H), 6.61 (s, 1H), 4.53- 4.44 (m, 1H), 3.77-3.49 (m, 6H), 3.41-3.17 (m, 6H), 2.31-2.21 (m, 1H), 2.03- | MS (APCI+) m/z 576 (M + H)⁺ |
| | | 1.94 (m, 1H). | |
| Compound 36 | 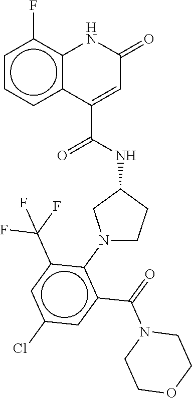 | ¹H NMR (400 MHz, DMSO- d₆) δ ppm 11.96 (s, 1H), 8.88 (dd, J = 59.2, 6.5 Hz, 1H), 7.76 (dd, J = 24.3, 2.7 Hz, 2H), 7.60-7.38 (m, 2H), 7.26-7.11 (m, 1H), 6.62 (d, J = 14.6 Hz, 1H), 4.43 (p, J = 6.5 Hz, 1H), 3.73-3.55 (m, 5H), 3.55-3.41 (m, 3H), 3.24-3.11 (m, 2H), 2.21 (ddt, J = 19.7, 13.7, 6.4 Hz, 1H), 1.99 (s, 2H). | MS (ESI+) m/z 567 (M + H)⁺ |
| Compound 37 | 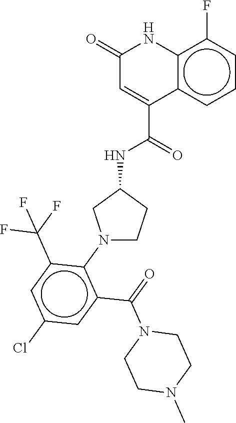 | ¹H NMR (400 MHz, DMSO- d₆) δ ppm 11.96 (s, 1H), 8.87 (dd, J = 61.4, 6.5 Hz, 1H), 7.82-7.63 (m, 2H), 7.58- 7.42 (m, 2H), 7.19 (td, J = 8.1, 5.1 Hz, 1H), 6.62 (d, J = 11.6 Hz, 1H), 4.43 (t, J = 7.7 Hz, 1H), 3.67 (s, 1H), 3.49 (ddd, J = 25.9, 8.4, 6.5 Hz, 3H), 3.22-3.10 (m, 2H), 2.87 (s, 6H), 2.40 (d, J = 6.3 Hz, 2H), 2.29 (t, J = 5.2 Hz, 2H), 1.92 (ddd, J = 18.9, 12.4, 6.5 Hz, 1H). | MS (ESI+) m/z 580 (M + H)⁺ |
| Compound 38 | 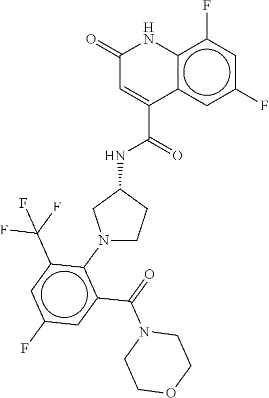 | ¹H NMR (400 MHz, DMSO- d₆) δ ppm 12.06 (s, 1H), 8.97 (d, J = 6.4 Hz, 0.5H), 8.84 (d, J = 6.3 Hz, 0.5H), 7.69-7.54 (m, 3H), 7.44-7.35 (m, 1H), 6.76 (s, 0.5H), 6.73 (s, 0.5H), 4.47-4.36 (m, 1H), 3.74- 3.03 (m, 12H), 2.28-2.13 (m, 1H), 1.99-1.85 (m, 1H). | MS (ESI+) m/z 569.3 (M + H)⁺ |
| Compound 39 | 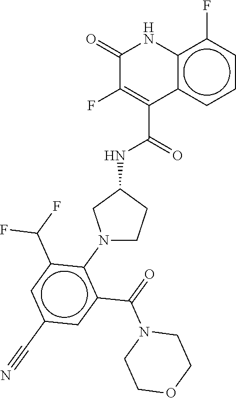 | ¹H NMR (600 MHz, DMSO- d₆) δ ppm 12.58 (s, 1H), 9.28 (d, J = 6.5 Hz, 0.5H), 9.19 (d, J = 6.6 Hz, 0.5H), 8.02-7.99 (m, 0.5H), 7.99 (d, J = 2.1 Hz, 0.5H), 7.82 (d, J = 2.1 Hz, 0.5H), 7.79 (d, J = 2.1 Hz, 0.5H), 7.52-7.45 (m, 1H), 7.36-7.14 (m, 3H), 4.63- 4.51 (m, 1H), 3.75-3.22 (m, 11.5H), 3.13 (dd, J = 9.5, 3.9 Hz, 0.5H), 2.37-2.23 (m, 1H), 2.04-1.89 (m, 1H). | MS (ESI+) m/z 558.3 (M + H)⁺ |
| Compound 40 | 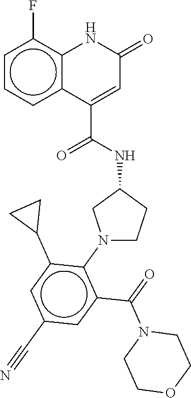 | ¹H NMR (400 MHz, DMSO- d₆) δ ppm 11.95 (s, 1H), 8.97 (dd, J = 53.2, 6.4 Hz, 1H), 7.53 (t, J = 7.9 Hz, 1H), 7.46 (dddd, J = 11.0, 8.1, 2.9, 1.2 Hz, 1H), 7.41 (dd, J = 2.9, 2.0 Hz, 1H), 7.35 (q, J = 2.5, 2.1 Hz, 1H), 7.18 (tdd, J = 8.1, 5.1, 1.0 Hz, 1H), 6.61 (d, J = 16.0 Hz, 1H), 4.48 (dt, J = 12.8, 6.3 Hz, 1H), 3.75-3.54 (m, 5H), 3.54-3.45 (m, 1H), 3.45-3.35 (m, 1H), 3.26- 3.19 (m, 1H), 2.26 (dp, J = 20.1, 6.6 Hz, 1H), 2.09 (dt, J = 8.4, 4.2 Hz, 1H), 1.98 (ddd, J = 19.9, 12.5, 6.8 Hz, 1H), 1.10-0.79 (m, 3H), 0.71 (ddt, J = 14.0, 9.3, 4.8 Hz, 1H). | MS (ESI+) m/z 530 (M + H)⁺ |
| Compound 41 | 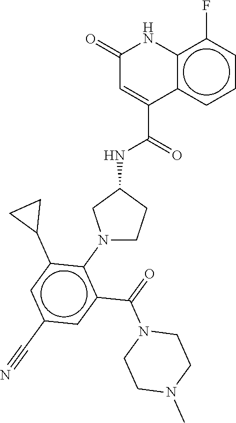 | ¹H NMR (400 MHz, DMSO- d₆) δ ppm 11.95 (s, 1H), 8.96 (dd, J = 51.1, 6.5 Hz, 1H), 7.53 (dd, J = 8.2, 3.4 Hz, 1H), 7.46 (dddd, J = 10.9, 8.1, 2.6, 1.2 Hz, 1H), 7.40-7.29 (m, 2H), 7.18 (tdd, J = 8.0, 5.1, 2.8 Hz, 1H), 6.60 (d, J = 13.9 Hz, 1H), 4.47 (td, J = 12.4, 6.2 Hz, 1H), 3.59 (dddd, J = 47.5, 26.7, 8.7, 5.8 Hz, 4H), 3.44-3.35 (m, 1H), 3.23 (ddd, J = 13.7, 7.5, 3.2 Hz, 2H), 2.41-2.30 (m, 2H), 2.30-2.23 (m, 2H), 2.18 (d, J = 14.8 Hz, 3H), 2.10 (ddt, J = 8.3, 5.7, 2.9 Hz, 1H), 1.09- 0.82 (m, 3H), 0.71 (ddt, J = 16.4, 11.5, 4.2 Hz, 1H). | MS (ESI+) m/z 543 (M + H)⁺ |
| Compound 42 | 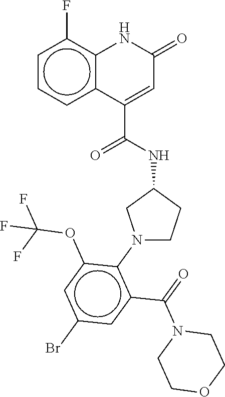 | ¹H NMR (400 MHz, DMSO- d₆) d 11.96 (s, 1H), 8.98 (dd, J = 26.7, 6.1 Hz, 1H), 7.55- 7.42 (m, 3H), 7.35 (dd, J = 3.9, 2.4 Hz, 1H), 7.18 (tt, J = 8.1, 5.0 Hz, 1H), 6.62 (d, J = 7.3 Hz, 1H), 4.43 (dh, J = 12.3, 6.1 Hz, 1H), 3.68-3.35 (m, 9H), 3.29-3.17 (m, 3H), 2.21 (dq, J = 13.0, 6.7 Hz, 1H), 2.05-1.91 (m, 1H). | MS (APCI+) m/z 627/629 |
| Compound 43 | 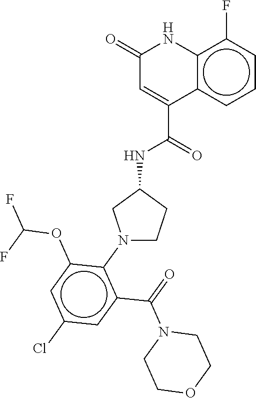 | ¹H NMR (400 MHz, DMSO- d₆) δ ppm 11.95 (s, 1H), 8.94 (dd, J = 33.6, 6.3 Hz, 1H), 7.53 (t, J = 8.4 Hz, 1H), 7.46 (dd, J = 11.1, 8.0 Hz, 1H), 7.25 (dd, J = 4.1, 2.5 Hz, 1H), 7.22-7.15 (m, 1H), 7.20 (td, J = 73.9, 3.9 Hz, 1H), 7.12 (dd, J = 3.3, 2.4 Hz, 1H), 6.62 (d, J = 7.4 Hz, 1H), 4.44 (dd, J = 6.3, 2.8 Hz, 1H), 3.69-3.42 (m, 7H), 3.38 (td, J = 8.2, 7.6, 6.1 Hz, 2H), 3.24-3.16 (m, 3H), 2.28-2.15 (m, 1H), 2.02- 1.88 (m, 1H) | MS (APCI+) m/z 565/567 |
| Compound 44 | 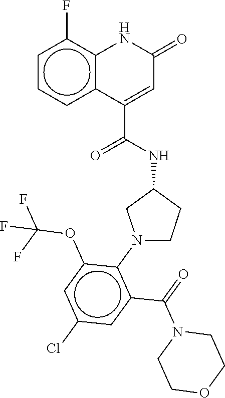 | ¹H NMR (600 MHz, DMSO- d₆) d 11.95 (s, 1H), 8.97 (dd, J = 41.1, 6.1 Hz, 1H), 7.55- 7.49 (m, 1H), 7.48-7.42 (m, 2H), 7.26 (dd, J = 5.2, 2.5 Hz, 1H), 7.18 (qd, J = 7.9, 5.0 Hz, 1H), 6.62 (d, J = 11.3 Hz, 1H), 4.44 (dq, J = 16.6, 6.1 Hz, 1H), 3.67-3.48 (m, 6H), 3.47-3.34 (m, 3H), 3.29-3.17 (m, 3H), 2.26-2.17 (m, 1H), 2.03-1.93 (m, 1H). | MS (APCI+) m/z 583/585 |
| Compound 45 | 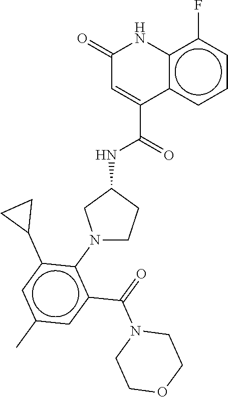 | ¹H NMR (400 MHz, DMSO- d₆, 90° C.) δ ppm 8.53 (s, 1H), 7.59-7.53 (m, 1H), 7.39 (ddd, J = 11.0, 8.1, 1.2 Hz, 1H), 7.16 (td, J = 8.1, 5.1 Hz, 1H), 6.70 (t, J = 1.3 Hz, 1H), 6.68 (d, J = 2.1 Hz, 1H), 6.58 (s, 1H), 4.50 (h, J = 6.2 Hz, 1H), 4.20 (s, 7H), 3.57 (dd, J = 8.9, 6.4 Hz, 1H), 3.35 (td, J = 8.0, 5.8 Hz, 1H), 3.27 (td, J = 8.1, 5.8 Hz, 1H), 3.23 (s, 2H), 3.18 (dd, J = 8.9, 4.9 Hz, 1H), 2.32-2.21 (m, 1H), 2.21 (s, 3H), 2.13 (tt, J = 8.4, 5.4 Hz, 1H), 1.95 (dq, J = 12.9, 6.0 Hz, 1H), 1.00-0.87 (m, 2H), 0.65 (s, 2H). | MS (ESI+) m/z 519 (M + H)⁺ |
| Compound 46 | 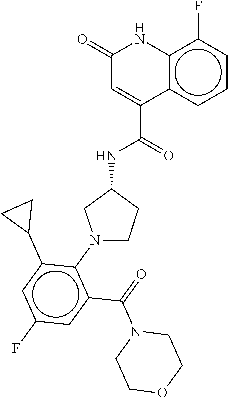 | ¹H NMR (500 MHz, DMSO- d₆) δ ppm 11.93 (s, 1H), 8.89 (dd, J = 75.5, 6.7 Hz, 1H), 7.54 (t, J = 8.4 Hz, 1H), 7.46 (t, J = 9.5 Hz, 1H), 7.19 td, J = 8.2, 5.1 Hz, 1H), 6.82 (ddd, J = 8.2, 3.0, 1.7 Hz, 1H), 6.69 (dt, J = 10.2, 2.6 Hz, 1H), 6.60 (d, J = 21.7 Hz, 1H), 4.48 (q, J = 6.9 Hz, 1H), 3.71- 3.44 (m, 7H), 3.23-3.09 (m, 3H), 2.32-2.12 (m, 2H), 1.92 (ddt, J = 19.6, 13.7, 6.2 Hz, 1H), 1.26 (d, J = 20.0 Hz, 2H), 1.09-0.87 (m, 3H), 0.73 (ddt, J = 50.9, 9.7, 5.0 Hz, 2H). | MS (APCI+) m/z 523 (M + H)⁺ |
| Compound 47 | 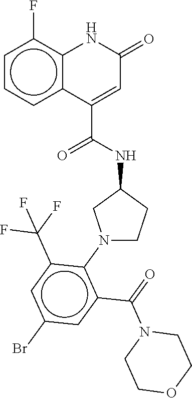 | ¹H NMR (600 MHz, DMSO- d₆) δ ppm 11.95 (s, 1H), 8.88 (dd, J = 90.1, 6.4 Hz, 1H), 7.89 (d, J = 2.4 Hz, 1H), 7.84 (dd, J = 4.9, 2.4 Hz, 1H), 7.53 (t, J = 8.2 Hz, 1H), 7.49- 7.44 (m, 1H), 7.19 (tdd, J = 8.2, 5.0, 3.3 Hz, 1H), 6.62 (d, J = 21.8 Hz, 1H), 4.47-4.39 (m, 1H), 3.72-3.43 (m, 7H), 3.31-3.07 (m, 5H), 2.27- 2.16 (m, 1H), 1.98-1.86 (m, 1H). | MS (AA+) m/z 611.3, 613.3 |
| Compound 48 | 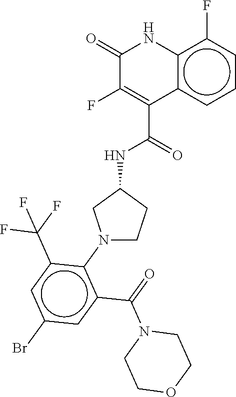 | ¹H NMR (400 MHz, DMSO- d₆, 90° C.) δ ppm 12.15 (s, 1H), 8.79 (s, 1H), 7.83 (d, J = 2.5 Hz, 1H), 7.73 (d, J = 2.4 Hz, 1H), 7.45 7.35 (m, 1H), 7.31 (d, J = 7.5 Hz, 1H), 7.23 (td, J = 8.0, 4.9 Hz, 1H), 4.51 (q, J = 6.8 Hz, 1H), 3.60 (s, 7H), 3.30 3.24 (m, 5H), 2.27 (dt, J = 13.2, 6.6 Hz, 1H), 1.94 1.86 (m, 1H). | MS (ESI+) m/z 629 (M + H)⁺ |
| Compound 49 | 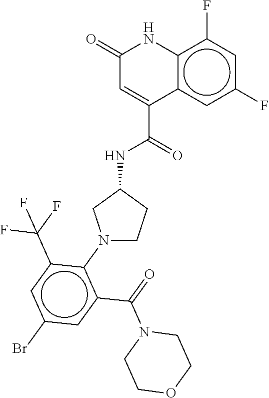 | ¹H NMR (400 MHz, DMSO- d₆) δ ppm 12.06 (s, 1H), 8.98 (d, J = 6.3 Hz, 0.55H), 8.84 (d, J = 6.4 Hz, 0.45H), 7.89 (s, 0.45H), 7.89 (s, 0.55H), 7.85-7.82 (m, 1H), 7.65- 7.58 (m, 1H), 7.42-7.35 (m, 1H), 6.77 (s, 0.55H), 6.73 (s, 0.45H), 4.48-4.36 (m, 1H), 3.75-3.40 (m, 7H), 3.29- 3.05 (m, 5H), 2.29-2.13 (m, 1H), 2.02-1.86 (m, 1H). | LC/MS (ESI+) m/z 629, 631 (M + H)⁺ |
| Compound 50 | 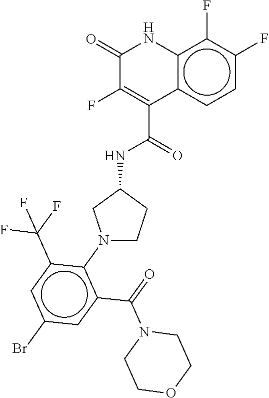 | ¹H NMR (400 MHz, DMSO- d₆) δ ppm 12.42 (s, 1H), 8.80 (s, 1H), 7.83 (d, J = 2.4 Hz, 1H), 7.73 (d, J = 2.4 Hz, 1H), 7.32 (ddd, J = 9.1, 5.3, 1.6 Hz, 1H), 7.32-7.21 (m, 1H), 4.50 (h, J = 6.8 Hz, 1H), 3.61 (s, 7H), 3.27 (s, 4H), 3.11 (s, 1H), 2.26 (dt, J = 13.0, 6.5 Hz, 1H), 1.88 (dd, J = 12.8, 6.7 Hz, 1H). | MS (ESI+) m/z 647 (M + H)⁺ |
| Compound 51 | 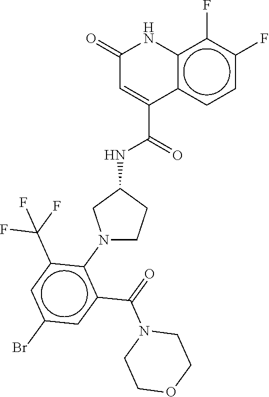 | ¹H NMR (400 MHz, DMSO- d₆) δ ppm 12.21 (s, 1H), 8.97 (d, J = 6.4 Hz, 0.45H), 8.82 (d, J = 6.5 Hz, 0.55H), 7.89 (s, 0.45H), 7.89 (s, 0.55H), 7.85-7.82 (m, 1H), 7.61- 7.54 (m, 1H), 7.30-7.21 (m, 1H), 6.62 (s, 0.55H), 6.59 (s, 0.45H), 4.47-4.36 (m, 1H), 3.75-3.42 (m, 7H), 3.30- 3.05 (m, 5H), 2.27-2.13 (m, 1H), 2.01-1.84 (m, 1H). | LC/MS (ESI+) m/z 629, 631 (M + H)⁺ |
| Compound 52 | 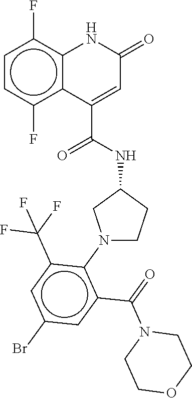 | ¹H NMR (400 MHz, DMSO- d₆, 90° C.) δ ppm 8.36 (s, 1H), 7.82 (d, J = 2.4 Hz, 1H), 7.72 (d, J = 2.4 Hz, 1H), 7.42 (ddd, J = 10.3, 9.0, 4.3 Hz, 1H), 6.94 (ddd, J = 10.8, 9.0, 3.8 Hz, 1H), 6.46 (s, 1H), 4.40 (q, J = 6.7 Hz, 1H), 3.65 (s, 2H), 3.60 (s, 4H), 3.49 (t, J = 7.6 Hz, 2H), 3.09 (s, 2H), 2.26-2.16 (m, 1H), 1.89 (s, 1H). | MS (ESI+) m/z 629 (M + H)⁺ |
| Compound 53 | 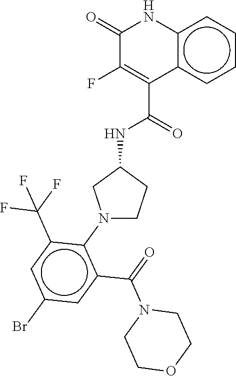 | ¹H NMR (400 MHz, DMSO- d₆, 90° C.) δ ppm 12.12 (s, 1H), 8.74 (s, 1H), 7.83 (d, J = 2.4 Hz, 1H), 7.73 (d, J = 2.5 Hz, 1H), 7.55-7.45 (m, 2H), 7.39 (d, J = 8.1 Hz, 1H), 7.23 (t, J = 7.8 Hz, 1H), 4.51 (q, J = 6.6 Hz, 1H), 3.61-3.57 (m, 7H), 3.27 (s, 4H), 3.16- 3.08 (m, 1H), 2.27 (dd, J = 12.5, 6.0 Hz, 1H), 1.90 (s, 1H). | MS (ESI+) m/z 611 (M + H)⁺ |
| Compound 54 | 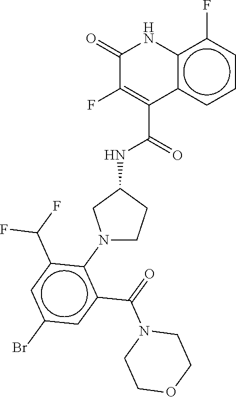 | ¹H NMR (400 MHz, DMSO- d₆) δ ppm 12.57 (s, 1H), 9.25 (d, J = 6.9 Hz, 0.5H), 9.17 (d, J = 6.9 Hz, 0.5H), 7.74-7.70 (m, 1H), 7.67-7.62 (m, 1H), 7.53-7.44 (m, 1H), 7.37- 7.01 (m, 3H), 4.63-4.49 (m, 1H), 3.77-3.42 (m, 4H), 3.40- 3.14 (m, 7H), 3.11-2.99 (m, 1H), 2.39-2.21 (m, 1H), 1.97-1.79 (m, 1H). | MS (ESI+) m/z 611.3 (M + H)⁺ |
| Compound 55 | 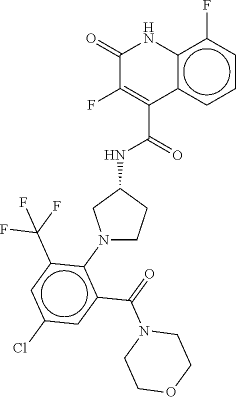 | ¹H NMR (400 MHz, DMSO- d₆, 90° C.) δ ppm 12.14 (s, 1H), 8.79 (s, 1H), 7.72 (d, J = 2.6 Hz, 1H), 7.62 (d, J = 2.6 Hz, 1H), 7.40 (ddd, J = 11.0, 8.0, 1.3 Hz, 1H), 7.31 (dt, J = 8.1, 1.1 Hz, 1H), 7.23 (td, J = 8.1, 4.9 Hz, 1H), 4.51 (h, J = 6.9 Hz, 1H), 3.60 (s, 7H), 3.27 (s, 4H), 3.11 (s, 1H), 2.28 (dq, J = 13.0, 6.7 Hz, 1H), 1.90 (dt, J = 12.9, 7.1 Hz, 1H). | MS (ESI+) m/z 585 (M + H)⁺ |
| Compound 56 | 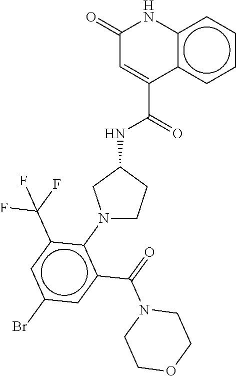 | ¹H NMR (400 MHz, DMSO- d₆, 90° C.) δ ppm 11.57 (s, 1H), 8.48 (s, 1H), 7.83 (d, J = 2.5 Hz, 1H), 7.71 (dd, J = 10.5, 2.0 Hz, 2H), 7.49 (ddd, J = 8.4, 7.1, 1.4 Hz, 1H), 7.35 (dd, J = 8.4, 1.2 Hz, 1H), 7.16 (ddd, J = 8.3, 7.1, 1.2 Hz, 1H), 6.51 (s, 1H), 4.46 (q, J = 6.5 Hz, 1H), 3.62 (s, 4H), 3.54 (s, 3H), 3.26 (s, 5H), 2.24 (dt, J = 12.9, 6.7 Hz, 1H), 1.95 (s, 1H). | MS (ESI+) m/z 593 (M + H)⁺ |
| Compound 57 | 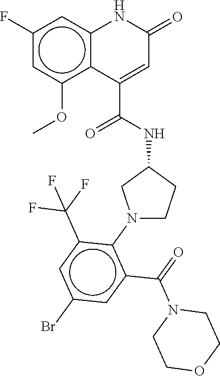 | ¹H NMR (400 MHz, DMSO- d₆, 90° C.) δ ppm 11.59 (s, 1H), 8.01 (s, 1H), 7.82 (d, J = 2.4 Hz, 1H), 7.72 (d, J = 2.4 Hz, 1H), 6.71 (dd, J = 10.0, 2.4 Hz, 1H), 6.64 (dd, J = 11.3, 2.4 Hz, 1H), 6.13 (s, 1H), 4.40 (h, J = 6.8 Hz, 1H), 3.78 (s, 3H), 3.65 (s, 1H), 3.62-3.54 (m, 3H), 3.54- 3.45 (m, 1H), 3.26 (s, 5H), 3.09 (s, 1H), 2.21 (dd, J = 12.6, 6.4 Hz, 1H), 1.89 (s, 1H). | MS (ESI+) m/z 641 (M + H)⁺ |
| Compound 58 | 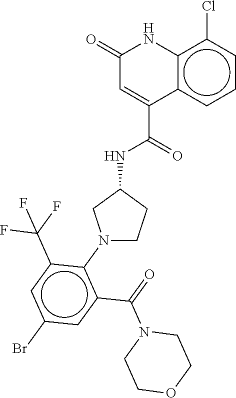 | ¹H NMR (500 MHz, DMSO- d₆) δ ppm 11.21 (s, 1H), 9.00- 8.82 (m, 1H), 7.90 (d, J = 2.4 Hz, 1H), 7.85-7.83 (m, 1H), 7.74-7.67 (m, 2H), 7.25-7.20 (m, 1H), 6.68- 6.61 (m, 1H), 4.48-4.39 (m, 1H), 3.72-3.43 (m, 7H), 3.31-3.07 (m, 5H), 2.28- 2.16 (m, 1H), 1.99-1.85 (m, 1H). | |
| Compound 59 | 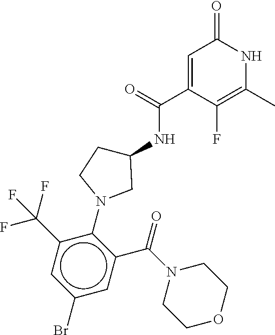 | ¹H NMR (500 MHz, DMSO- d₆) δ ppm 11.40 (s, 1H), 8.60 (dd, J = 72.2, 6.6 Hz, 1H), 7.88 (d, J = 2.5 Hz, 1H), 7.83 (dd, J = 6.2, 2.4 Hz, 1H), 6.31 (d, J = 15.6 Hz, 1H), 4.34 (dq, J = 13.9, 6.6 Hz, 1H), 3.82-3.52 (m, 5H), 3.52- 3.38 (m, 2H), 3.25-3.12 (m, 3H), 3.04 (ddd, J = 24.9, 8.7, 5.9 Hz, 1H), 2.23 (t, J = 2.3 Hz, 3H), 2.20-2.09 (m, 1H), 1.95-1.75 (m, 1H) | MS (ESI+) m/z 575/577 (M + H)⁺ |
| Compound 60 | 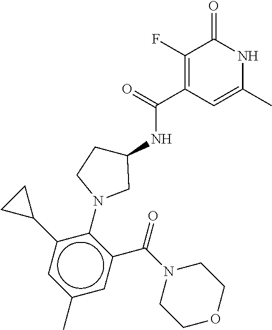 | ¹H NMR (600 MHz, DMSO- d₆) δ ppm 12.20 (s, 1H), 8.63 (dd, J = 78.2, 6.9 Hz, 1H), 6.79-6.55 (m, 2H), 5.96 (dd, J = 15.7, 4.5 Hz, 1H), 4.39 (p, J = 6.0 Hz, 1H), 3.66-3.37 (m, 7H), 3.30-3.05 (m, 6H), 2.26-2.10 (m, 8H), 1.85 (dddd, J = 18.8, 12.5, 8.0, 6.1 Hz, 1H), 1.01-0.86 (m, 2H), 0.72 (ddt, J = 13.1, 5.6, 3.8 Hz, 1H), 0.65-0.54 (m, 1H) | MS (ESI+) m/z 483 (M + H)⁺ |
| Compound 61 | 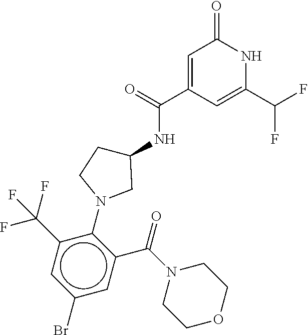 | ¹H NMR (400 MHz, DMSO- d₆, 90° C.) δ ppm 8.40 (s, 1H), 7.82 (d, J = 2.4 Hz, 1H), 7.73 (d, J = 2.5 Hz, 1H), 7.31 (s, 1H), 7.11 (d, J = 1.4 Hz, 1H), 6.76 (t, J = 54.8 Hz, 1H), 4.44 (p, J = 6.5 Hz, 1H), 3.88-3.71 (m, 3H), 3.65- 3.57 (m, 1H), 3.48 (t, J = 8.0 Hz, 2H), 3.27 (d, J = 23.1 Hz, 5H), 3.12 (s, 1H), 2.19 (dq, J = 13.4, 6.9 Hz, 1H), 1.99 (dq, J = 13.3, 6.8 Hz, 1H). | MS (ESI+) m/z 593 (M + H)⁺ |
| Compound 62 | 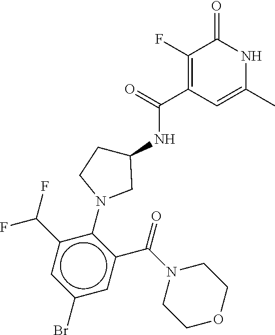 | ¹H NMR (500 MHz, DMSO- d₆) δ ppm 12.59 (s, 1H), 9.50 (d, J = 5.9 Hz, 0.5H), 9.33 (d, J = 6.3 Hz, 0.5H), 8.05-7.97 (m, 2H), 7.51-7.45 (m, 1H), 7.42 (d, J = 8.1 Hz, 0.5H), 7.35 (d, J = 8.1 Hz, 0.5H), 7.30-7.23 (m, 1H), 4.81- 4.64 (m, 1H), 4.15 (t, J = 8.4 Hz, 0.5H), 4.00 (dd, J = 9.6, 7.4 Hz, 0.5H), 3.88 (d, J = 13.3 Hz, 0.5H), 3.82-3.04 (m, 8.5H), 2.97-2.88 (m, 0.5H), 2.84 (dd, J = 17.2, 8.5 Hz, 0.5H), 2.56-2.51 (m, 0.5H), 2.46 (dd, J = 17.0, 7.2 Hz, 0.5H). | MS (ESI+) m/z 599.3 (M + H)⁺ |
| Compound 63 | 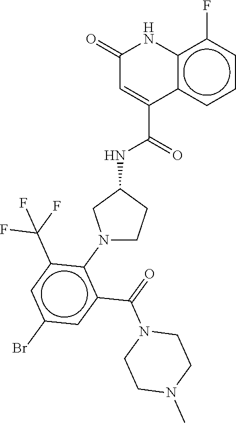 | ¹H NMR (600 MHz, DMSO- d₆) δ ppm 11.97 (s, 1H), 10.15-9.85 (m, 1H), 9.01- 8.87 (m, 1H), 7.94 (s, 2H), 7.54 (dd, J = 8.2, 4.1 Hz, 1H), 7.47 (ddd, J = 11.0, 8.1, 1.2 Hz, 1H), 7.19 (tt, J = 8.1, 4.9 Hz, 1H), 6.70-6.57 (m, 1H), 4.70-4.50 (m, 1H), 3.46-2.78 (m, 15H), 2.29-2.13 (m, 1H), 2.04-1.73 (m, 1H). | MS (APCI+) m/z 624 and 626 (M + H)⁺ |
| Compound 64 | 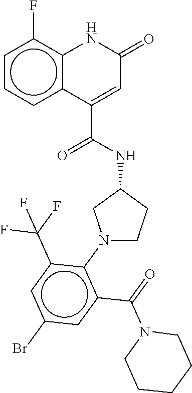 | ¹H NMR (400 MHz, DMSO- d₆, 90° C.) δ ppm 8.67-8.29 (m, 1H), 7.81 (d, J = 2.5 Hz, 1H), 7.65 (d, J = 2.4 Hz, 1H), 7.55 (d, J = 8.1 Hz, 1H), 7.44- 7.34 (m, 1H), 7.20-7.11 (m, 1H), 6.59 (s, 1H), 4.50- 4.41 (m, 1H), 3.68-3.42 (m, 3H), 3.19 (d, J = 5.2 Hz, 5H), 2.26-2.15 (m, 1H), 1.96- 1.92 (m, 1H), 1.56-1.51 (m, 6H). | MS (APCI+) m/z 609.4 (M + H)⁺ |
| Compound 65 | 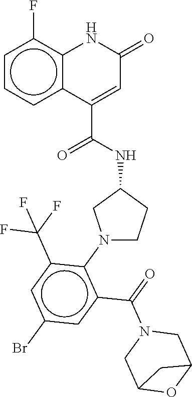 | ¹H NMR (400 MHz, DMSO- d₆, 90° C.) δ ppm 8.54 (s, 1H), 7.87-7.82 (m, 2H), 7.53 (dd, J = 8.3, 4.7 Hz, 1H), 7.44-7.34 (m, 1H), 7.20- 7.10 (m, 1H), 6.60-6.56 (m, 1H), 4.64-4.57 (m, 1H), 4.51-4.42 (m, 2H), 3.90- 3.81 (m, 1H), 3.67-3.48 (m, 3H), 3.46-3.11 (m, 5H), 2.27-2.16 (m, 1H), 2.00- 1.79 (m, 2H). | MS (APCI+) m/z 623.3 (M + H)⁺ |
| Compound 66 | 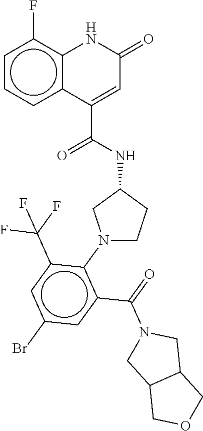 | ¹H NMR (400 MHz, DMSO- d₆, 90° C.) δ ppm 8.51 (d, J = 6.7 Hz, 1H), 7.82 (d, J = 2.5 Hz, 1H), 7.74 (d, J = 2.5 Hz, 1H), 7.55 (d, J = 8.3 Hz, 1H), 7.39 (ddd, J = 10.9, 8.1, 1.2 Hz, 1H), 7.20-7.11 (m, 1H), 6.60 (s, 1H), 4.50-4.44 (m, 1H), 3.79-3.65 (m, 3H), 3.61-3.38 (m, 5H), 3.35- 3.18 (m, 3H), 3.16-3.09 (m, 1H), 2.96-2.85 (m, 2H), 2.26-2.17 (m, 1H), 1.99- 1.88 (m, 1H). | MS (APCI+) m/z 637.2 (M + H)⁺ |
| Compound 67 | 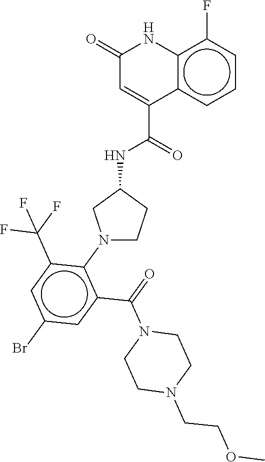 | ¹H NMR (400 MHz, DMSO- d₆, 90° C.) δ ppm 8.71-8.39 (m, 1H), 7.82 (d, J = 2.5 Hz, 1H), 7.66 (d, J = 2.4 Hz, 1H), 7.55 (d, J = 8.2 Hz, 1H), 7.44- 7.34 (m, 1H), 7.21-7.11 (m, 1H), 6.60 (s, 1H), 4.50- 4.41 (m, 1H), 3.56 (s, 3H), 3.43 (t, J = 5.8 Hz, 2H), 3.22 (s, 10H), 2.54-2.36 (m, 4H), 2.27-2.17 (m, 1H), 1.94 (s, 1H). | MS (APCI+) m/z 668.4 (M + H)⁺ |
| Compound 68 | 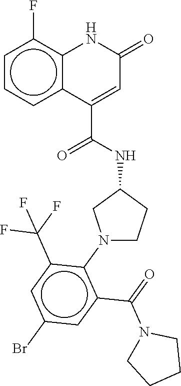 | ¹H NMR (400 MHz, DMSO- d₆, 90° C.) δ ppm 8.53 (d, J = 6.7 Hz, 1H), 7.81 (d, J = 2.5 Hz, 1H), 7.74 (d, J = 2.4 Hz, 1H), 7.54 (d, J = 8.1 Hz, 1H), 7.39 (ddd, J = 11.1, 8.1, 1.2 Hz, 1H), 7.15 (td, J = 8.1, 5.1 Hz, 1H), 6.60 (s, 1H), 4.51- 4.42 (m, 1H), 3.51 (dd, J = 8.9, 6.6 Hz, 1H), 3.47-3.42 (m, 2H), 3.30-3.16 (m, 5H), 2.28-2.15 (m, 1H), 1.99- 1.89 (m, 1H), 1.89-1.78 (m, 4H). | MS (APCI+) m/z 595.4 (M + H)⁺ |
| Compound 69 | 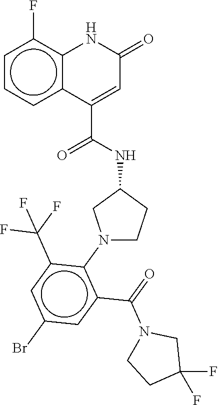 | ¹H NMR (400 MHz, DMSO- d₆, 90° C.) δ ppm 8.56-8.50 (m, 1H), 7.87-7.78 (m, 2H), 7.54 (d, J = 8.1 Hz, 1H), 7.39 (ddd, J = 11.0, 8.1, 1.2 Hz, 1H), 7.20-7.10 (m, 1H), 6.60 (s, 1H), 4.51-4.42 (m, 1H), 3.89 (t, J = 13.3 Hz, 1H), 3.73-3.69 (m, 1H), 3.55-3.46 (m, 2H), 3.38- 3.19 (m, 2H), 3.16-3.10 (m, 2H), 2.47-2.39 (m, 2H), 2.22 (dq, J = 13.2, 6.9 Hz, 1H), 2.00-1.88 (m, 1H). | MS (APCI+) m/z 631.3 (M + H)⁺ |
| Compound 70 | 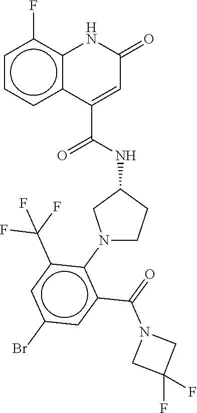 | ¹H NMR (400 MHz, DMSO- d₆, 90° C.) δ ppm 8.56 (d, J = 6.7 Hz, 1H), 7.93-7.85 (m, 2H), 7.55 (d, J = 8.2 Hz, 1H), 7.44-7.34 (m, 1H), 7.20- 7.10 (m, 1H), 6.60 (s, 1H), 4.57-4.38 (m, 5H), 3.52 (dd, J = 8.7, 6.7 Hz, 1H), 3.34- 3.28 (m, 1H), 3.27-3.21 (m, 1H), 3.16 (dd, J = 8.8, 5.6 Hz, 1H), 2.31-2.18 (m, 1H), 2.04-1.90 (m, 1H). | MS (APCI+) m/z 617.3 (M + H)⁺ |
| Compound 71 | 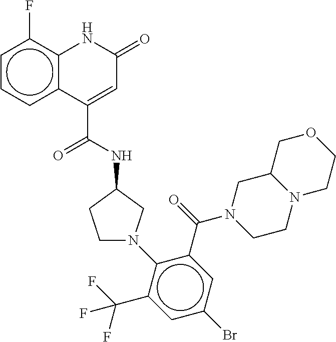 | ¹H NMR (400 MHz, DMSO- d₆, 90° C.) δ ppm 8.74-8.34 (m, 1H), 7.82 (s, 1H), 7.71- 7.67 (m, 1H), 7.56 (d, J = 8.3 Hz, 1H), 7.44-7.35 (m, 1H), 7.21-7.11 (m, 1H), 6.61 (s, 1H), 4.58-4.33 (m, 2H), 3.85-3.41 (m, 6H), 3.31 (s, 6H), 2.65-2.60 (m, 1H), 2.22 (s, 4H), 2.00-1.91 (m, 1H). | MS (APCI+) m/z 666.4 (M + H)⁺ |
| Compound 72 | 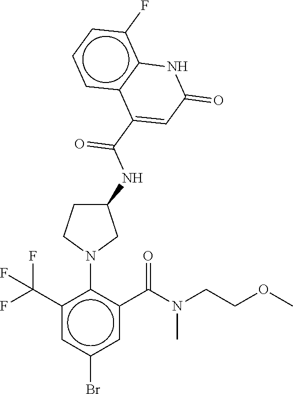 | ¹H NMR (400 MHz, DMSO- d₆, 90° C.) δ ppm 8.53-8.49 (m, 1H), 7.82 (s, 1H), 7.69- 7.60 (m, 1H), 7.55 (d, J = 8.2 Hz, 1H), 7.47-7.33 (m, 1H), 7.20-7.11 (m, 1H), 6.60 (s, 1H), 4.46 (s, 1H), 3.68-3.42 (m, 5H), 3.37-3.10 (m, 7H), 2.88-2.83 (m, 2H), 2.29- 2.16 (m, 1H), 1.97-1.88 (m, 1H). | MS (APCI+) m/z 613.4 (M + H)⁺ |
| Compound 73 | 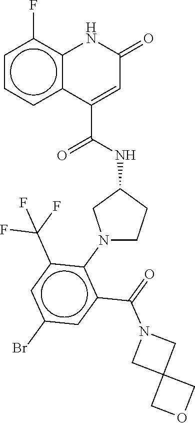 | ¹H NMR (400 MHz, DMSO- d₆, 90° C.) δ ppm 8.54 (d, J = 6.6 Hz, 1H), 7.84 (d, J = 2.4 Hz, 1H), 7.78 (d, J = 2.5 Hz, 1H), 7.56 (d, J = 8.1 Hz, 1H), 7.44-7.35 (m, 1H), 7.21- 7.11 (m, 1H), 6.61 (s, 1H), 4.68-4.63 (m, 4H), 4.53- 4.44 (m, 1H), 4.27-4.04 (m, 4H), 3.50 (dd, J = 8.8, 6.6 Hz, 1H), 3.33-3.17 (m, 2H), 3.15-3.09 (m, 1H), 2.27- 2.17 (m, 1H), 2.02-1.87 (m, 1H). | MS (APCI+) m/z 623.3 (M + H)⁺ |
| Compound 74 | 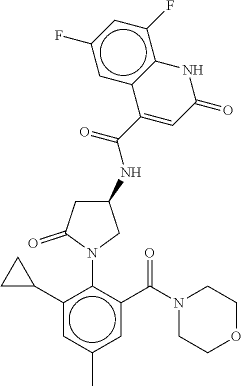 | ¹H NMR (400 MHz, DMSO- d₆) δ ppm 12.08 (s, 1H), 9.19 (dd, J = 146.8, 6.0 Hz, 1H), 7.67-7.58 (m, 1H), 7.48 (ddt, J = 18.2, 9.8, 2.0 Hz, 1H), 6.92 (d, J = 2.3 Hz, 1H), 6.89 (dd, J = 47.5, 1.9 Hz, 1H), 6.78 (d, J = 61.4 Hz, 1H), 4.70-4.59 (m, 1H), 4.10- 3.96 (m, 1H), 3.87-3.36 (m, 7H), 3.28-3.12 (m, 3H), 2.84 (ddd, J = 54.3, 16.9, 8.1 Hz, 1H), 2.56 (dd, J = 17.7, 4.5 Hz, 1H), 2.28 (s, 3H), 1.98-1.78 (m, 1H), 1.04- 0.74 (m, 3H), 0.62-0.50 (m, 1H). | (TFA+) rt: 0.78 (min; m/z: 551.2 |
| Compound 75 | 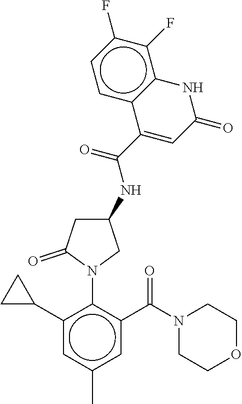 | ¹H NMR (400 MHz, DMSO- d₆, 90° C.) δ ppm 12.0-11.70 (m, 1H), 9.02 (d, J = 5.9 Hz, 0.5H), 8.71 (d, J = 6.2 Hz, 0.5H), 7.67 (dd, J = 16.0, 8.5 Hz, 1H), 7.26-7.15 (m, 1H), 6.93-6.79 (m, 2H), 6.73 (s, 0.5H), 6.57 (s, 0.5H + E79), 4.75-4.58 (m, 1H), 4.12- 3.97 (m, 1H), 3.77-2.41 (m, 11H), 2.29 (s, 3H), 1.99- 1.76 (m, 1H), 1.03-0.69 (m, 3H), 0.61-0.50 (m, 1H). | MS (ESI+) m/z 551.2 (M + H)⁺ |
| Compound 76 | 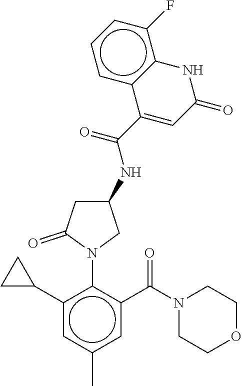 | ¹H NMR (600 MHz, DMSO- d₆) δ ppm 11.97 (d, J = 13.0 Hz, 1H), 9.16 (dd, J = 221.1, 6.2 Hz, 1H), 7.60 (dd, J = 18.0, 8.2 Hz, 1H), 7.51-7.38 (m, 1H), 7.20 (qd, J = 7.9, 4.9 Hz, 1H), 6.98-6.89 (m, 1H), 6.89-6.73 (m, 2H), 4.65 (s, 1H), 4.04 (dt, J = 33.4, 8.4 Hz, 1H), 3.69 (t, J = 14.5 Hz, 2H), 3.49 (q, J = 18.6, 17.1 Hz, 4H), 3.27-3.21 (m, 1H), 2.96-2.84 (m, 1H), 2.84- 2.70 (m, 1H), 2.29-2.28 (m, 3H), 1.97-1.75 (m, 2H), 0.95 (d, J = 45.2 Hz, 1H), 0.93-0.85 (m, 1H), 0.80 (dd, J = 23.2, 6.6 Hz, 2H), 0.63- 0.48 (m, 1H). | MS (ESI+) m/z 444 (M + H)⁺ |
| Compound 77 | 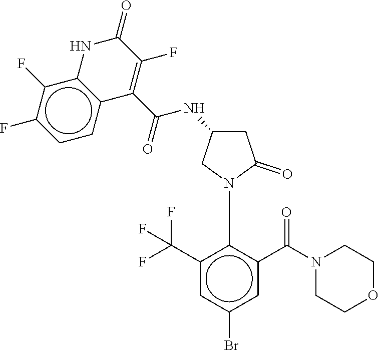 | ¹H NMR (500 MHz, DMSO- d₆) δ ppm 12.83 (s, 1H), 9.51 (d, J = 5.9 Hz, 0.5H), 9.34 (d, J = 6.3 Hz, 0.5H), 8.15-8.08 (m, 2H), 7.48-7.30 (m, 2H), 4.78-4.62 (m, 1H), 4.14 (t, J = 8.4 Hz, 0.5H), 3.99 (dd, J = 9.6, 7.3 Hz, 0.5H), 3.92-3.04 (m, 9H), 2.92 (dd, J = 17.0, 8.3 Hz, 0.5H), 2.83 (dd, J = 17.2, 8.5 Hz, 0.5H), 2.55- 2.42 (m, 1H). | MS (ESI+) m/z 661.2 (M + H)⁺ |
| Compound 78 | 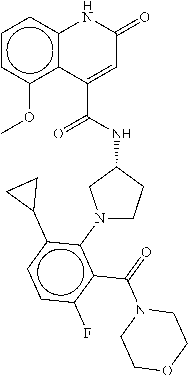 | ¹H NMR (400 MHz, DMSO- d₆) δ ppm 8.48-8.23 (dd, J = 46.9, 6.9 Hz, 1H), 7.52- 7.33 (t, J = 8.2 Hz, 1H), 6.98- 6.65 (m, 3H), 6.21-6.04 (d, J = 13.0 Hz, 1H), 4.49- 4.31 (m, 1H), 3.75-3.68 (s, 3H), 3.68-3.41 (m, 6H), 3.23-3.06 (m, 7H), 2.29- 2.12 (dddd, J = 15.4, 12.9, 7.6, 5.2 Hz, 1H), 2.11-1.98 (m, 1H), 1.94-1.77 (dddd, J = 26.6, 19.4, 11.0, 6.5 Hz, 1H), 1.00-0.74 (m, 2H), 0.74-0.47 (m, 2H). | MS (ESI+) m/z 535.3 (M + H)⁺ |
| Compound 79 | 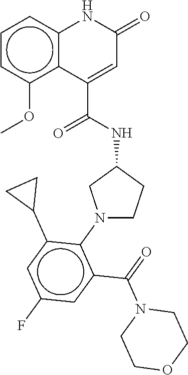 | ¹H NMR (500 MHz, DMSO- d₆) d 12.06 11.50 (s, 1H), 8.53 8.17 (dd, J = 62.6, 7.0 Hz, 1H), 7.62 7.36 (t, J = 8.2 Hz, 1H), 7.06 6.87 (d, J = 8.2 Hz, 1H), 6.87 6.59 (m, 3H), 6.33 6.05 (d, J = 18.4 Hz, 1H), 4.57 4.40 (m, 1H), 3.80 3.44 (m, 9H), 3.34 3.05 (m, 6H), 2.33 2.15 (dddd, J = 27.9, 15.5, 7.7, 3.5 Hz, 2H), 1.98 1.80 (m, 1H), 1.10 0.88 (m, 2H), 0.84 0.62 (m, 2H). | MS (ESI+) m/z 535.3 (M + H)⁺ |
| Compound 80 | 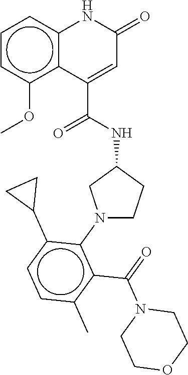 | ¹H NMR (500 MHz, DMSO- d₆) d 12.06 11.05 (s, 1H), 8.53 8.17 (dd, J = 62.6, 7.0 Hz, 1H), 7.62 7.36 (t, J = 8.2 Hz, 1H), 7.06 6.87 (d, J = 8.2 Hz, 1H), 6.87 6.59 (m, 3H), 6.33 6.05 (d, J = 18.4 Hz, 1H), 4.57 4.40 (m, 1H), 3.80 3.44 (m, 9H), 3.34 3.05 (m, 6H), 2.33 2.15 (dddd, J = 27.9, 15.5, 7.7, 3.5 Hz, 2H), 1.98 1.80 (m, 1H), 1.10 0.88 (m, 2H), 0.84 0.62 (m, 2H). | MS (ESI+) m/z 535.3 (M + H)⁺ |
| Compound 81 | 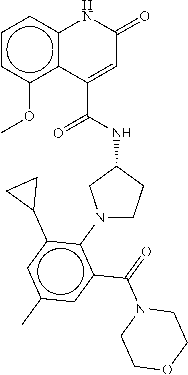 | ¹H NMR (500 MHz, DMSO- d₆) δ ppm 8.47-8.24 (dd, J = 58.1, 7.0 Hz, 1H), 7.56- 7.41 (t, J = 8.2 Hz, 1H), 7.02- 6.86 (dd, J = 8.3, 0.9 Hz, 1H), 6.82-6.60 (m, 2H), 6.26-6.08 (d, J = 17.4 Hz, 1H), 6.09-5.91 (s, 1H), 4.53- 4.38 (tt, J = 12.1, 6.5 Hz, 1H), 3.85-3.70 (d, J = 2.4 Hz, 3H), 3.70-3.39 (m, 10H), 3.18-3.09 (ddd, J = 14.0, 8.6, 5.4 Hz, 4H), 2.33- 2.09 (m, 4H), 2.02-1.78 (m, 1H), 1.06-0.87 (m, 2H), 0.82-0.55 (m, 2H). | MS (ESI+) m/z 531.3 (M + H)⁺ |
| Compound 82 | 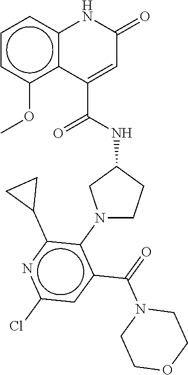 | ¹H NMR (500 MHz, DMSO- d₆) δ ppm 8.52-8.35 (m, 1H), 7.48-7.45 (t, J = 8.2 Hz, 1H), 7.10-7.08 (d, J = 5.9 Hz, 1H), 6.95-6.93 (d, J = 7.5 Hz, 1H), 6.76-6.74 (d, J = 8.3 Hz, 1H), 6.18-6.15 (d, J = 1 2.2 Hz, 1H), 4.52-4.39 (m, 1H), 3.74-3.73 (d, J = 3.9 Hz, 3H), 3.71-3.54 (m, 5H), 3.54-3.41 (m, 3H), 3.25- 3.05 (m, 5H), 2.41-2.36 (m, 1H), 2.31-2.23 (m, 1H), 1.95-1.84 (m, 1H), 1.03- 0.83 (m, 4H). | MS (ESI+) m/z 552.0 (M + H)⁺ |
| Compound 83 | 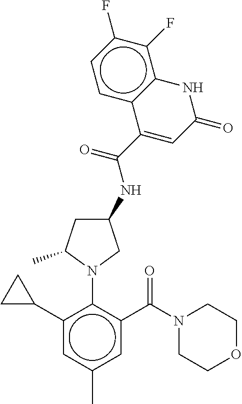 | ¹H NMR (600 MHz, DMSO- d₆) δ ppm 12.19 (s, 1H), 8.62 (d, J = 7.1 Hz, 1H), 7.72- 7.54 (m, 1H), 7.37-7.18 (m, 1H), 6.82-6.53 (m, 2H), 4.46 (dd, J = 7.8, 4.8 Hz, 1H), 3.68- 3.58 (m, 4H), 3.45-3.37 (m, 4H), 3.26 (dd, J = 10.9, 5.7 Hz, 1H), 3.06 (dd, J = 9.2, 2.7 Hz, 1H), 2.32-2.25 (m, 1H), 2.21 (s, 3H), 2.17-2.05 (m, 1H), 1.76 (ddd, J = 12.4, 8.7, 6.1 Hz, 1H), 1.02 (tdd, J = 9.2, 6.0, 3.9 Hz, 1H), 0.95 (d, J = 5.9 Hz, 3H), 0.88- 0.79 (m, 1H), 0.80-0.68 (m, 1H), 0.52 (dtd, J = 9.4, 5.8, 4.0 Hz, 1H). | MS (APCI+) m/z 551 (M + H)⁺ |
| Compound 84 | 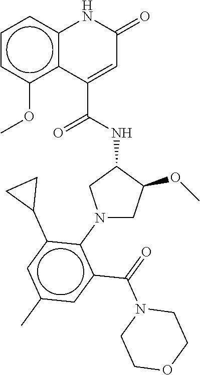 | ¹H NMR (500 MHz, DMSO- d₆) δ ppm 11.85 (s, 1H), 8.53- 8.17 (m, 1H), 7.47 (t, J = 8.2 Hz, 1H), 6.94 (dd, J = 8.3, 0.9 Hz, 1H), 6.76 (d, J = 8.2 Hz, 1H), 6.70 (d, J = 2.0 Hz, 1H), 6.67 (d, J = 4.8 Hz, 1H), 6.17 (d, J = 32.0 Hz, 1H), 4.32- 4.24 (m, 2H), 3.92 (d, J = 9.8 Hz, 1H), 3.75 (d, J = 8.6 Hz, 3H), 3.66-3.58 (m, 2H), 3.59- 3.40 (m, 5H), 3.36 (d, J = 6.1 Hz, 3H), 3.30-3.15 (m, 3H), 3.15-3.04 (m, 1H), 2.20 (s, 3H), 2.17-2.08 (m, 1H), 1.03-0.84 (m, 2H), 0.77- 0.67 (m, 1H), 0.67-0.57 (m, 1H). | MS (ESI+) m/z 561 (M + H)⁺. |
| Compound 85 | 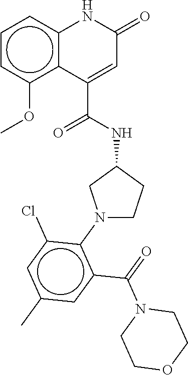 | ¹H NMR (600 MHz, DMSO- d₆) δ ppm 11.82 (s, 1H), 8.45- 8.26 (m, 1H), 7.46 (t, J = 8.2 Hz, 1H), 7.30 (ddd, J = 3.5, 2.1, 0.8 Hz, 1H), 6.96-6.88 (m, 2H), 6.79-6.70 (m, 1H), 6.51 (d, J = 0.6 Hz, 1H), 6.18 (dd, J = 16.7, 2.0 Hz, 1H), 4.44 (dh, J = 19.7, 7.0 Hz, 1H), 3.76 (d, J = 6.5 Hz, 3H), 3.63 (q, J = 4.0 Hz, 1H), 3.59 (d, J = 5.1 Hz, 1H), 3.57- 3.46 (m, 3H), 3.30-3.28 (m, 3H), 3.27-3.16 (m, 2H), 3.12 (dd, J = 8.7, 6.2 Hz, 1H), 2.29- 2.25 (m, 3H), 2.25-2.18 (m, 1H), 1.95-1.79 (m, 1H). | MS (ESI+) m/z 525 (M + H)⁺. |

TABLE 2
Biochemical Inhibition Data

| | SARS-CoV-2 | SARS | MERS | OC43 | 229E |
|---|---|---|---|---|---|
| | EC50 | EC50 | EC50 | EC50 | EC50 |
| Example | (μM) | (μM) | (μM) | (μM) | (μM) |
| Compound 1 | 0.00049 | 0.00197 | 0.00102 | 0.00076 | 0.00241 |
| Compound 2 | 0.00048 | 0.00224 | 0.00135 | 0.00309 | 0.00201 |
| Compound 3 | 0.00063 | 0.00097 | 0.00123 | 0.00388 | 0.0129 |
| Compound 4 | 0.00872 | 0.00993 | 0.0123 | 0.241 | 0.0398 |
| Compound 5 | 0.00048 | 0.0015 | 0.00126 | 0.00125 | 0.00144 |
| Compound 6 | 0.00078 | ND | 0.00251 | 0.00144 | 0.00232 |
| Compound 7 | 0.00043 | ND | 0.00099 | 0.00495 | 0.00276 |
| Compound 8 | 0.00043 | 0.00152 | 0.00148 | 0.00471 | 0.00334 |
| Compound 9 | 0.00047 | 0.00079 | 0.0018 | 0.00285 | 0.00201 |
| Compound 10 | 0.00054 | ND | 0.0015 | 0.012 | 0.00763 |
| Compound 11 | 0.00073 | ND | 0.00163 | 0.0103 | 0.00531 |
| Compound 12 | 0.00102 | ND | 0.00265 | 0.0234 | 0.0904 |
| Compound 13 | 0.00051 | ND | 0.00154 | 0.00256 | 0.00289 |
| Compound 14 | 0.00046 | 0.00273 | 0.00133 | 0.002 | 0.0021 |
| Compound 15 | 0.00077 | ND | 0.00328 | 0.00084 | ND |
| Compound 16 | 0.00061 | ND | 0.00146 | 0.00071 | ND |
| Compound 17 | 0.00048 | ND | 0.00127 | 0.0011 | ND |
| Compound 18 | 0.00044 | ND | 0.00135 | 0.00073 | ND |
| Compound 19 | 0.00044 | ND | 0.00144 | 0.00265 | ND |
| Compound 20 | 0.00054 | ND | 0.00168 | 0.00116 | ND |
| Compound 21 | 0.00055 | ND | 0.0013 | 0.0016 | ND |
| Compound 22 | 0.00077 | ND | 0.0015 | 0.0161 | ND |
| Compound 23 | 0.00051 | ND | 0.00126 | 0.00272 | ND |
| Compound 24 | 0.00052 | ND | 0.00156 | 0.00074 | ND |
| Compound 25 | 0.00044 | ND | 0.00141 | 0.00212 | ND |
| Compound 26 | 0.00045 | ND | 0.00128 | 0.00035 | ND |
| Compound 27 | 0.00063 | ND | 0.00136 | 0.00872 | ND |
| Compound 28 | 0.00042 | 0.0014 | 0.00122 | 0.00154 | 0.00304 |
| Compound 29 | 0.0139 | ND | 0.00885 | 0.378 | 0.682 |
| Compound 30 | 0.00042 | ND | 0.00104 | 0.00058 | 0.00125 |
| Compound 31 | 0.0005 | ND | 0.00194 | 0.00177 | 0.00281 |
| Compound 32 | 0.00045 | ND | 0.00106 | 0.00117 | 0.00178 |
| Compound 33 | 0.00092 | ND | 0.00249 | 0.171 | 0.0385 |
| Compound 34 | 0.00112 | 0.00298 | 0.00228 | 0.0918 | 0.0417 |
| Compound 35 | 0.00053 | 0.00245 | 0.00093 | 0.0165 | 0.0026 |
| Compound 36 | 0.00049 | 0.00143 | 0.00102 | 0.00084 | 0.00239 |
| Compound 37 | 0.00063 | 0.00168 | 0.00122 | 0.001 | 0.00253 |
| Compound 38 | 0.00184 | ND | 0.00239 | 0.00911 | 0.0059 |
| Compound 39 | 0.00042 | 0.00256 | 0.00112 | 0.00105 | 0.00345 |
| Compound 40 | 0.00042 | ND | 0.00089 | 0.00055 | 0.00247 |
| Compound 41 | 0.00045 | ND | 0.00082 | 0.00069 | 0.00216 |
| Compound 42 | 0.00045 | ND | 0.00105 | 0.0014 | 0.00358 |
| Compound 43 | 0.00046 | ND | 0.00141 | 0.00317 | 0.015 |
| Compound 44 | 0.00053 | ND | 0.00107 | 0.00198 | 0.00318 |
| Compound 45 | 0.00042 | ND | 0.0007 | 0.00051 | 0.00152 |
| Compound 46 | 0.00047 | ND | 0.00175 | 0.00081 | 0.00276 |
| Compound 47 | 0.0099 | ND | 0.00961 | 0.138 | 0.117 |
| Compound 48 | 0.00043 | 0.00115 | 0.0009 | 0.00044 | 0.00143 |
| Compound 49 | 0.00043 | 0.0011 | 0.00095 | 0.00164 | 0.0047 |
| Compound 50 | 0.0005 | 0.00223 | 0.0019 | 0.0012 | 0.00451 |
| Compound 51 | 0.00042 | 0.00123 | 0.00068 | 0.0011 | 0.00356 |
| Compound 52 | 0.00043 | ND | 0.00103 | 0.00084 | 0.00358 |
| Compound 53 | 0.0006 | ND | 0.00186 | 0.00071 | 0.00198 |
| Compound 54 | 0.00043 | ND | 0.00112 | 0.00042 | 0.00472 |
| Compound 55 | 0.00042 | 0.00056 | 0.00084 | 0.00042 | 0.00174 |
| Compound 56 | 0.00045 | 0.00449 | 0.00092 | 0.00074 | 0.00183 |
| Compound 57 | 0.00042 | ND | 0.00053 | 0.00042 | 0.00088 |
| Compound 58 | 0.00042 | 0.00107 | 0.00091 | 0.00584 | 0.0245 |
| Compound 59 | 0.00111 | ND | 0.00101 | 0.00609 | 0.0204 |
| Compound 60 | 0.00048 | 0.00074 | 0.0014 | 0.00122 | 0.00514 |
| Compound 61 | 0.00193 | 0.00461 | 0.00325 | 0.0523 | 0.367 |
| Compound 62 | 0.0008 | ND | 0.00194 | 0.00463 | 0.0684 |
| Compound 63 | 0.00056 | 0.00127 | 0.00106 | 0.00091 | 0.00199 |
| Compound 64 | 0.00053 | ND | 0.00138 | 0.00287 | 0.00961 |
| Compound 65 | 0.00042 | 0.00186 | 0.00154 | 0.00129 | 0.00276 |
| Compound 66 | 0.00048 | ND | 0.00128 | 0.00206 | 0.00578 |
| Compound 67 | 0.0005 | 0.00256 | 0.0015 | 0.00152 | 0.00422 |
| Compound 68 | 0.0006 | ND | 0.00183 | 0.00355 | 0.0104 |
| Compound 69 | 0.00067 | ND | 0.00176 | 0.00492 | 0.0155 |
| Compound 70 | 0.00102 | ND | 0.00196 | 0.0111 | 0.0337 |
| Compound 71 | 0.00042 | 0.00194 | 0.00078 | 0.00066 | 0.00384 |
| Compound 72 | 0.00111 | ND | 0.00283 | 0.0192 | 0.039 |
| Compound 73 | 0.00053 | 0.00384 | 0.00169 | 0.00113 | 0.00579 |
| Compound 74 | 0.00059 | 0.00143 | 0.00129 | 0.0034 | 0.00724 |
| Compound 75 | 0.00063 | 0.00072 | 0.00172 | 0.00225 | 0.00611 |
| Compound 76 | 0.00054 | ND | 0.00216 | 0.00146 | 0.00448 |
| Compound 77 | 0.00121 | ND | 0.00169 | 0.0283 | 0.124 |
| Compound 78 | 0.00142 | ND | 0.00231 | 0.00145 | 0.00199 |
| Compound 79 | 0.00042 | ND | 0.00151 | 0.00076 | 0.00242 |
| Compound 80 | 0.00337 | 0.00274 | 0.00412 | 0.0012 | 0.00231 |
| Compound 81 | 0.00049 | 0.00138 | 0.00231 | 0.00066 | 0.00213 |
| Compound 82 | 0.00099 | ND | 0.00291 | 0.00161 | 0.0135 |
| Compound 83 | 0.00042 | 0.00153 | 0.00139 | 0.00042 | 0.00176 |
| Compound 84 | 0.0006 | ND | 0.00244 | 0.00064 | 0.00235 |
| Compound 85 | 0.0014 | 0.00313 | 0.00154 | 0.00227 | 0.00916 |

ND = Not Determined

TABLE 3
Antiviral Data

| | Antiviral CoV-2 | Antiviral 229E | Antiviral OC43 |
|---|---|---|---|
| Example | EC50 (μM) | EC50 (μM) | EC50 (μM) |
| Compound 1 | 0.008 | 0.042 | 0.041 |
| Compound 2 | 0.0115 | 0.066 | 0.24 |
| Compound 3 | 0.0203 | ND | ND |
| Compound 4 | ND | ND | ND |
| Compound 5 | 0.0102 | 0.039 | 0.047 |
| Compound 6 | 0.0219 | ND | ND |
| Compound 7 | 0.0256 | ND | ND |
| Compound 8 | 0.019 | 0.25 | 0.88 |
| Compound 9 | 0.3189 | 0.11 | 0.28 |
| Compound 10 | 0.5886 | ND | ND |
| Compound 11 | ND | ND | ND |
| Compound 12 | ND | ND | ND |
| Compound 13 | 0.0196 | ND | ND |
| Compound 14 | 0.0129 | 0.15 | 0.19 |
| Compound 15 | ND | ND | ND |
| Compound 16 | ND | ND | ND |
| Compound 17 | ND | ND | ND |
| Compound 18 | 0.0136 | ND | ND |
| Compound 19 | ND | ND | ND |
| Compound 20 | ND | ND | ND |
| Compound 21 | ND | ND | ND |
| Compound 22 | ND | ND | ND |
| Compound 23 | 0.0319 | ND | ND |
| Compound 24 | ND | ND | ND |
| Compound 25 | ND | ND | ND |
| Compound 26 | ND | ND | ND |
| Compound 27 | 2.805 | ND | ND |
| Compound 28 | 0.077 | 0.8949 | 0.1526 |
| Compound 29 | ND | ND | ND |
| Compound 30 | ND | 0.011 | 0.029 |
| Compound 31 | 0.0088 | 0.073 | 0.141 |
| Compound 32 | 0.0186 | ND | ND |
| Compound 33 | 0.174 | ND | ND |
| Compound 34 | ND | 3.366 | 7.319 |
| Compound 35 | ND | ND | ND |
| Compound 36 | 0.0091 | 0.0578 | 0.04867 |
| Compound 37 | 0.0288 | ND | ND |
| Compound 38 | ND | 0.1422 | 0.3268 |
| Compound 39 | 0.0189 | ND | ND |
| Compound 40 | 0.005 | ND | ND |
| Compound 41 | ND | ND | ND |
| Compound 42 | 0.0082 | ND | ND |
| Compound 43 | 0.0247 | ND | ND |
| Compound 44 | ND | ND | ND |
| Compound 45 | 0.0186 | 0.05605 | <0.03 |
| Compound 46 | 0.0209 | 0.073 | 0.049 |
| Compound 47 | ND | ND | ND |
| Compound 48 | 0.0058 | 0.044 | 0.031 |
| Compound 49 | 0.0113 | 0.09587 | 0.168 |
| Compound 50 | 0.0114 | 0.3193 | 0.3035 |
| Compound 51 | 0.016 | 0.04746 | 0.05837 |
| Compound 52 | 0.009 | ND | ND |
| Compound 53 | ND | ND | ND |
| Compound 54 | 0.005 | ND | ND |
| Compound 55 | 0.0046 | ND | ND |
| Compound 56 | 0.0181 | 0.062 | 0.014 |
| Compound 57 | ND | 0.08303 | 0.095 |
| Compound 58 | ND | 0.9834 | 0.8643 |
| Compound 59 | 0.0956 | ND | ND |
| Compound 60 | 0.1805 | ND | ND |
| Compound 61 | 0.4129 | 63.07 | 11.44 |
| Compound 62 | 0.0218 | ND | ND |
| Compound 63 | 0.0127 | ND | ND |
| Compound 64 | ND | ND | ND |
| Compound 65 | 0.0092 | ND | ND |
| Compound 66 | ND | ND | ND |
| Compound 67 | ND | ND | ND |
| Compound 68 | ND | ND | ND |
| Compound 69 | ND | ND | ND |
| Compound 70 | ND | ND | ND |
| Compound 71 | ND | ND | ND |
| Compound 72 | ND | ND | ND |
| Compound 73 | 0.0123 | ND | ND |
| Compound 74 | 0.052 | 0.248 | 0.4288 |
| Compound 75 | 0.0231 | ND | ND |
| Compound 76 | ND | ND | ND |
| Compound 77 | ND | ND | ND |
| Compound 78 | 0.4602 | 0.04807 | 0.0978 |
| Compound 79 | 0.0385 | 0.02842 | 0.02 |
| Compound 80 | 0.5551 | ND | ND |
| Compound 81 | 0.0182 | 0.073 | 0.013 |
| Compound 82 | 0.2411 | 1.17 | 0.083 |
| Compound 83 | ND | 0.029 | 0.023 |
| Compound 84 | 0.0351 | 0.141 | 0.019 |
| Compound 85 | 0.2739 | 0.3824 | 0.21 |

ND = Not Determined

Antiviral Activity of Compound 2 Against SARS-CoV-2 and Other Human and Animal Coronaviruses

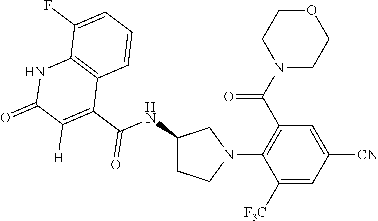

Compound 2 demonstrated high in vitro potency in a SARS-CoV-2 Mpro biochemical assays and in SARS CoV-2 antiviral assays using HeLa cells expressing ACE2 (HeLa-ACE2) or primary HBEC (Table 4).

TABLE 4
In Vitro Potency of Compound 2

| | Average (SD) | |
|---|---|---|
| Biochemical | | |
| SARS-CoV-2 Ki (nM) | 0.15 | (0.003) |
| Antiviral* | | |
| SARS-CoV-2 HeLa-ACE2 EC₅₀ (nM) | 11.3 | (1.5) |
| SARS-CoV-2 HeLa-ACE2 EC₉₀ (nM) | 25 | (4.9) |
| SARS-CoV-2 HBEC EC₅₀ (nM) | 6.7 | (3.5) |
| SARS-CoV-2 HBEC EC₉₀ (nM) | 12 | (6.2) |

*Strain USA-WA1/2020.

In the above antiviral assays with HeLa-ACE2, Compound 2 exhibited a CC₅₀>40,000 nM producing a selectivity index (CC₅₀/EC₅₀) that exceeded 3,500. The cytotoxicity of Compound 2 was also evaluated with other cell lines: it had CC 50 values >100,000 nM in human cell lines such as A549 and Huh-7, and in an African green monkey cell line, Vero. Mitochondrial toxicity potential of Compound 2 was assessed in multiple mitochondrial rich cell types (proliferating and non-proliferating) in glucose- and galactose-based media. The study included 3 proliferative cell types (A172, PC3, and HepG2) as well as IPSC-derived cardiomyocytes (non-proliferating) treated with Compound 2 with and without valspodar, a P-gp inhibitor, in both types of media. The positive control, rotenone, showed a pronounced increase in cytotoxicity in galactose-based media (restricting energy production to mitochondrial oxidative phosphorylation) for all 4 cell types, clearly indicating mitochondrial toxicity. By contrast, Compound 2 showed no toxicity in either medium at concentrations up to 300 μM (the highest dose tested), indicating a lack of mitochondrial toxicity.

The Mpro catalytic site is highly conserved across human coronaviruses, and, therefore, the potency of Compound 2 was evaluated in biochemical Mpro assays for a panel of 14 coronaviruses known to infect humans or animals (including 2 bat coronaviruses with high risk for spillover to humans) (Table 5). These 14 coronaviruses encompass members from each of the 4 genera of coronaviruses including alpha, beta, gamma, and delta: SARS-CoV-2, SARS-CoV, MERS-CoV, OC43, 229E, HKU1, NL63, MHV, WIV1, HKU9, FIPV, PEDV, IBV, and HKU11. Compound 2 exhibited a Ki of 0.15 nM for SARS-CoV-2 Mpro in a biochemical assay. In addition, Compound 2 retained high potency (≤12-fold change relative to SARS-CoV-2 Ki) for Mpro from 4 additional human coronaviruses (SARS-CoV, MERS-CoV, OC43, and 229E), while showing weaker potency against Mpro from NL63 and HKU1. Compound 2 also demonstrated high biochemical potency for Mpro from animal coronaviruses such as WIV1, FIPV, and PEDV.

TABLE 5
In Vitro Activity of Compound 2 for Mpro from
Human or Animal Coronaviruses Relative to Activity
Against SARS-CoV-2 in Biochemical Assays

| | Fold change,^{a} Average (SD) | |
|---|---|---|
| Human Coronavirus | | |
| SARS-CoV | 2.5 | (1.0) |
| MERS-CoV | 3.0 | (0.5) |
| OC43 | 11.9 | (3.1) |
| 229E | 5.7 | (2.2) |
| NL63 | 31 | (11) |
| HKU1 | 22,178 | (8,122) |
| Animal Coronavirus | | |
| MHV (mouse) | 67 | (26) |
| WIV1 (bat) | 2.4 | (0.7) |
| HKU9 (bat) | 78 | (35) |
| FIPV (feline) | 4.9 | (2.7) |
| PEDV (porcine) | 4.4 | (1.8) |
| IBV (avian) | 83 | (32) |
| HKU11 (avian) | 477 | (162) |

^{a}Ki fold change relative to Compound 2 Ki for SARS-CoV-2 (Wuhan-Hu-1 strain) Mpro (0.15 nM).

When tested against a panel of human proteases (including thrombin, elastase, caspase-2, cathepsin B, cathepsin D, cathepsin F, cathepsin K, cathepsin L, cathepsin S, cathepsin V [cathepsin L2], and chymotrypsin), pig protease pepsin, and HIV-1 viral protease, Compound 2 demonstrated no apparent inhibition for any of these proteases at 10 μM (20 μM for cathepsin F), indicating Compound 2 has high specificity for SARS-CoV-2 Mpro.

The Mpro catalytic site is highly conserved across human coronaviruses, and, therefore, the potency of compounds was evaluated in biochemical Mpro assays for a panel of 14 coronaviruses known to infect humans or animals (including 2 bat coronaviruses with high risk for spillover to humans) (Table 6). These 14 coronaviruses encompass members from each of the 4 genera of coronaviruses including alpha, beta, gamma, and delta: SARS-CoV-2, SARS-CoV, MERS-CoV, OC43, 229E, HKU1, NL63, MHV, WIV1, HKU9, FIPV, PEDV, IBV, and HKU11. Compounds 1 and 2 retain high potency (≤12-fold change relative to SARS-CoV-2 Ki) for Mpro from 4 additional human coronaviruses (SARS-CoV, MERS-CoV, OC43, and 229E), while showing weaker potency against Mpro from NL63 and HKU1. Compounds 1 and 2 also demonstrate high biochemical potency for Mpro from animal coronaviruses such as WIV1, FIPV, and PEDV.

TABLE 6
In Vitro Ki Fold Change for Human or Animal Coronaviruses Relative
to Activity Against SARS-CoV-2 in Biochemical Assays^{a}

| | Com- | Com- | | |
|---|---|---|---|---|
| | pound 1 | pound 2 | nirmatrelvir | ensitrelvir |
| Human | | | | |
| Coronavirus | | | | |
| SARS-CoV-2 Ki (nM) | 0.16 | 0.15 | 0.20 | 0.51 |
| SARS-CoV | 3.4 | 2.5 | 2 | 3 |
| MERS-CoV | 2.6 | 3.0 | 2.1 | 9 |
| OC43 | 2.5 | 11.9 | 1.9 | 5.3 |
| 229E | 7.2 | 5.7 | 31 | 459 |
| NL63 | 60.6 | 31 | 52 | 1700 |
| HKU1 | 8,710 | 22,178 | 65 | 852 |
| Animal | | | | |
| Coronavirus | | | | |
| MHV (mouse) | 29 | 67 | 3.6 | 47 |
| WIV1 (bat) | 4.2 | 2.4 | 1 | 2.2 |
| HKU9 (bat) | 38 | 78 | 28 | 43 |
| FIPV (feline) | 3.7 | 4.9 | 2 | 382 |
| PEDV (porcine) | 3.4 | 4.4 | 4.9 | 33 |
| IBV (avian) | 34 | 83 | 20 | 2,000 |
| HKU11 (avian) | 2,064 | 477 | 50 | 9,945 |

^{a}Ki fold change relative to Ki for SARS-CoV-2 (Wuhan-Hu-1 strain) Mpro.

Compounds 1 and 2 demonstrated high in vitro potency in a SARS-CoV-2 Mpro biochemical assay and in SARS-CoV-2 antiviral assays using HeLa cells expressing ACE2 (HeLa-ACE2) or primary human bronchial epithelial cells (HBEC) (Table 7).

TABLE 7
In Vitro SARS-CoV-2 Antiviral Potency in ACE2 (HeLa-ACE2)
or primary human bronchial epithelial cells (HBEC)

| Assay | Compound 1 | | Compound 2 | |
|---|---|---|---|---|
| SARS-CoV-2 HeLa-ACE2 EC₉₀ (nM) | 11.3 | (1.5) | 8.1 | (1.7) |
| SARS-CoV-2 HeLa-ACE2 EC₉₀ (nM) | 25 | (4.9) | 18.1 | (4.5) |
| SARS-CoV-2 HBEC EC₅₀ (nM) | 6.7 | (3.5) | 4.7 | (1.2) |
| SARS-CoV-2 HBEC EC₉₀ (nM) | 12 | (6.2) | 8.0 | (1.7) |

* Strain USA-WA1/2020.

### ALIHBEC Culture Conditions

Normal primary human bronchial epithelial cells (HBECs) (Lonza, Part No. CC-2540, Lot 18TL057583) are cultured at 37° C. 50% CO₂ in Millicell-96 cell culture insert plates with 1 μm PET filters (MilliporeSigma, Part No. PSRPOO4R5) at an air liquid interface (ALI) for at least 4 weeks using PneumaCult-ALI Medium (STEMCELL Technologies, Part No. 05001). HBECs are first expanded in cell culture flasks before seeding 10,000 cells per well submerged in PneumaCult Ex-Plus Medium (STEMCELL Technologies, Part No. 05040). After 1 week, cells are switched into PneumaCult-ALI Medium and ALI is initiated by removing medium from the apical surface. The medium is exchanged every 2-3 days for at least 4 weeks and the air-liquid interface is maintained to allow for differentiation of the cells. One day prior to infection, the cells are transferred to 96-well receiver plates (MilliporeSigma, Part No. MACACORSS) and transported to the BSL-3 facility to equilibrate.

SARS-CoV-2/ALI HBEC Screening Assay

On the day of infection, compounds are manually diluted in PneumaCult-ALI media in deep 96-well plates. Shortly before infection, the apical surface of the cells is washed once with DPBS with calcium and magnesium that has been pre-warmed in a 37° C. water bath (ThermoFisher Scientific, 14040133), and the basolateral media is exchanged for the compound dilutions in media. 41,450 PFU SARS-CoV-2 strain USA/WA1/2020 is added to the apical surface in 50 μL DPBS and allowed to incubate for 2 h at 34° C. 5% CO₂, after which the inoculum is removed, and cells are washed once with DPBS. Compound and media are refreshed at 24 and 48 h post-infection. Apical washes are collected at 72 h post-infection by adding 150 μL DPBS to the apical surface for 15 minutes. Apical washes are stored at −80° C. until RNA isolation using the PureLink Pro 96 Viral RNA/DNA Purification Kit (ThermoFisher, Part No. 12280096A) and analyzed for viral RNA levels by RT-qPCR using the SuperScript III Platinum One-Step qRT-PCR Kit (ThermoFisher, Part No. 11732088) and the 2019-nCoV N1 CDC Primers and Probe Set (Integrated DNA Technologies, Part Nos. 10006830, 10006831, 10006832).

### Cytotoxicity Screening

Cytotoxicity is assessed at 72 h post infection using a Cytotoxicity Detection kit (LDH) (Sigma, Part No. 11644793001). 100 μL of media is collected from the basolateral chambers and transferred to a 96-well flat-bottom plate (Corning, Part No. 3599). The reaction mixture is prepared fresh for each assay, and 100 μL is added to the well. Plates are incubated at room temperature for 30 minutes, protected from light, before absorbance is measured at 490 nm using a CLARIOstar Plus microplate reader (BMG Labtech).

### Data Analysis

Viral load (pfu equivalents/mL) for each sample is determined based on a standard curve generated using RNA isolated from serial dilutions of the stock virus. Viral load log 10 reductions are then determined for each experimental compound compared to the neutral DMSO control. For dose-response experiments, data are uploaded to Genedata Screener, Version 16.0; PFU equivalents/mL are normalized to neutral (DMSO) minus inhibitor controls (5 μM nafamostat) and curves are fitted with the four parameter Hill Equation. For cytotoxicity screening, averages are taken for the experimental samples and presented as a percentage of the positive control puromycin (5 μM). Technical triplicates are run for both antiviral and cytotoxicity readouts.

TABLE 8
Parallel artificial membrane permeability assay
(PAMPA) and Unbound Microsomal Clearance Data

| | | Clint, mic | Clint, mic |
|---|---|---|---|
| | PAMPA | Unbound | Unbound |
| Example | (10E−6 cm/s) | (L/hr/kg) (Rat) | (L/hr/kg) (Human) |
| Compound 1 | 16.2 | 19.8 | 5.8 |
| Compound 2 | 6.42 | <3.6 | <2.1 |
| Compound 3 | 5.84 | <3.6 | <2.2 |
| Compound 4 | 5.55 | <2.5 | <1.5 |
| Compound 5 | 7.23 | <3.6 | <2.2 |
| Compound 6 | 3.22 | 4.9 | <2.1 |
| Compound 7 | 6.77 | <3.5 | <2.1 |
| Compound 8 | 5.11 | <3.6 | <2.1 |
| Compound 9 | 0.34 | <3.5 | <2.1 |
| Compound 10 | 0.70 | <3.0 | <1.8 |
| Compound 11 | 0.49 | <3.7 | |
| Compound 12 | 0.23 | <2.5 | <1.5 |
| Compound 13 | 3.48 | <2.5 | <1.5 |
| Compound 14 | 2.73 | 3.4 | <1.8 |
| Compound 15 | 15.2 | 13.2 | 4.6 |
| Compound 16 | 7.38 | 18.8 | 5.6 |
| Compound 17 | 4.60 | <2.9 | <1.7 |
| Compound 18 | 7.19 | 5.7 | 2.3 |
| Compound 19 | 6.68 | 12.0 | <2.2 |
| Compound 20 | 13.8 | 8.5 | <2.2 |
| Compound 21 | 8.89 | 7.7 | 3.3 |
| Compound 22 | 7.51 | <3.7 | <2.2 |
| Compound 23 | 4.12 | 4.1 | <2.2 |
| Compound 24 | 2.68 | 14.7 | 3.6 |
| Compound 25 | 2.06 | | |
| Compound 26 | 4.94 | <3.4 | 4.1 |
| Compound 27 | 0.11 | <3.5 | <2.1 |
| Compound 28 | 1.23 | 3.9 | <2.2 |
| Compound 29 | 3.51 | <3.1 | <1.8 |
| Compound 30 | 13.3 | 18.2 | 3.7 |
| Compound 31 | 11.8 | 27.4 | 5.5 |
| Compound 32 | 7.29 | <4.2 | 2.9 |
| Compound 33 | 1.12 | <3.4 | <2.1 |
| Compound 34 | 1.04 | <2.7 | <1.6 |
| Compound 35 | 0.10 | 2.8 | <1.7 |
| Compound 36 | 14.3 | 24.2 | 5.7 |
| Compound 37 | 6.23 | 25.4 | 9.0 |
| Compound 38 | 14.5 | 37.5 | 10.4 |
| Compound 39 | 4.71 | <2.5 | <1.5 |
| Compound 40 | 5.15 | 13.2 | <1.7 |
| Compound 41 | 0.59 | 16.8 | 8.8 |
| Compound 42 | 14.7 | 28.9 | 7.5 |
| Compound 43 | 12.7 | 18.7 | 6.0 |
| Compound 44 | 15.3 | 17.5 | 2.5 |
| Compound 45 | 14.7 | 56.5 | 21.1 |
| Compound 46 | 15.2 | 53.8 | 3.2 |
| Compound 47 | 15.7 | 10.9 | 2.8 |
| Compound 48 | 17.9 | 12.6 | 4.9 |
| Compound 49 | 15.6 | 34.4 | 11.4 |
| Compound 50 | 16.2 | 9.9 | 11.4 |
| Compound 51 | 14.5 | 26.8 | 7.5 |
| Compound 52 | 12.6 | 27.1 | 11.0 |
| Compound 53 | 8.90 | 36.9 | 13.0 |
| Compound 54 | 13.1 | 15.4 | 8.4 |
| Compound 55 | 18.6 | 19.1 | 9.4 |
| Compound 56 | 10.9 | 24.0 | 9.4 |
| Compound 57 | 4.09 | 26.2 | 7.5 |
| Compound 58 | 17.9 | 60.1 | 21.9 |
| Compound 59 | 8.24 | <4.1 | <3.7 |
| Compound 60 | 2.95 | | 4.0 |
| Compound 61 | 15.7 | 5.0 | 3.1 |
| Compound 62 | 4.73 | <2.5 | <1.5 |
| Compound 63 | 5.60 | 43.3 | 21.4 |
| Compound 64 | 14.9 | 64.0 | 16.3 |
| Compound 65 | 16.0 | 15.5 | 5.6 |
| Compound 66 | 15.0 | 36.4 | 17.8 |
| Compound 67 | 16.3 | 70.5 | 18.8 |
| Compound 68 | 14.7 | 67.1 | 19.0 |
| Compound 69 | 14.5 | 62.5 | 18.2 |
| Compound 70 | 13.9 | 50.7 | 12.3 |
| Compound 71 | 18.8 | 21.5 | 25.4 |
| Compound 72 | 19.6 | 76.9 | 33.7 |
| Compound 73 | 11.7 | 19.6 | 7.0 |
| Compound 74 | 3.54 | <3.4 | <2.0 |
| Compound 75 | 2.46 | <3.3 | <2.0 |
| Compound 76 | 2.44 | <4.4 | <2.6 |
| Compound 77 | 3.46 | <3.9 | <2.3 |
| Compound 78 | 2.46 | 25.1 | 11.3 |
| Compound 79 | 2.98 | 14.1 | 1.9 |
| Compound 80 | 2.30 | 25.1 | 9.6 |
| Compound 81 | 2.81 | 13.6 | 3.9 |
| Compound 82 | 3.10 | 3.1 | 3.4 |
| Compound 83 | 15.7 | 85.7 | 66.9 |
| Compound 84 | 3.60 | 11.7 | 7.2 |
| Compound 85 | 3.19 | 17.3 | 6.2 |

Microsomal Stability: Analogs (0.5 μM) were incubated with rat or human microsomes (0.25 mg/mL) at pH 7.4 (KPO₄ buffer), 37° C. for 0, 5, 10, 15, 20 and 30 minutes in the presence of NADPH. The experiments were stopped with 1:1 CH₃CN:MeOH and analyzed by LC-MS/MS. An internal standard of carbutamide was used. Table 8.

In a panel of off-target binding assays, Compound 2 (10 μM) had no displacement of control-specific binding or enzyme inhibition by >50%. When tested against a panel of human proteases (including thrombin, elastase, caspase-2, cathepsin B, cathepsin D, cathepsin F, cathepsin K, cathepsin L, cathepsin S, cathepsin V [cathepsin L2], and chymotrypsin), pig protease pepsin, and HIV-1 viral protease, Compound 2 demonstrated no apparent inhibition for any of these proteases at 10 μM (20 μM for cathepsin F).

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof. All publications, patents, and patent applications cited herein are hereby incorporated by reference in their entirety for all purposes.

What is claimed:

1. A compound of Formula (III), or a pharmaceutically acceptable salt thereof, 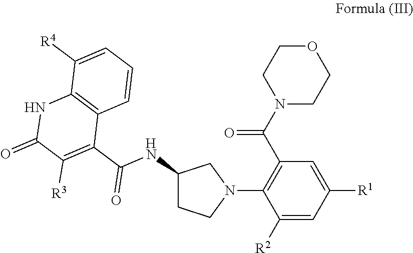
wherein:
R¹ is CN or Br;
R² is CF₃ or cyclopropyl;
R³ is H or F; and
R⁴ is H or F.

2. The compound of claim 1, or a pharmaceutically acceptable salt thereof, wherein the compound has the structure

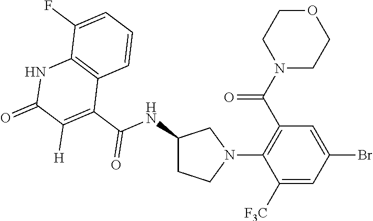

3. The compound of claim 1, or a pharmaceutically acceptable salt thereof, wherein the compound has the structure

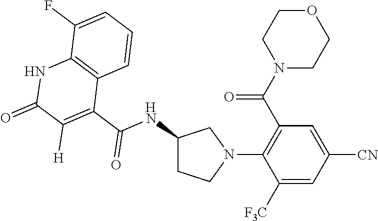

4. A compound of Formula (VI), or a pharmaceutically acceptable salt thereof, 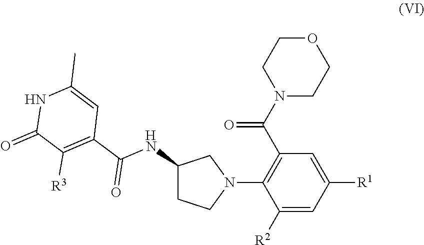
wherein:
R¹ is CN or Br;
R² is CF₃ or cyclopropyl; and
R³ is H or F.

5. The compound of claim 4, or a pharmaceutically acceptable salt thereof, wherein the compound has the structure

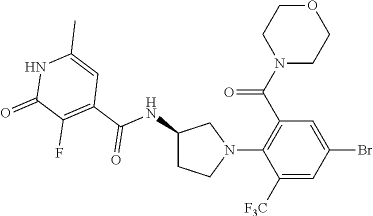

6. A pharmaceutical composition comprising a compound as described in claim 1 and a pharmaceutically acceptable excipient.

7. A method of treating a subject suffering from a COVID-19 infection comprising administering a effective amount of a compound as described in claim 1 to the subject to treat the COVID-19 infection.

8. A compound represented by the following structure [chemical structure]
